(19) United States
(12) Patent Application Publication
(10) Pub. No.: US 2024/0199757 A1
(43) Pub. Date: Jun. 20, 2024

(54) CD73 (NT5E) TARGETING POLYPEPTIDES

(71) Applicant: THE UNIVERSITY OF CHICAGO, Chicago, IL (US)

(72) Inventors: Anthony KOSSIAKOFF, Chicago, IL (US); Carla E. MEINTS, Chicago, IL (US); Somnath MUKHERJEE, Chicago, IL (US)

(73) Assignee: THE UNIVERSITY OF CHICAGO, Chicago, IL (US)

(21) Appl. No.: 18/590,875

(22) Filed: Feb. 28, 2024

Related U.S. Application Data
(63) Continuation of application No. PCT/US2023/069531, filed on Jun. 30, 2023.
(60) Provisional application No. 63/357,477, filed on Jun. 30, 2022.

(51) Int. Cl. C07K 16/28; A61K 39/00; A61P 35/00; G01N 33/574

(52) U.S. Cl. CPC C07K 16/2896; A61P 35/00; G01N 33/57484; A61K 2039/505; C07K 2317/21; C07K 2317/24; C07K 2317/622; C07K 2317/76; C07K 2317/92

(57) ABSTRACT

Aspects of the present disclosure are directed to NT5E (CD73)-targeting polypeptides, including antibodies, antibody-drug conjugates, antibody fragments, antibody-like molecules, and chimeric receptors. Also disclosed herein are nucleic acids encoding for such NT5E-targeting polypeptides and cells comprising such nucleic acids. Described are methods for treatment of cancer using NT5E-targeting polypeptides.

### CROSS-REFERENCE TO RELATED APPLICATIONS

This application is a continuation of International Application No. PCT/US2023/069531, filed Jun. 30, 2023, which claims the benefit of priority to U.S. Provisional Patent Application Ser. No. 63/357,477, filed Jun. 30, 2022, each of which are hereby incorporated by reference in their entirety.

This invention was made with government support under grant number CA258297 awarded by the National Institutes of Health. The government has certain rights in the invention.

### SEQUENCE LISTING

The instant application contains a Sequence Listing which has been submitted in XML format and is hereby incorporated by reference in its entirety. Said XML copy, created on Feb. 27, 2024, is named “ARCDP0761US1_ST26.XML” and is 66,238 bytes in size.

### BACKGROUND

### I. Field of the Invention

Aspects of the invention relate to at least the fields of molecular biology and medicine.

### II. Background

NT5E (also known as “CD73”) is a 70 kDa 5′-ectonucleotidase that is tethered by a glycosylphosphatidyl anchor to the extracellular side of many cell types including endothelial, T lymphocyte, macrophage, and dendritic. NT5E, together with NTPDase-1 CD39, is an important regulator of the inflammatory response. Cell death or stress triggers release of ATP which activates pro-inflammatory and immunostimulatory pathways. ATP is down-regulated by CD39-mediated sequential hydrolysis to ADP and AMP. NT5E dephosphorylates AMP to adenosine which then stimulates anti-inflammatory and immunosuppressive responses.

Many cancerous tissues highly overexpress NT5E, including triple negative breast cancer, pancreatic cancer, glioma, melanoma, prostate cancer, bladder cancer, leukemia, esophageal cancer, and ovarian cancer. Increased cell surface NT5E generates an elevated, local concentration of immunosuppressive adenosine. The ATP-rich tumor microenvironment presumably exasperates this accumulation by supplying substrate for NT5E. High level of adenosine is tumor protective through promotion of immune cell evasion and tumor cell growth and invasion. Indeed, in NT5E knockdown mice, primary tumor growth and metastasis was significantly reduced (Stagg, J. 2011, Jin, D. 2010, Zhi, X. 2010). Furthermore, NT5E targeted blocking by small molecule inhibitor AMPCP and anti-CD73 mAbs also reduced tumor growth in vivo and tumor cell proliferation, invasion, and migration in vitro (Bavaresco, L. 2008, Zhi, X. 2007, Stagg, J. 2011, Wang, L. 2011).

There exists a need for NT5E-targeting compositions and methods for use of such compositions in detection of NT5E and in treatment of conditions associated with NT5E such as cancer. Also recognized is a need for compositions and methods for differential inhibition of NT5E on cancer cells (e.g., in a tumor microenvironment) while leaving normal tissue unaffected.

### SUMMARY

To address certain needs in the art, the inventors have generated a diverse number of NT5E-binding proteins, including Fabs and antibodies. The disclosed potent inhibitory antibodies against NT5E may provide new strategies for therapeutic intervention in conditions such as cancer. Aspects of the disclosure relate to novel antibody and antigen binding fragments. Further aspects relate to polypeptides comprising the antigen binding fragment(s) of the disclosure, and compositions comprising the polypeptides, antibodies, and/or antigen binding fragments of the disclosure. Also described are nucleic acids encoding an antibody or antigen binding fragment of the disclosure. Further aspects relate to vectors or expression vectors comprising nucleic acids of the disclosure and host cells comprising polypeptides, nucleic acids, vectors, antibodies, or antigen binding fragments of the disclosure. The nucleic acids of the disclosure may be DNA or RNA.

Also described is a method of a making a cell comprising transferring one or more nucleic acid(s) of the disclosure into a cell. In some embodiments, the method further comprises culturing the cell under conditions that allow for expression of a polypeptide from the nucleic acid. In some embodiments, the method further comprising isolating the expressed polypeptide.

Further aspects of the disclosure relate to a method for treating or preventing cancer in a subject, the method comprising administering to the subject an antibody, antigen binding fragment, polypeptide, nucleic acid, or host cell of the disclosure. Yet further aspects relate to a method for evaluating a sample from a subject, the method comprising contacting a biological sample from the subject, or extract thereof, with at least one antibody, antigen binding fragment, or polypeptide of the disclosure. Also disclosed is a method for diagnosing cancer in a subject, the method comprising contacting a biological sample from the subject, or extract thereof, with at least one antibody, antigen binding fragment, or polypeptide of any one of the disclosure. In some embodiments, the antibodies, antigen binding fragments, or compositions of the disclosure are used to treat a subject having cancer.

Methods also provide for a method for evaluating a subject comprising administering to the subject an antibody, antigen binding fragment, polypeptide, composition, or cell of the disclosure. The antibody or antigen binding fragment may be conjugated to a radiolabel, which can be referred to as a radioisotope. The method may further comprises performing functional imaging on the subject. The functional imaging may comprise or exclude positron emission tomography (PET), single-photon emission computed tomography (SPECT), computed tomography (CT) imaging, magnetic resonance imaging (MRI), photoacoustic microscopy (PAM), magnetic particle imaging (MPI), optical imaging, scintigrapy, or combinations thereof.

Aspects of the disclosure relate to an antibody or antigen binding fragment comprising a heavy chain variable region and a light chain variable region, wherein the heavy chain variable region comprises a HCDR1, HCDR2, and HCDR3 from a heavy chain variable region of Table 1 and wherein the light chain variable region comprises a LCDR1, LCDR2, and LCDR3 from a light chain variable region of Table 1. Further aspects relate to an antibody or antigen binding fragment comprising a heavy chain variable region and a light chain variable region, wherein the heavy chain variable region comprises a HCDR1, HCDR2, and HCDR3 having at least 80% sequence identity or at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a HCDR1, HCDR2, and HCDR3 from a heavy chain variable region of Table 1 and wherein the light chain variable region comprises a LCDR1, LCDR2, and LCDR3 having at least 80% sequence identity or at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a LCDR1, LCDR2, and LCDR3 from a light chain variable region of Table 1. The HCDR1, HCDR2, HCDR3, LCDR1, LCDR2, and/or LCDR3 may be determined from the variable region sequences by methods known in the art. In some embodiments, the CDR is HCDR1, HCDR2, HCDR3, LCDR1, LCDR2, and/or LCDR3 determined by the Chothia method. In some embodiments, the CDR is HCDR1, HCDR2, HCDR3, LCDR1, LCDR2, and/or LCDR3 determined by the Kabat method. In some embodiments, the CDR is HCDR1, HCDR2, HCDR3, LCDR1, LCDR2, and/or LCDR3 determined by the IMGT method. In some embodiments, the antibody or antibody binding fragment comprises a heavy chain variable region and/or light chain variable region of Table 1 or a heavy chain variable region and/or light chain variable region having at least 80% sequence identity or at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a VH or VL of Table 1, such as a VH of one of SEQ ID NOs:7, 17, 47, 57, or 15 or a VL of one of SEQ ID NOs:8, 18, 58, or 23. In some embodiments, the antibody comprises a heavy chain and/or light chain of Table 1 or a heavy chain and/or light chain having at least 80% sequence identity or at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a heavy chain or light chain of Table 1, such as a heavy chain of one of SEQ ID NOs:9, 19, 29, 39, 49, 59, or 24, or a light chain of one of SEQ ID NOs:10, 20, 30, 60, or 25.

In certain embodiments, the heavy chain variable region and light chain variable region of Table 1 are from the same antibody clone of Table 1.

In some embodiments, the antibody or antigen binding fragment comprises a HCDR1 of one of SEQ ID NOs:1, 11, 21, or 5161.

In some embodiments, the antibody or antigen binding fragment comprises a HCDR2 of one of SEQ ID NOs:2, 22, 32, 42, 52, or 61.

In some embodiments, the antibody or antigen binding fragment comprises a HCDR3 of one of SEQ ID NOs:3, 13, 53, or 12.

In some embodiments, the antibody or antigen binding fragment comprises a LCDR1 of one of SEQ ID NO:4.

In some embodiments, the antibody or antigen binding fragment comprises a LCDR2 of one of SEQ ID NO:5.

In some embodiments, the antibody or antigen binding fragment comprises a LCDR3 of one of SEQ ID NOs:6, 16, 46, 56, or 14.

In some embodiments, the antibody or antigen binding fragment comprises a heavy chain variable region of one of SEQ ID NOs:7, 17, 27, 37, 47, 57, or 15.

In some embodiments, the antibody or antigen binding fragment comprises a light chain variable region of one of SEQ ID NOs:8, 18, 58, or 23.

An aspect of the disclosure relates to an antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:1-3, respectively, and wherein the light chain variable region comprises a LCDR3 comprising the amino acid sequence of SEQ ID NO:6.

An aspect of the disclosure relates to an antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:1-3, respectively, and wherein the light chain variable region comprises a LCDR1, LCDR2, and LCDR3 comprising the amino acid sequence of SEQ ID NOs:4-6, respectively.

An aspect of the disclosure relates to an antibody or antigen binding fragment comprising a heavy chain variable region and a light chain variable region, wherein the heavy chain variable region comprises a HCDR1, HCDR2, and HCDR3 having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NOs:1-3, respectively, and wherein the light chain variable region comprises a LCDR1, LCDR2, and LCDR3 having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NOs:4-6, respectively.

In some embodiments, the heavy chain variable region comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:7 and/or the light chain variable region comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:8.

In some embodiments, the antibody or antigen binding fragment comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOs:1-10 and/or an amino acid sequence with 1 substitution relative to SEQ ID NOs:1-10.

In some embodiments, the antibody comprises a heavy chain and a light chain and wherein the heavy chain comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:9 and the light chain comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:10.

An aspect of the disclosure relates to an antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:11, 2, and 13, respectively, and wherein the light chain variable region comprises a LCDR3 comprising the amino acid sequence of SEQ ID NO:16.

An aspect of the disclosure relates to an antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:11, 2, and 13, respectively, and wherein the light chain variable region comprises a LCDR1, LCDR2, and LCDR3 comprising the amino acid sequence of SEQ ID NOs:4, 5, and 16, respectively.

An aspect of the disclosure relates to an antibody or antigen binding fragment comprising a heavy chain variable region and a light chain variable region, wherein the heavy chain variable region comprises a HCDR1, HCDR2, and HCDR3 having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NOs:11, 2, and 13, respectively, and wherein the light chain variable region comprises a LCDR1, LCDR2, and LCDR3 having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NOs:4, 5, and 16, respectively.

In some embodiments, the heavy chain variable region comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:17 and/or the light chain variable region comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:18.

In some embodiments, the antibody or antigen binding fragment comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOs:11, 2, 13, 4, 5, and 16-20 and/or an amino acid sequence with 1 substitution relative to SEQ ID NOs:11, 2, 13, 4, 5, and 16-20.

In some embodiments, the antibody comprises a heavy chain and a light chain and wherein the heavy chain comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:19 and the light chain comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:20.

An aspect of the disclosure relates to an antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:21, 22, and 13, respectively, and wherein the light chain variable region comprises a LCDR3 comprising the amino acid sequence of SEQ ID NO:16.

An aspect of the disclosure relates to an antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:21, 22, and 13, respectively, and wherein the light chain variable region comprises a LCDR1, LCDR2, and LCDR3 comprising the amino acid sequence of SEQ ID NOs:4, 5, and 16, respectively.

An aspect of the disclosure relates to an antibody or antigen binding fragment comprising a heavy chain variable region and a light chain variable region, wherein the heavy chain variable region comprises a HCDR1, HCDR2, and HCDR3 having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NOs:21, 22, and 13, respectively, and wherein the light chain variable region comprises a LCDR1, LCDR2, and LCDR3 having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NOs:4, 5, and 16, respectively.

In some embodiments, the heavy chain variable region comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:27 and/or the light chain variable region comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:18.

In some embodiments, the antibody or antigen binding fragment comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOs:21, 22, 13, 4, 5, 16, 27, 18, 29, and 30 and/or an amino acid sequence with 1 substitution relative to SEQ ID NOs:21, 22, 13, 4, 5, 16, 27, 18, 29, and 30.

In some embodiments, the antibody comprises a heavy chain and a light chain and wherein the heavy chain comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:29 and the light chain comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:30.

An aspect of the disclosure relates to an antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:21, 32, and 13, respectively, and wherein the light chain variable region comprises a LCDR3 comprising the amino acid sequence of SEQ ID NO:16.

An aspect of the disclosure relates to an antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:21, 32, and 13, respectively, and wherein the light chain variable region comprises a LCDR1, LCDR2, and LCDR3 comprising the amino acid sequence of SEQ ID NOs:4, 5, and 16, respectively.

An aspect of the disclosure relates to an antibody or antigen binding fragment comprising a heavy chain variable region and a light chain variable region, wherein the heavy chain variable region comprises a HCDR1, HCDR2, and HCDR3 having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NOs:21, 32, and 13, respectively, and wherein the light chain variable region comprises a LCDR1, LCDR2, and LCDR3 having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NOs:4, 5, and 16, respectively.

In some embodiments, the heavy chain variable region comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:37 and/or the light chain variable region comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:18.

In some embodiments, the antibody or antigen binding fragment comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOs:21, 32, 13, 4, 5, 16, 37, 18, 39, and 30 and/or an amino acid sequence with 1 substitution relative to SEQ ID NOs:21, 32, 13, 4, 5, 16, 37, 18, 39, and 30.

In some embodiments, the antibody comprises a heavy chain and a light chain and wherein the heavy chain comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:39 and the light chain comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:30.

An aspect of the disclosure relates to an antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:21, 42, and 13, respectively, and wherein the light chain variable region comprises a LCDR3 comprising the amino acid sequence of SEQ ID NO:46.

An aspect of the disclosure relates to an antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:21, 42, and 13, respectively, and wherein the light chain variable region comprises a LCDR1, LCDR2, and LCDR3 comprising the amino acid sequence of SEQ ID NOs:4, 5, and 46, respectively.

An aspect of the disclosure relates to an antibody or antigen binding fragment comprising a heavy chain variable region and a light chain variable region, wherein the heavy chain variable region comprises a HCDR1, HCDR2, and HCDR3 having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NOs:21, 42, and 13, respectively, and wherein the light chain variable region comprises a LCDR1, LCDR2, and LCDR3 having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NOs:4, 5, and 46, respectively.

In some embodiments, the heavy chain variable region comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:47 and/or the light chain variable region comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:18.

In some embodiments, the antibody or antigen binding fragment comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOs:21, 42, 13, 4, 5, 46, 47, 18, 49, and 30 and/or an amino acid sequence with 1 substitution relative to SEQ ID NOs:21, 42, 13, 4, 5, 46, 47, 18, 49, and 30.

In some embodiments, the antibody comprises a heavy chain and a light chain and wherein the heavy chain comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:49 and the light chain comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:30.

An aspect of the disclosure relates to an antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:51-53, respectively, and wherein the light chain variable region comprises a LCDR3 comprising the amino acid sequence of SEQ ID NO:56.

An aspect of the disclosure relates to an antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:51-53, respectively, and wherein the light chain variable region comprises a LCDR1, LCDR2, and LCDR3 comprising the amino acid sequence of SEQ ID NOs:4, 5, and 56, respectively.

An aspect of the disclosure relates to an antibody or antigen binding fragment comprising a heavy chain variable region and a light chain variable region, wherein the heavy chain variable region comprises a HCDR1, HCDR2, and HCDR3 having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NOs:51-53, respectively, and wherein the light chain variable region comprises a LCDR1, LCDR2, and LCDR3 having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NOs:4, 5, and 56, respectively.

In some embodiments, the heavy chain variable region comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:57 and/or the light chain variable region comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:58.

In some embodiments, the antibody or antigen binding fragment comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOs:51-53, 4, 5, and 56-60 and/or an amino acid sequence with 1 substitution relative to SEQ ID NOs:51-53, 4, 5, and 56-60.

In some embodiments, the antibody comprises a heavy chain and a light chain and wherein the heavy chain comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:59 and the light chain comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:60.

An aspect of the disclosure relates to an antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:61, 62, and 12, respectively, and wherein the light chain variable region comprises a LCDR3 comprising the amino acid sequence of SEQ ID NO:14.

An aspect of the disclosure relates to an antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:61, 62, and 12, respectively, and wherein the light chain variable region comprises a LCDR1, LCDR2, and LCDR3 comprising the amino acid sequence of SEQ ID NOs:4, 5, and 14, respectively.

An aspect of the disclosure relates to an antibody or antigen binding fragment comprising a heavy chain variable region and a light chain variable region, wherein the heavy chain variable region comprises a HCDR1, HCDR2, and HCDR3 having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NOs:61, 62, and 12, respectively, and wherein the light chain variable region comprises a LCDR1, LCDR2, and LCDR3 having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NOs:4, 5, and 14, respectively.

In some embodiments, the heavy chain variable region comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:15 and/or the light chain variable region comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:23.

In some embodiments, the antibody or antigen binding fragment comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOs:61, 62, 12, 4, 5, 14, 15, 23, 24, or 25 and/or an amino acid sequence with 1 substitution relative to SEQ ID NOs: 61, 62, 12, 4, 5, 14, 15, 23, 24, or 25.

In some embodiments, the antibody comprises a heavy chain and a light chain and wherein the heavy chain comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:24 and the light chain comprises an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:25.

The antibody or antigen binding fragment of the disclosure may be human, chimeric, or humanized. The antibody or antigen binding fragment may be conjugated to a radiolabel. The radiolabel may be a therapeutic radioisotope. The radiolabel may be an alpha emitter, including for example actinium-225, radium-223, samarium-153, lead-212, bismuth-212, thorium-227. The radiolabel may be a beta minus emitter, including for example copper-67, lutetium-177, terbium-161. The radiolabel may be an Auger electron emitter, including for example indium-111, bromine-77, iodine-125. The radiolabel may comprise lutetium-177, actinium-225, radium-223, samarium-153, lead-212, bismuth-212, thorium-227, copper-67, terbium-161, indium-111, bromine-77, iodine-125, or a combination thereof. The radiolabel may comprise a radioisotope suitable for imaging, including PET, CT, and/or SPECT imaging. The radiolabel may comprise zirconium-89, copper-61, copper-64, fluorine-18, carbon-11, iodine-124, bromine-76, gallium-68, cerium-134, technetium-99, iodine-131, indium-111, lutetium-177, titanium-45, or a combination thereof. In some embodiments, the antibody, or antigen binding fragment binds NT5E with a K_{D} of about 10⁻⁶ M to about 10⁻¹² M. In some embodiments, the antibody or antigen binding fragment binds NT5E with a K_{D} of about, a K_{D} of at least, or a K_{D} of at most 10, 10⁻¹, 10⁻², 1⁻³, 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, 10⁻¹⁵, 10⁻¹⁶, 10⁻¹⁷, or 10⁻¹⁸ (or any derivable range therein) M, μM, nM, or pM. In some embodiments, the antibody or antigen binding fragment binds NT5E with a K_{D} of less than 20, 19, 18, 17, 16, 15, 14, 13, 12, 11, 10, 9, 8, 7, 6, 5, 4, 3, 2, or 1 nM, or any range or value derivable therein. In some embodiments, the antibody or antigen binding fragment binds NT5E with a K_{D} of less than 2 nM. In some embodiments, the antibody or antigen binding fragment binds NT5E with a K_{D} of less than 1 nM. In some embodiments, the antibody or antigen binding fragment binds NT5E with a K_{D} of less than 0.5 nM.

In some embodiments, the antibody or antigen binding fragment binds NT5E with a higher affinity in the presence of adenosine triphosphate (ATP) than in the absence of ATP. In some embodiments, the antibody or antigen binding fragment binds NT5E with at least, at most, or about a 1-, 2-, 3-, 4-, 5-, 6-, 7-, 8-, 9-, 10-, 15-, or 20-fold higher affinity in the presence of ATP than in the absence of ATP (or any range or value derivable therein). In some embodiments, the antibody or antigen binding fragment binds NT5E with a higher affinity in the presence of adenosine diphosphate (ADP) than in the absence of ADP. In some embodiments, the antibody or antigen binding fragment binds NT5E with at least, at most, or about a 1-, 2-, 3-, 4-, 5-, 6-, 7-, 8-, 9-, 10-, 15-, or 20-fold higher affinity in the presence of ADP than in the absence of ADP (or any range or value derivable therein).

In some embodiments, the antibody or antigen binding fragment is further defined as a human antibody or antigen binding fragment, humanized antibody or antigen binding fragment, recombinant antibody or antigen binding fragment, chimeric antibody or antigen binding fragment, an antibody or antigen binding fragment derivative, a veneered antibody or antigen binding fragment, a diabody, a monoclonal antibody or antigen binding fragment, a single domain antibody, or a single chain antibody. In some embodiments, the antigen binding fragment is further defined as a single chain variable fragment (scFv), F(ab′)₂, Fab′, Fab, Fv, or rIgG. In some embodiments, the antibody, antigen binding fragment, or polypeptide is operatively linked to a detectable label. Detectable labels are described herein.

Embodiments of the disclosure also relate to multi-specific antibodies and polypeptides. Accordingly, embodiments relate to bivalent or bispecific antibodies that comprise two antigen binding fragments, wherein the antigen binding fragment is two of the same antigen binding fragments or two different antigen binding fragments described herein. The disclosure also provides for multi-specific polypeptides. Embodiments relate to polypeptides comprising at least 2, 3, 4, 5, or 6 antigen binding fragments. The antigen binding fragment may be at least 2, 3, r, 5, or 6 scFv, F(ab′)₂, Fab′, Fab, Fv, or rIgG, or combinations thereof.

Compositions may comprise more than one antibody and/or antigen binding fragment of the disclosure. Accordingly, compositions of the disclosure may comprise, may comprise at least, or may comprise at most 1, 2, 3, 4, 5, 6, 7, 8, 9, or 10 antibodies and/or antigen binding fragments of the disclosure.

In some embodiments of the disclosure, the method further comprises incubating the antibody, antigen binding fragment, or polypeptide under conditions that allow for the binding of the antibody, antigen binding fragment, or polypeptide to antigens in the biological sample or extract thereof. In some embodiments, the method further comprises detecting the binding of an antigen to the antibody, antigen binding fragment, or polypeptide. In some embodiments, the method further comprises contacting the biological sample with at least one capture antibody, antigen, or polypeptide. The at least one capture antibody, antigen binding fragment, or polypeptide may be an antibody, polypeptide, or antigen binding fragment of the disclosure. In some embodiments, the capture antibody is linked or operatively linked to a solid support. The term “operatively linked” refers to a situation where two components are combined or capable of combining to form a complex. For example, the components may be covalently attached and/or on the same polypeptide, such as in a fusion protein or the components may have a certain degree of binding affinity for each other, such as a binding affinity that occurs through van der Waals forces. In some embodiments, the biological sample comprises a blood sample, urine sample, fecal sample, or nasopharyngeal sample. In embodiments of the disclosure, the at least one antibody, antigen binding fragment, or polypeptide may be operatively linked to a detectable label. In some embodiments, the method further comprises incubating the antibody, antigen binding fragment, or polypeptide under conditions that allow for the binding of the antibody, antigen binding fragment, or polypeptide to antigens in the biological sample or extract thereof. In some embodiments, the method further comprises detecting the binding of an antigen to the antibody, antigen binding fragment, or polypeptide. In some embodiments, the method further comprises contacting the biological sample with at least one capture antibody, antigen, or polypeptide. In some embodiments, the biological sample comprises a blood sample, serum sample, urine sample, fecal sample, or nasopharyngeal sample.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

“Individual, “subject,” and “patient” are used interchangeably and can refer to a human or non-human.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows SDS-PAGE of 1780 and 1789 IgG. The quality of 1780 and 1789 antibodies (3 μg) was assessed by SDS-polyacrylamide gel electrophoresis under non-reducing and reducing conditions (±5% β-mercaptoethanol (βME)).

FIGS. 2A-2C show 1789 Fab-mediated thermal stabilization of hNT5E in the presence of ATP as determined by differential scanning fluorimetry. Thermal shift assays of hNT5E_{WT} and two substrate binding knock-out variants, hNT5E_{DF} and hNT5E_{FF}, were conducted to highlight the dependence of 1789 Fab-NT5E stabilization on ATP binding. See Table 7 for a T_{M} summary. (FIG. 2A) hNT5E_{W}T in ATP-free condition (grey solid line), shows no change in thermal stability in the presence of 1789 Fab (grey dotted dashed line). With 1 mM ATP alone (red dashed line), the T_{M} shifts from one transition at 49° C. to two transitions at T_{M} 59° C. and 64° C. With both ATP and 1789 Fab (red dotted line), the thermal stability is fully shifted to T_{M} 66° C. Thermal shift assays of substrate binding knock-out variants hNT5E_{DF} (FIG. 2B) and hNT5E_{FF} (FIG. 2C) show no change in thermal stability with the addition of ATP. Without the stable binding of ATP to hNT5E_{DF} or hNT5E_{FF}, no additional stabilization is observed with 1789 Fab.

FIG. 3 shows the effect of NT5E pre-incubation with 1789 on ATP binding as measured by fluorescence polarization. Fluorescence polarization (FP) measures the dynamics of ATP binding in the presence of 1789 Fab and IgG. NT5E was incubated with either Fab or IgG prior to etheno-ATP (eATP) addition. FP signal was measured at 2 min intervals from eATP addition, t=0, to 90 min. Baseline was determined by the signal produced by eATP alone (yellow). FP signal produced by equilibrium binding of eATP is similar between NT5E alone (red) or in combination with BMS986179 IgG (purple). In contrast, with the addition of 1789 Fab (blue) or IgG (green), the signal increases. This effect corresponds to an enhanced stabilization of eATP to NT5E, presumably by 1789-NT5E interaction.

FIG. 4 shows in vitro 1789 binding to human, murine, and cyno NT5E by ELISA. Top panel from left to right: 1789 Fab binding to human, murine, and cyno NT5E. EC50_{ATP}=37±1 nM hNT5E; EC50_{ATP}=132±12 nM mNT5E; EC50_{ATP}=28±2 nM cNT5E. Bottom panel from left to right: 1789 IgG binding to human, murine, and cyno NT5E. EC50_{ATP}=0.86±0.03 nM and EC50_{APO}=2.14±1.23 nM hNT5E; EC50_{ATP}=1.14±0.03 nM mNT5E; and EC50_{ATP}=0.57±0.04 nM and EC50_{APO}=1.04±0.06 nM cNT5E. Data is represented as the mean±st. dev. of the Abs450 signal of three replicates. 1789 was cross-reactive between hNT5E, mNT5E, and cNT5E recombinant antigens.

FIG. 5 shows ATP-dependent binding of antibodies to human NT5E expressed on MDA-MB-231 cells by flow cytometry. Flow cytometry analysis of antibodies binding to human NT5E expressed on MDA-MB-231 cells in ATP-free condition (black) or in 1 mM ATP (red). Top panel: (left) 1780 Fab and (right) 1780 IgG titrations. Bottom panel: (left) 1789 Fab and (right) 1789 IgG titrations. Binding was measured at the indicated range of antibody concentrations and is expressed as mean±st. dev of median fluorescence intensity (MFI) of 1-2 replicates. Data were analyzed using OriginLab, and EC₅₀ values are shown next to the fitted curves. The ATP-free condition did not elicit sufficient fits, therefore only EC50s for the ATP condition are reported. In both Fab and IgG formats, 1789 exhibits distinctly improved binding in the presence of high ATP concentration.

FIG. 6 shows ATP-dependent binding of antibodies to murine NT5E expressed on 4T1 cells by flow cytometry. Flow cytometry analysis of 1789 IgG binding to human NT5E expressed on 4T1 cells in ATP-free conditions (black) or in 1 mM ATP (red). 1789 IgG binds preferentially to murine 4T1 cells in the presence of ATP with an EC50_{ATP}=37.3±3.6 nM.

FIG. 7 shows ATP-dependent binding and inhibitory properties of 1789 Fab on MDA-MB-231 cells. Left: 1789 Fab binding to MDA-MB-231 cells in the presence of multiple, fixed ATP concentrations. Data is represented as the mean±st. dev. of median fluorescence intensity (MFI) of three replicates. Right: Fab mediated inhibition of NT5E on MDA-MB-231 cells with multiple, fixed ATP concentrations. NT5E activity was initiated by addition of 200 μM AMP and final phosphate product was detected after 30 min. Data is represented as the mean st. dev. of normalized Relative % Activity. Titration of 1789 Fab in fixed [ATP] conditions in both binding and activity experiments on cells highlights the ATP-dependent properties of 1789.

FIG. 8 shows high nucleotide concentration-dependence of 1789 Fab binding to MDA-MB-231 cells. Flow cytometry analysis of 1789 Fab binding to MDA-MB-231 cells in the presence of multiple, fixed concentrations of nucleotides and nucleotide derivatives. Independent experiments were normalized using fixed concentration of Medi9447 IgG as a positive control. Data is represented as average±st. dev. of median fluorescence intensity (MFI) of two-three replicates. 1789 Fab preferentially bound to NT5E-expressing cells only in the presence of the high concentrations of nucleotides expected in the extracellular tumor microenvironment (>100 nM ATP denoted by the dotted line). In contrast, significantly less binding was detected for 1789 in conditions of low nucleotide concentration.

FIG. 9 shows ATP-dependent inhibition of NT5E-expressing MDA-MB-231 cells by 1780 and 1789 antibodies. MDA-MB-231 cells are pre-incubated with 500 μM ATP, Fab/IgG. Reaction was initialized with 200 μM AMP and after 30 min the phosphate product is measured using the EnzChek Phosphate Detection kit. Top panel: (left) 1780 Fab and (right) 1780 IgG inhibition of NT5E-mediated phosphate production on MDA-MB-231 cells. Bottom panel: (left) 1789 Fab and (right) 1789 IgG inhibition of NT5E-mediated phosphate production on MDA-MB-231 cells. Inhibition by Medi9447 and BMS986179 IgGs with ATP are overlaid in blue and purple, respectively. Data is representative of the mean±st. dev. of 4 6 replicates in 2⁻³ independent experiments. Normalized data is plotted and fit to a four-parameter logistic curve using OriginLab. No data fitting was possible for 1780 Fab or 1780 IgG. IC50 fits for 1789: 1789 Fab IC50_{ATP}=122±12 nM, and 1789 IgG IC50_{ATP}=2.2±0.2 nM. Note that the experimental conditions included 200 μM AMP, a moderately high concentration that may induce some 1789 IgG binding (see FIG. 8) and, subsequently, inhibition. In contrast to Medi9447 and BMS986179 IgG, 1789 IgG strongly inhibited NT5E with a maximal inhibition of 95%. Importantly, the distinct difference in 1789 IgG inhibition at 30 nM without ATP (max. 30%) and with ATP (max. 90%) supports the effectiveness of 1789 IgG at targeting NT5E specifically in the tumor microenvironment.

FIG. 10 shows ATP-dependent inhibition of 1789 IgG to murine NT5E expressed on 4T1 cells. 1789 IgG inhibition of NT5E-mediated phosphate production on 4T1 cells. Reaction is set up exactly as per MDA-MB-231 cells, see FIG. 9. Data could not be fitted to a four-parameter logistic curve and is represented as data points alone. 1789 IgG exhibits ATP-sensitive inhibition on 4T1 cells.

FIG. 11 shows affinity matured 1789 variants eliciting ATP-dependent NT5E inhibition on human and murine cell lines. Three variants were chosen to demonstrate the improved inhibitory properties of 1789 through affinity maturation. Reactions were set up and data analyzed as described for FIG. 9. Left: Fab and IgG inhibition of NT5E activity on human MDA-MB-231 cells. IC50 fits for 1789 variants: IgG32 IC50_{NoATP}=5.6±0.3 nM and IC50_{ATP}=2.3±0.2 nM; IgG39 IC50_{NoATP}=6.9±0.4 nM and IC50_{ATP}=2.5±0.1 nM; IgG42 IC50_{NoATP}=5.4 0.8 nM and IC50_{ATP}=1.9±0.2 nM. Right: Fab and IgG inhibition of NT5E activity on murine 4T1 cells. IC50 fits for 1789 variants: IgG32 IC50_{ATP}=9.1±0.3 nM; IgG39 IC50_{ATP}=12.7±0.6 nM; IgG42 IC50_{ATP}=8.8±1.0 nM. In all graphs, Fab data in No ATP and ATP conditions is in black and red, respectively, and IgG data in No ATP and ATP conditions is in blue and green, respectively. MDA-MB-231 Fab/IgG and 4T1 Fab data is representative of one experiment, while 4T1 IgG data is the mean±st. dev. of 2 independent experiments. Murine No ATP condition data could not be fitted therefore parameters are not reported. The limited murine cross-reactivity of 1789 is overcome by affinity maturation as 1789 variants show significantly improved potent and efficacious ATP-sensitive inhibition of murine NT5E.

FIG. 12 shows a comparison of cell-based inhibitory properties of Medi9447 and BMS986179 IgGs with 1789 IgG. Left: Unlike 1789 IgG, antibodies Medi9447 and BMS986179 did not exhibit ATP-dependent inhibition of MDA-MB-231 cells. Medi9447 IgG IC50 fits: IC50_{ATP}=1.0±0.3 nM and IC50_{NoATP}=0.3±0.1 nM; BMS986179 IgG IC50 fits: IC50_{ATP}=1.1±0.1 nM and IC50_{NoATP}=0.6±0.1 nM. Right: Maximal inhibition measured for BMS986179 was approximately 70% while 1789 IgG achieved a maximal inhibition of 95%. 1789 IgG maintained strong NT5E inhibition at high IgG concentrations without the Hook effect. Thus, under the specific conditions of the tumor microenvironment, 1789 IgG was more efficacious than both commercial antibodies tested with similar potency with respect to inhibition of NT5E on cells.

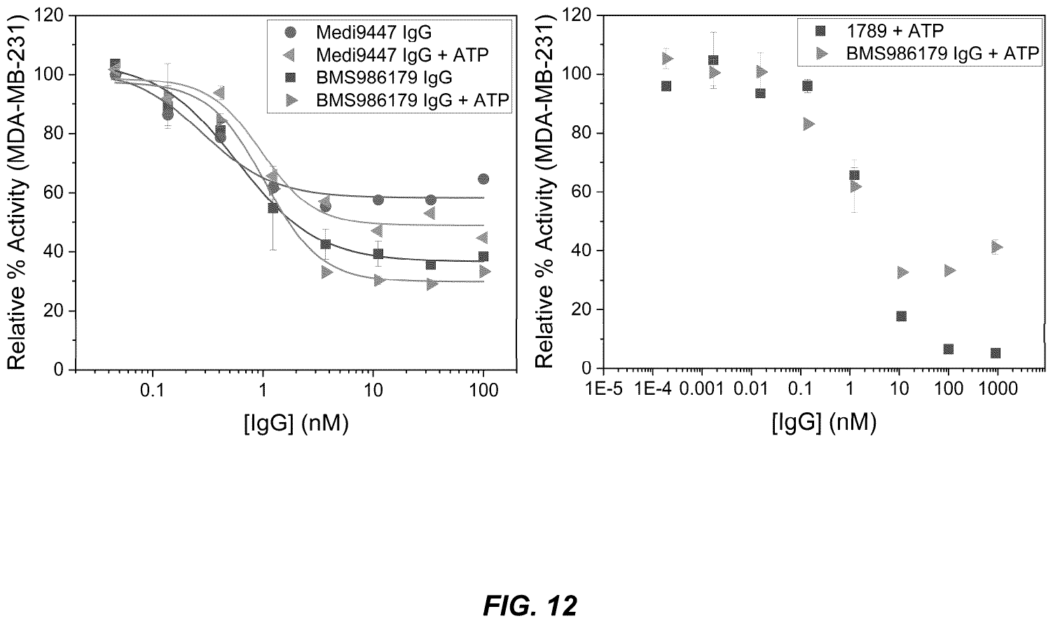

FIG. 13 shows epitope binning of 1789 by sandwich ELISA. Left: Epitope binning of 1789 by sandwich ELISA with benchmark IgGs. Medi9447 and BMS986179 IgGs were immobilized onto an ELISA plate and incubated with either NT5E alone or NT5E mixed with 1 μM 1789 Fab with constant high [ATP]. After incubation, the remaining NT5E is probed with αStrep-HRP Ab. If there is competition for binding to NT5E, then a decrease in signal would be expected. Since no change in signal was observed, 1789 does not share an epitope footprint with Medi9447 or BMS986179 IgGs. Right: SPR-mediated epitope binning of 1789 against RAN “Medi9447-like” 1557 Fab and BMS986179 Fab. Human NT5E-6×His was first immobilized to an NTA sensor chip then primary Fab was injected followed by co-injection of a mixture of both primary Fab (control) and secondary Fab (1789). An increase in ARU upon co-injection with 1789 indicates a unique epitope compared to N-terminal binders 1557 and BMS986179.

FIG. 14 shows interdomain binding of 1789. Shown are SPR traces of 1789 Fab binding to full-length hNT5E (red line), N-terminal domain (blue line), and C-terminal domain (green). 1789 Fab requires both the N- and C-terminal domains to bind effectively to NT5E.

FIG. 15. Radio-iTLC of purified 177Lu-DOTA-AM39. Mobile phase: 20 mM citric acid.

FIG. 16. Radio-iTLC of purified ¹⁷⁷Lu-DOTA-1798. Mobile phase: 20 mM citric acid.

FIG. 17. ⁸⁹Zr-AM39 (radiolabeled NT5E antibody) specifically localizes to 4T1 tumors in vivo.

FIG. 18. AM39 has high tumor uptake with low background.

FIG. 19. Biodistribution studies of ¹⁷⁷Lu-1789 and ¹⁷⁷Lu-AM39.

FIG. 20. Antitumor assessment study (4T1 tumors). At right is shown tumor volume data from an antitumor assessment study using 177Lu-AM39. Female balb/c mice bearing subcutaneous 4T1 tumors (n=8) were injected with 177Lu-AM39 intravenously on day 0 and day 6 of the study. Mice received ˜300 uCi per injection. Tumor growth was significantly inhibited by drug treatment starting from day 8. At right are depicted the tumor volume and normalized tumor volume values on day 15. 177Lu-AM39 significantly inhibited tumor growth. *P<0.01

FIG. 21. Biodistribution of ⁸⁹Zr-AM39 and ⁸⁹Zr-1798. At left is shown transaxial and coronal PET/CT slices and three dimensional maximum intensity projections of female balb/c mice bearing subcutaneous 4T1 tumors. The site of the tumor is indicated with an orange arrow. At right is shown region of interest analysis from the mice in each cohort (n=4).

FIG. 22. Tumor uptake of ⁸⁹Zr-AM39 and ⁸⁹Zr-1798. At left is shown coronal slices from representative male immunocompetent mice bearing subcutaneous MC38 or RM1 tumors. The imaging data were collected at 96 hour post injection. At right is shown the SUVmean of tumors in each cohort (n=4).

FIGS. 23A and 23B. Cryo-EM structure of the Fab32-NT5E complex (FIG. 23A) Structure of Fab32 variable domains bound to the NT5E dimer in closed state. Variable domains of Fabs are colored in dark slate blue and dark red. NT5E dimer is colored in sea green. AMPCP molecule and zinc ions at the active site are shown as cylinders and balls, respectively; (FIG. 23B) AMPCP binding pocket. AMPCP and NT5E residues are shown as cylinders in green and ice blue, respectively. Non-carbon atoms are colored by type: oxygen in red, nitrogen in blue, and sulfur in pink. Zink ions are shown as balls in grey color.

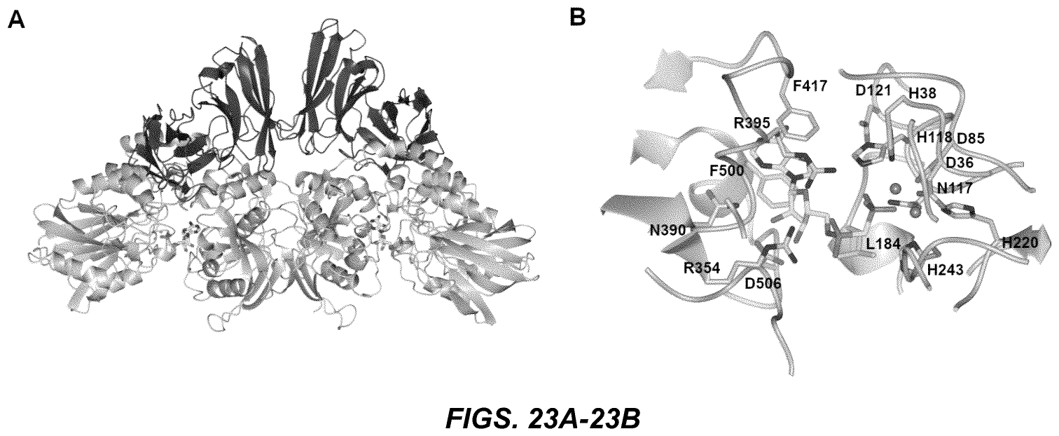

### DETAILED DESCRIPTION OF THE INVENTION

Targeting the NT5E-adenosine axis is a promising pathway for cancer immunotherapy. Accumulation of NT5E-generated adenosine in the extracellular milieu forms a tumor protective, immuno-suppressive microenvironment. In addition to dampening the immune response, prolonged adenosine stimulation promotes tumor cell invasion and metastasis. Adenosine blockade by anti-NT5E immunotherapy aims to alleviate these effects and re-activate the anti-tumor immunity. As disclosed herein, antibody phage display was used to generate antibody fragments (FABs) and antibodies (IgGs) that bind to NT5E, including those whose NT5E affinity is ATP-dependent. Accordingly, disclosed are compositions comprising various targeting molecules (e.g., antibodies, antibody fragments, antibody-like molecules, antibody-drug conjugates, chimeric antigen receptors, BiTES, etc.) capable of targeting NT5E, as well as methods for use of such molecules for detection, diagnosis, and treatment of conditions associated with NT5E, such as cancer.

### I. ANTIBODIES

Aspects of the disclosure relate to antibodies, antigen binding fragments thereof, or polypeptides capable of specifically binding to NT5E (CD73).

The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986). Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4: 302; 2013).

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the COOH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

### A. Types of Antibodies

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins.

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-L1 (or LCDR1), CDR-L2 (or LCDR2), and CDR-L3 (or LCDR3). The L3 (CDR-L3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as CDR-H1 (or HCDR1), CDR-H2 (or HCDR2), and CDR-H3 (or HCDR3). The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55⁻⁷⁷, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include:
1) Computational predictions of the tertiary structure of the antibody/epitope binding interactions based on the chemical nature of the amino acid sequence of the antibody variable region and composition of the epitope.
2) Hydrogen-deuterium exchange and mass spectroscopy
3) Polypeptide fragmentation and peptide mapping approaches in which one generates multiple overlapping peptide fragments from the full length of the polypeptide and evaluates the binding affinity of these peptides for the epitope.
4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24: 8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some embodiments, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

### B. Functional Antibody Fragments and Antigen-Binding Fragments

### 1. Antigen-Binding Fragments

Certain aspects relate to antibody fragments, such as antibody fragments that bind to NT5E. The term functional antibody fragment includes antigen-binding fragments of an antibody that retain the ability to specifically bind to an antigen. These fragments are constituted of various arrangements of the variable region heavy chain (VH) and/or light chain (VL); and in some embodiments, include constant region heavy chain 1 (CH1) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, N Y (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015), each of which are incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment (also “Fab” or “FAB”) means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

### 2. Fragment Antigen Binding Region, Fab

Fab polypeptides of the disclosure include the Fab antigen binding fragment of an antibody. Unless specifically stated otherwise, the term “Fab” relates to a polypeptide excluding the Fc portion of the antibody. The Fab may be conjugated to a polypeptide comprising other components, such as further antigen binding domains, costimulatory domains, linkers, peptide spacers, transmembrane domains, endodomains, and accessory proteins. Fab polypeptides can be generated using conventional techniques known in the art and are well-described in the literature.

### 3. Fragment Crystallizable Region, Fe

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

C. Polypeptides with antibody CDRs & Scaffolding Domains that Display the CDRs

Antigen-binding peptide scaffolds, such as complementarity-determining regions (CDRs), are used to generate protein-binding molecules in accordance with the embodiments. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

### D. Antibody Binding

The term “selective binding agent” refers to a molecule that binds to an antigen. Non-limiting examples include antibodies, antigen-binding fragments, scFv, Fab, Fab′, F(ab′)2, single chain antibodies, peptides, peptide fragments and proteins.

The term “binding” refers to a direct association between two molecules, due to, for example, covalent, electrostatic, hydrophobic, and ionic and/or hydrogen-bond interactions, including interactions such as salt bridges and water bridges. “Immunologically reactive” means that the selective binding agent or antibody of interest will bind with antigens present in a biological sample. The term “immune complex” refers the combination formed when an antibody or selective binding agent binds to an epitope on an antigen.

### 1. Affinity/Avidity

The term “affinity” refers the strength with which an antibody or selective binding agent binds an epitope. In antibody binding reactions, this is expressed as the affinity constant (Ka or ka sometimes referred to as the association constant) for any given antibody or selective binding agent. Affinity is measured as a comparison of the binding strength of the antibody to its antigen relative to the binding strength of the antibody to an unrelated amino acid sequence. Affinity can be expressed as, for example, 20-fold greater binding ability of the antibody to its antigen then to an unrelated amino acid sequence. As used herein, the term “avidity” refers to the resistance of a complex of two or more agents to dissociation after dilution. The terms “immunoreactive” and “preferentially binds” are used interchangeably herein with respect to antibodies and/or selective binding agent.

There are several experimental methods that can be used by one skilled in the art to evaluate the binding affinity of any given antibody or selective binding agent for its antigen. This is generally done by measuring the equilibrium dissociation constant (KD or Kd), using the equation KD=koff/kon=[A] [B]/[AB]. The term koff is the rate of dissociation between the antibody and antigen per unit time, and is related to the concentration of antibody and antigen present in solution in the unbound form at equilibrium. The term kon is the rate of antibody and antigen association per unit time, and is related to the concentration of the bound antigen-antibody complex at equilibrium. The units used for measuring the KD are mol/L (molarity, or M), or concentration. The Ka of an antibody is the opposite of the KD, and is determined by the equation Ka=1/KD. Examples of some experimental methods that can be used to determine the KD value are: enzyme-linked immunosorbent assays (ELISA), isothermal titration calorimetry (ITC), fluorescence anisotropy, surface plasmon resonance (SPR), and affinity capillary electrophoresis (ACE). The affinity constant (Ka) of an antibody is the opposite of the KD, and is determined by the equation Ka=1/KD.

Antibodies deemed useful in certain embodiments may have an affinity constant (Ka) of about, at least about, or at most about 10⁶, 10⁷, 10⁸, 10⁹, or 10¹⁰ M or any range derivable therein. Similarly, in some embodiments, antibodies may have a dissociation constant of about, at least about or at most about 10⁻⁶, 10⁻⁷, 10⁻⁸, 10⁻⁹, 10⁻¹⁰ M, or any range derivable therein. These values are reported for antibodies discussed herein and the same assay may be used to evaluate the binding properties of such antibodies. An antibody of the invention is said to “specifically bind” its target antigen when the dissociation constant (KD) is ≤10⁻⁸ M. The antibody specifically binds antigen with “high affinity” when the KD is ≤5×10⁻⁹ M, and with “very high affinity” when the KD is ≤5×10⁻¹⁰ M.

### 2. Epitope Specificity

The epitope of an antigen is the specific region of the antigen for which an antibody has binding affinity. In the case of protein or polypeptide antigens, the epitope is the specific residues (or specified amino acids or protein segment) that the antibody binds with high affinity. An antibody does not necessarily contact every residue within the protein. Nor does every single amino acid substitution or deletion within a protein necessarily affect binding affinity. For purposes of this specification and the accompanying claims, the terms “epitope” and “antigenic determinant” are used interchangeably to refer to the site on an antigen to which B and/or T cells respond or recognize. Polypeptide epitopes can be formed from both contiguous amino acids and noncontiguous amino acids juxtaposed by tertiary folding of a polypeptide. An epitope typically includes at least 3, and typically 5-10 amino acids in a unique spatial conformation.

Epitope specificity of an antibody can be determined in a variety of ways. One approach, for example, involves testing a collection of overlapping peptides of 15 amino acids spanning the full sequence of the protein and differing in increments of a small number of amino acids (e.g., 3 to 30 amino acids). The peptides are immobilized in separate wells of a microtiter dish. Immobilization can be accomplished, for example, by biotinylating one terminus of the peptides. This process may affect the antibody affinity for the epitope, therefore different samples of the same peptide can be biotinylated at the N and C terminus and immobilized in separate wells for the purposes of comparison. This is useful for identifying end-specific antibodies. Optionally, additional peptides can be included terminating at a particular amino acid of interest. This approach is useful for identifying end-specific antibodies to internal fragments. An antibody or antigen-binding fragment is screened for binding to each of the various peptides. The epitope is defined as a segment of amino acids that is common to all peptides to which the antibody shows high affinity binding.

### 3. Modification of Antibody Antigen-Binding Domains

It is understood that the antibodies of the present invention may be modified, such that they are substantially identical to the antibody polypeptide sequences, or fragments thereof, and still bind the epitopes of the present invention. Polypeptide sequences are “substantially identical” when optimally aligned using such programs as Clustal Omega, IGBLAST, GAP or BESTFIT using default gap weights, they share at least 80% sequence identity, at least 90% sequence identity, at least 95% sequence identity, at least 96% sequence identity, at least 97% sequence identity, at least 98% sequence identity, or at least 99% sequence identity or any range therein.

As discussed herein, minor variations in the amino acid sequences of antibodies or antigen-binding regions thereof are contemplated as being encompassed by the present invention, providing that the variations in the amino acid sequence maintain at least 75%, more preferably at least 80%, at least 90%, at least 95%, at least 96%, at least 97%, at least 98% and most preferably at least 99% sequence identity. In particular, conservative amino acid replacements are contemplated.

Conservative replacements are those that take place within a family of amino acids that are related in their side chains. Genetically encoded amino acids are generally divided into families based on the chemical nature of the side chain; e.g., acidic (aspartate, glutamate), basic (lysine, arginine, histidine), nonpolar (alanine, valine, leucine, isoleucine, proline, phenylalanine, methionine, tryptophan), and uncharged polar (glycine, asparagine, glutamine, cysteine, serine, threonine, tyrosine). For example, it is reasonable to expect that an isolated replacement of a leucine moiety with an isoleucine or valine moiety, or a similar replacement of an amino acid with a structurally related amino acid in the same family, will not have a major effect on the binding or properties of the resulting molecule, especially if the replacement does not involve an amino acid within a framework site. Whether an amino acid change results in a functional peptide can readily be determined by assaying the specific activity of the polypeptide derivative. Standard ELISA, Surface Plasmon Resonance (SPR), or other antibody binding assays can be performed by one skilled in the art to make a quantitative comparison of antigen binging affinity between the unmodified antibody and any polypeptide derivatives with conservative substitutions generated through any of several methods available to one skilled in the art.

Fragments or analogs of antibodies or immunoglobulin molecules can be readily prepared by those skilled in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Standard methods to identify protein sequences that fold into a known three-dimensional structure are available to those skilled in the art; Dill and McCallum., Science 338:1042-1046 (2012). Several algorithms for predicting protein structures and the gene sequences that encode these have been developed, and many of these algorithms can be found at the National Center for Biotechnology Information (on the World Wide Web at ncbi.nlm.nih.gov/guide/proteins/) and at the Bioinformatics Resource Portal (on the World Wide Web at expasy.org/proteomics). Thus, the foregoing examples demonstrate that those of skill in the art can recognize sequence motifs and structural conformations that may be used to define structural and functional domains in accordance with the invention.

Framework modifications can be made to antibodies to decrease immunogenicity, for example, by “backmutating” one or more framework residues to a corresponding germline sequence.

It is also contemplated that the antigen-binding domain may be multi-specific or multivalent by multimerizing the antigen-binding domain with VH and VL region pairs that bind either the same antigen (multi-valent) or a different antigen (multi-specific).

### E. Chemical Modification of Antibodies

In some aspects, also contemplated are glycosylation variants of antibodies, wherein the number and/or type of glycosylation site(s) has been altered compared to the amino acid sequences of the parent polypeptide. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861). In certain embodiments, antibody protein variants comprise a greater or a lesser number of N-linked glycosylation sites than the native antibody. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. In other embodiments, one or more new N-linked glycosylation sites are created. Antibodies typically have an N-linked glycosylation site in the Fc region.

Additional antibody variants include cysteine variants, wherein one or more cysteine residues in the parent or native amino acid sequence are deleted from or substituted with another amino acid (e.g., serine). Cysteine variants are useful, inter alia, when antibodies must be refolded into a biologically active conformation. Cysteine variants may have fewer cysteine residues than the native antibody and typically have an even number to minimize interactions resulting from unpaired cysteines.

In some aspects, the polypeptides can be pegylated to increase biological half-life by reacting the polypeptide with polyethylene glycol (PEG) or a reactive ester or aldehyde derivative of PEG, under conditions in which one or more PEG groups become attached to the polypeptide. Polypeptide pegylation may be carried out by an acylation reaction or an alkylation reaction with a reactive PEG molecule (or an analogous reactive water-soluble polymer). Methods for pegylating proteins are known in the art and can be applied to the polypeptides of the invention to obtain PEGylated derivatives of antibodies. See, e.g., EP 0 154 316 and EP 0 401 384. In some aspects, the antibody is conjugated or otherwise linked to transthyretin (TTR) or a TTR variant. The TTR or TTR variant can be chemically modified with, for example, a chemical selected from the group consisting of dextran, poly(n-vinyl pyrrolidone), polyethylene glycols, propropylene glycol homopolymers, polypropylene oxide/ethylene oxide co-polymers, polyoxyethylated polyols, and polyvinyl alcohols. As used herein, the term “polyethylene glycol” is intended to encompass any of the forms of PEG that have been used to derivatize other proteins.

### 1. Conjugation

Derivatives of the antibodies and antigen binding fragments that are described herein are also provided. The derivatized antibody or fragment thereof may comprise any molecule or substance that imparts a desired property to the antibody or fragment. The derivatized antibody can comprise, for example, a detectable (or labeling) moiety (e.g., a radioactive, colorimetric, antigenic, or enzymatic molecule, or a detectable bead), a molecule that binds to another molecule (e.g., biotin or streptavidin), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses).

Optionally, an antibody or an immunological portion of an antibody can be chemically conjugated to, or expressed as, a fusion protein with other proteins. In some aspects, polypeptides may be chemically modified by conjugating or fusing the polypeptide to serum protein, such as human serum albumin, to increase half-life of the resulting molecule. See, e.g., EP 0322094 and EP 0 486 525. In some aspects, the polypeptides may be conjugated to a diagnostic agent and used diagnostically, for example, to monitor the development or progression of a disease and determine the efficacy of a given treatment regimen. In some aspects, the polypeptides may also be conjugated to a therapeutic agent to provide a therapy in combination with the therapeutic effect of the polypeptide. Additional suitable conjugated molecules include ribonuclease (RNase), DNase I, an antisense nucleic acid, an inhibitory RNA molecule such as a siRNA molecule, an immunostimulatory nucleic acid, aptamers, ribozymes, triplex forming molecules, and external guide sequences. The functional nucleic acid molecules may act as effectors, inhibitors, modulators, and stimulators of a specific activity possessed by a target molecule, or the functional nucleic acid molecules may possess a de novo activity independent of any other molecules.

In some aspects, disclosed are antibodies and antibody-like molecules that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules include toxins, therapeutic enzymes, antibiotics, radiolabeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles, or ligands.

a. Conjugate Types

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to be detected, and/or further quantified if desired. Examples of detectable labels include, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. Particular examples of labels are, but not limited to, horseradish peroxidase (HRP), fluorescein, FITC, rhodamine, dansyl, umbelliferone, dimethyl acridinium ester (DMAE), Texas red, luminol, NADPH and α- or β-galactosidase. Antibody conjugates include those intended primarily for use in vitro, where the antibody is linked to a secondary binding ligand and/or to an enzyme to generate a colored product upon contact with a chromogenic substrate. Examples of suitable enzymes include, but are not limited to, urease, alkaline phosphatase, (horseradish) hydrogen peroxidase, or glucose oxidase. Preferred secondary binding ligands are biotin and/or avidin and streptavidin compounds. The uses of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference. Molecules containing azido groups may also be used to form covalent bonds to proteins through reactive nitrene intermediates that are generated by low intensity ultraviolet light (Potter & Haley, 1983).

In some aspects, contemplated are immunoconjugates comprising an antibody or antigen-binding fragment thereof conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). In this way, the agent of interest can be targeted directly to cells bearing cell surface antigen. The antibody and agent may be associated through non-covalent interactions such as through electrostatic forces, or by covalent bonds. Various linkers, known in the art, can be employed in order to form the immunoconjugate. Additionally, the immunoconjugate can be provided in the form of a fusion protein. In one aspect, an antibody may be conjugated to various therapeutic substances in order to target the cell surface antigen. Examples of conjugated agents include, but are not limited to, metal chelate complexes, drugs, toxins and other effector molecules, such as cytokines, lymphokines, chemokines, immunomodulators, radiosensitizers, asparaginase, carboranes, and radioactive halogens.

In antibody drug conjugates (ADC), an antibody (Ab) is conjugated to one or more drug moieties (D) through a linker (L). The ADC may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody. Antibody drug conjugates may also be produced by modification of the antibody to introduce electrophilic moieties, which can react with nucleophilic substituents on the linker reagent or drug. Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate. In yet another aspect, the antibody may be conjugated to a “receptor” (such as streptavidin) for utilization in tumor or cancer cell pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

Examples of an antibody-drug conjugates known to a person skilled in the art are pro-drugs useful for the local delivery of cytotoxic or cytostatic agents, i.e. drugs to kill or inhibit tumor cells in the treatment of cancer (Syrigos and Epenetos, Anticancer Res. 19:605-614 (1999); Niculescu-Duvaz and Springer, Adv. Drg. Del. Rev. 26:151-172 (1997); U.S. Pat. No. 4,975,278). In contrast, systematic administration of these unconjugated drug agents may result in unacceptable levels of toxicity to normal cells as well as the target tumor cells (Baldwin et al., Lancet 1:603-5 (1986); Thorpe, (1985) “Antibody Carriers of Cytotoxic Agents in Cancer Therapy: A Review,” In: Monoclonal Antibodies '84: Biological and Clinical Applications, A. Pincera et al., (eds.) pp. 475-506). Both polyclonal antibodies and monoclonal antibodies have been reported as useful in these strategies (Rowland et al., Cancer Immunol. Immunother. 21:183-87 (1986)).

In certain aspects, ADC include covalent or aggregative conjugates of antibodies, or antigen-binding fragments thereof, with other proteins or polypeptides, such as by expression of recombinant fusion proteins comprising heterologous polypeptides fused to the N-terminus or C-terminus of an antibody polypeptide. For example, the conjugated peptide may be a heterologous signal (or leader) polypeptide, e.g., the yeast alpha-factor leader, or a peptide such as an epitope tag (e.g., V5-His). Antibody-containing fusion proteins may comprise peptides added to facilitate purification or identification of the antibody (e.g., poly-His). An antibody polypeptide also can be linked to the FLAG® (Sigma-Aldrich, St. Louis, Mo.) peptide as described in Hopp et al., Bio/Technology 6:1204 (1988), and U.S. Pat. No. 5,011,912. Oligomers that contain one or more antibody polypeptides may be employed as antagonists. Oligomers may be in the form of covalently linked or non-covalently linked dimers, trimers, or higher oligomers. Oligomers comprising two or more antibody polypeptides are contemplated for use. Other oligomers include heterodimers, homotrimers, heterotrimers, homotetramers, heterotetramers, etc. In certain aspects, oligomers comprise multiple antibody polypeptides joined via covalent or non-covalent interactions between peptide moieties fused to the antibody polypeptides. Such peptides may be peptide linkers (spacers), or peptides that have the property of promoting oligomerization. Leucine zippers and certain polypeptides derived from antibodies are among the peptides that can promote oligomerization of antibody polypeptides attached thereto, as described in more detail below.

b. Conjugation Methodology

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates may also be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bos(p-diazoniumbenzoyl)-ethylenediamine), diisocyanates (such as toluene 2,6-diisocyanate), and bis-active fluorine compounds (such as 1,5-difluoro-2,4-dinitrobenzene). In some aspects, derivatization of immunoglobulins by selectively introducing sulfhydryl groups in the Fc region of an immunoglobulin, using reaction conditions that do not alter the antibody combining site, are contemplated. Antibody conjugates produced according to this methodology are disclosed to exhibit improved longevity, specificity, and sensitivity (U.S. Pat. No. 5,196,066, incorporated herein by reference). Site-specific attachment of effector or reporter molecules, wherein the reporter or effector molecule is conjugated to a carbohydrate residue in the Fc region has also been disclosed in the literature (O'Shannessy et al., 1987).

### II. ANTIBODY PRODUCTION

### A. Antibody Production

Methods for preparing and characterizing antibodies for use in diagnostic and detection assays, for purification, and for use as therapeutics are well known in the art as disclosed in, for example, U.S. Pat. Nos. 4,011,308; 4,722,890; 4,016,043; 3,876,504; 3,770,380; and 4,372,745 (see, e.g., Antibodies: A Laboratory Manual, Cold Spring Harbor Laboratory, 1988; incorporated herein by reference). These antibodies may be polyclonal or monoclonal antibody preparations, monospecific antisera, human antibodies, hybrid or chimeric antibodies, such as humanized antibodies, altered antibodies, F(ab′)2 fragments, Fab fragments, Fv fragments, single-domain antibodies, dimeric or trimeric antibody fragment constructs, minibodies, or functional fragments thereof which bind to the antigen in question. In certain aspects, polypeptides, peptides, and proteins and immunogenic fragments thereof for use in various embodiments can also be synthesized in solution or on a solid support in accordance with conventional techniques. See, for example, Stewart and Young, (1984); Tarn et al, (1983); Merrifield, (1986); and Barany and Merrifield (1979), each incorporated herein by reference.

Briefly, a polyclonal antibody is prepared by immunizing an animal with an antigen or a portion thereof and collecting antisera from that immunized animal. The antigen may be altered compared to an antigen sequence found in nature. In some embodiments, a variant or altered antigenic peptide or polypeptide is employed to generate antibodies. Inocula are typically prepared by dispersing the antigenic composition in a physiologically tolerable diluent to form an aqueous composition. Antisera is subsequently collected by methods known in the arts, and the serum may be used as-is for various applications or else the desired antibody fraction may be purified by well-known methods, such as affinity chromatography (Harlow and Lane, Antibodies: A Laboratory Manual 1988).

Methods of making monoclonal antibodies are also well known in the art (Kohler and Milstein, 1975; Harlow and Lane, 1988, U.S. Pat. No. 4,196,265, herein incorporated by reference in its entirety for all purposes). Typically, this technique involves immunizing a suitable animal with a selected immunogenic composition, e.g., a purified or partially purified protein, polypeptide, peptide or domain. Resulting antibody-producing B-cells from the immunized animal, or all dissociated splenocytes, are then induced to fuse with cells from an immortalized cell line to form hybridomas. Myeloma cell lines suited for use in hybridoma-producing fusion procedures preferably are non-antibody-producing and have high fusion efficiency and enzyme deficiencies that render then incapable of growing in certain selective media that support the growth of only the desired fused cells (hybridomas). Typically, the fusion partner includes a property that allows selection of the resulting hybridomas using specific media. For example, fusion partners can be hypoxanthine/aminopterin/thymidine (HAT)-sensitive. Methods for generating hybrids of antibody-producing spleen or lymph node cells and myeloma cells usually comprise mixing somatic cells with myeloma cells in the presence of an agent or agents (chemical or electrical) that promote the fusion of cell membranes. Next, selection of hybridomas can be performed by culturing the cells by single-clone dilution in microtiter plates, followed by testing the individual clonal supernatants (after two to three weeks) for the desired reactivity. Fusion procedures for making hybridomas, immunization protocols, and techniques for isolation of immunized splenocytes for fusion are known in the art.

Other techniques for producing monoclonal antibodies include the viral or oncogenic transformation of B-lymphocytes, a molecular cloning approach may be used to generate a nucleic acid or polypeptide, the selected lymphocyte antibody method (SLAM) (see, e.g., Babcook et al., Proc. Natl. Acad. Sci. USA 93:7843-7848 (1996), the preparation of combinatorial immunoglobulin phagemid libraries from RNA isolated from the spleen of the immunized animal and selection of phagemids expressing appropriate antibodies, or producing a cell expressing an antibody from a genomic sequence of the cell comprising a modified immunoglobulin locus using Cre-mediated site-specific recombination (see, e.g., U.S. Pat. No. 6,091,001).

Monoclonal antibodies may be further purified using filtration, centrifugation, and various chromatographic methods such as HPLC or affinity chromatography. Monoclonal antibodies may be further screened or optimized for properties relating to specificity, avidity, half-life, immunogenicity, binding association, binding disassociation, or overall functional properties relative to being a treatment for infection. Thus, monoclonal antibodies may have alterations in the amino acid sequence of CDRs, including insertions, deletions, or substitutions with a conserved or non-conserved amino acid.

The immunogenicity of a particular immunogen composition can be enhanced by the use of non-specific stimulators of the immune response, known as adjuvants. Adjuvants that may be used in accordance with embodiments include, but are not limited to, IL-1, IL-2, IL-4, IL-7, IL-12, -interferon, GMCSP, BCG, aluminum hydroxide, MDP compounds, such as thur-MDP and nor-MDP, CGP (MTP-PE), lipid A, and monophosphoryl lipid A (MPL). Exemplary adjuvants may include complete Freund's adjuvant (a non-specific stimulator of the immune response containing killed Mycobacterium tuberculosis), incomplete Freund's adjuvants, and/or aluminum hydroxide adjuvant. In addition to adjuvants, it may be desirable to co-administer biologic response modifiers (BRM), such as but not limited to, Cimetidine (CIM; 1200 mg/d) (Smith/Kline, PA); low-dose Cyclophosphamide (CYP; 300 mg/m2) (Johnson/Mead, NJ), cytokines such as β-interferon, IL-2, or IL-12, or genes encoding proteins involved in immune helper functions, such as B-7.A phage-display system can be used to expand antibody molecule populations in vitro. Saiki, et al., Nature 324:163 (1986); Scharf et al., Science 233:1076 (1986); U.S. Pat. Nos. 4,683,195 and 4,683,202; Yang et al., J Mol Biol. 254:392 (1995); Barbas, III et al., Methods: Comp. Meth Enzymol. (1995) 8:94; Barbas, III et al., Proc Natl Acad Sci USA 88:7978 (1991).

### B. Fully Human Antibody Production

Methods are available for making fully human antibodies. Using fully human antibodies can minimize the immunogenic and allergic responses that may be caused by administering non-human monoclonal antibodies to humans as therapeutic agents. In one embodiment, human antibodies may be produced in a non-human transgenic animal, e.g., a transgenic mouse capable of producing multiple isotypes of human antibodies to protein (e.g., IgG, IgA, and/or IgE) by undergoing V-D-J recombination and isotype switching. Accordingly, this aspect applies to antibodies, antibody fragments, and pharmaceutical compositions thereof, but also non-human transgenic animals, B-cells, host cells, and hybridomas that produce monoclonal antibodies. Applications of human antibodies include, but are not limited to, detect a cell expressing an anticipated protein, either in vivo or in vitro, pharmaceutical preparations containing the antibodies of the present invention, and methods of treating disorders by administering the antibodies.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, for example, Jakobovits et al., Proc. Natl. Acad. Sci. USA 90:2551-2555 (1993); Jakobovits et al., Nature 362:255-258 (1993); Bruggermann et al., Year in Immunol. 7:33 (1993). In one example, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then crossbred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, International Patent Application Publication Nos. WO 96/33735 and WO 94/02602, which are hereby incorporated by reference in their entirety. Additional methods relating to transgenic mice for making human antibodies are described in U.S. Pat. Nos. 5,545,807; 6,713,610; 6,673,986; 6,162,963; 6,300,129; 6,255,458; 5,877,397; 5,874,299 and 5,545,806; in International Patent Application Publication Nos. WO 91/10741 and WO 90/04036; and in European Patent Nos. EP 546073B1 and EP 546073A1, all of which are hereby incorporated by reference in their entirety for all purposes.

The transgenic mice described above, referred to herein as “HuMAb” mice, contain a human immunoglobulin gene minilocus that encodes unrearranged human heavy (μ and γ) and κ light chain immunoglobulin sequences, together with targeted mutations that inactivate the endogenous μ and κ chain loci (Lonberg et al., Nature 368:856-859 (1994)). Accordingly, the mice exhibit reduced expression of mouse IgM or κ chains and in response to immunization, the introduced human heavy and light chain transgenes undergo class switching and somatic mutation to generate high affinity human IgG κ monoclonal antibodies (Lonberg et al., supra; Lonberg and Huszar, Intern. Ref. Immunol. 13:65-93 (1995); Harding and Lonberg, Ann. N.Y. Acad. Sci. 764:536-546 (1995)). The preparation of HuMAb mice is described in detail in Taylor et al., Nucl. Acids Res. 20:6287-6295 (1992); Chen et al., Int. Immunol. 5:647-656 (1993); Tuaillon et al., J. Immunol. 152:2912-2920 (1994); Lonberg et al., supra; Lonberg, Handbook of Exp. Pharmacol. 113:49-101 (1994); Taylor et al., Int. Immunol. 6:579-591 (1994); Lonberg and Huszar, Intern. Ref. Immunol. 13:65-93 (1995); Harding and Lonberg, Ann. N.Y. Acad. Sci. 764:536-546 (1995); Fishwild et al., Nat. Biotechnol. 14:845-851 (1996); the foregoing references are herein incorporated by reference in their entirety for all purposes. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807; as well as International Patent Application Publication Nos. WO 93/1227; WO 92/22646; and WO 92/03918, the disclosures of all of which are hereby incorporated by reference in their entirety for all purposes. Technologies utilized for producing human antibodies in these transgenic mice are disclosed also in WO 98/24893, and Mendez et al., Nat. Genetics 15:146-156 (1997), which are herein incorporated by reference. For example, the HCo7 and HCo12 transgenic mice strains can be used to generate human antibodies.

Using hybridoma technology, antigen-specific humanized monoclonal antibodies with the desired specificity can be produced and selected from the transgenic mice such as those described above. Such antibodies may be cloned and expressed using a suitable vector and host cell, or the antibodies can be harvested from cultured hybridoma cells. Fully human antibodies can also be derived from phage-display libraries (as disclosed in Hoogenboom et al., J. Mol. Biol. 227:381 (1991); and Marks et al., J. Mol. Biol. 222:581 (1991)). One such technique is described in International Patent Application Publication No. WO 99/10494 (herein incorporated by reference), which describes the isolation of high affinity and functional agonistic antibodies for MPL- and msk-receptors using such an approach.

### C. Antibody Fragments Production

Antibody fragments that retain the ability to recognize the antigen of interest will also find use herein. A number of antibody fragments are known in the art that comprise antigen-binding sites capable of exhibiting immunological binding properties of an intact antibody molecule and can be subsequently modified by methods known in the arts. Functional fragments, including only the variable regions of the heavy and light chains, can also be produced using standard techniques such as recombinant production or preferential proteolytic cleavage of immunoglobulin molecules. These fragments are known as Fv. See, e.g., Inbar et al., Proc. Nat. Acad. Sci. USA 69:2659-2662 (1972); Hochman et al., Biochem. 15:2706-2710 (1976); and Ehrlich et al., Biochem. 19:4091-4096 (1980).

Single-chain variable fragments (scFvs) may be prepared by fusing DNA encoding a peptide linker between DNAs encoding the two variable domain polypeptides (VL and VH). scFvs can form antigen-binding monomers, or they can form multimers (e.g., dimers, trimers, or tetramers), depending on the length of a flexible linker between the two variable domains (Kortt et al., Prot. Eng. 10:423 (1997); Kort et al., Biomol. Eng. 18:95-108 (2001)). By combining different VL- and VH-comprising polypeptides, one can form multimeric scFvs that bind to different epitopes (Kriangkum et al., Biomol. Eng. 18:31-40 (2001)). Antigen-binding fragments are typically produced by recombinant DNA methods known to those skilled in the art. Although the two domains of the Fv fragment, VL and VH, are coded for by separate genes, they can be joined using recombinant methods by a synthetic linker that enables them to be made as a single chain polypeptide (known as single chain Fv (sFv or scFv); see e.g., Bird et al., Science 242:423-426 (1988); and Huston et al., Proc. Natl. Acad. Sci. USA 85:5879-5883 (1988). Design criteria include determining the appropriate length to span the distance between the C-terminus of one chain and the N-terminus of the other, wherein the linker is generally formed from small hydrophilic amino acid residues that do not tend to coil or form secondary structures. Suitable linkers generally comprise polypeptide chains of alternating sets of glycine and serine residues, and may include glutamic acid and lysine residues inserted to enhance solubility. Antigen-binding fragments are screened for utility in the same manner as intact antibodies. Such fragments include those obtained by amino-terminal and/or carboxy-terminal deletions, where the remaining amino acid sequence is substantially identical to the corresponding positions in the naturally occurring sequence deduced, for example, from a full-length cDNA sequence.

Antibodies may also be generated using peptide analogs of the epitopic determinants disclosed herein, which may consist of non-peptide compounds having properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al., J. Med. Chem. 30:1229 (1987). Liu et al. (2003) also describe “antibody like binding peptidomimetics” (ABiPs), which are peptides that act as pared-down antibodies and have certain advantages of longer serum half-life as well as less cumbersome synthesis methods. These analogs can be peptides, non-peptides or combinations of peptide and non-peptide regions. Fauchere, Adv. Drug Res. 15:29 (1986); Veber and Freidiner, TINS p. 392 (1985); and Evans et al., J. Med. Chem. 30:1229 (1987), which are incorporated herein by reference in their entirety for any purpose. Peptide mimetics that are structurally similar to therapeutically useful peptides may be used to produce a similar therapeutic or prophylactic effect. Such compounds are often developed with the aid of computerized molecular modeling. Generally, peptidomimetics of the invention are proteins that are structurally similar to an antibody displaying a desired biological activity, such as the ability to bind a protein, but have one or more peptide linkages optionally replaced by a linkage selected from: CH2NH—, —CH2S—, —CH2-CH2-, —CH═CH— (cis and trans), —COCH2-, —CH(OH)CH2-, and —CH2SO— by methods well known in the art. Systematic substitution of one or more amino acids of a consensus sequence with a D-amino acid of the same type (e.g., D-lysine in place of L-lysine) may be used in certain embodiments of the invention to generate more stable proteins. In addition, constrained peptides comprising a consensus sequence or a substantially identical consensus sequence variation may be generated by methods known in the art (Rizo and Gierasch, Ann. Rev. Biochem. 61:387 (1992), incorporated herein by reference), for example, by adding internal cysteine residues capable of forming intramolecular disulfide bridges which cyclize the peptide.

Once generated, a phage display library can be used to improve the immunological binding affinity of the Fab molecules using known techniques. See, e.g., Figini et al., J. Mol. Biol. 239:68 (1994). The coding sequences for the heavy and light chain portions of the Fab molecules selected from the phage display library can be isolated or synthesized and cloned into any suitable vector or replicon for expression. Any suitable expression system can be used.

### III. OBTAINING ENCODED ANTIBODIES

In some aspects, there are nucleic acid molecule encoding antibody polypeptides (e.g., heavy or light chain, variable domain only, or full-length). These may be generated by methods known in the art, e.g., isolated from B cells of mice that have been immunized and isolated, phage display, expressed in any suitable recombinant expression system and allowed to assemble to form antibody molecules.

### A. Expression

The nucleic acid molecules may be used to express large quantities of recombinant antibodies or to produce chimeric antibodies, single chain antibodies, immunoadhesins, diabodies, mutated antibodies, and other antibody derivatives. If the nucleic acid molecules are derived from a non-human, non-transgenic animal, the nucleic acid molecules may be used for antibody humanization.

### 1. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody fragments, and probes thereof. In addition to control sequences that govern transcription and translation, vectors and expression vectors may contain nucleic acid sequences that serve other functions as well.

To express the antibodies, or antigen-binding fragments thereof, DNAs encoding partial or full-length light and heavy chains are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### 2. Expression Systems

Numerous expression systems exist that comprise at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### 3. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### 4. Host Cells

In another aspect, contemplated are the use of host cells into which a recombinant expression vector has been introduced. Antibodies can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the antibody expression construct can be placed under control of a promoter that is linked to T-cell activation, such as one that is controlled by NFAT-1 or NF-κB, both of which are transcription factors that can be activated upon T-cell activation. Control of antibody expression allows T cells, such as tumor-targeting T cells, to sense their surroundings and perform real-time modulation of cytokine signaling, both in the T cells themselves and in surrounding endogenous immune cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### B. Isolation

The nucleic acid molecule encoding either or both of the entire heavy and light chains of an antibody or the variable regions thereof may be obtained from any source that produces antibodies. Methods of isolating mRNA encoding an antibody are well known in the art. See e.g., Sambrook et al., supra. The sequences of human heavy and light chain constant region genes are also known in the art. See, e.g., Kabat et al., 1991, supra. Nucleic acid molecules encoding the full-length heavy and/or light chains may then be expressed in a cell into which they have been introduced and the antibody isolated.

### IV. ANTIBODIES, ANTIGEN BINDING FRAGMENTS, AND POLYPEPTIDES

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity. The term polypeptide also includes an antibody fragment described herein as well as antibody domains, such as HCDR1 (Heavy chain complementarity determining region 1), HCDR2 (Heavy chain complementarity determining region 2), HCDR3 (Heavy chain complementarity determining region 3), LCDR1 (Light chain complementarity determining region 1), LCDR2 (Light chain complementarity determining region 2), LCDR3 (Light chain complementarity determining region 3), HFRW1 (Heavy chain framework region 1), HFRW2 (Heavy chain framework region 2), HFRW3 (Heavy chain framework region 3), HFRW4 (Heavy chain framework region 4), LFRW1 (Light chain framework region 1), LFRW2 (Light chain framework region 21), LFRW3 (Light chain framework region 3), LFRW4 (Light chain framework region 4), VH (Heavy chain variable region), VL (Light chain variable region), CH (Heavy chain constant region), or CL (Light chain constant region).

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of an antibody, antigen binding fragment, protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The antibody, antigen binding fragment, polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NO:1-53 or 56-62.

In some embodiments, the antibody, antigen binding fragment, protein, or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of SEQ ID NOs:1-53 or 56-62.

In some embodiments, the antibody, antigen binding fragment, or polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids of SEQ ID NOs:1-53 or 56-62.

In some embodiments, the antibody, antigen binding fragment, protein, or polypeptide may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of SEQ ID NOs:1-53 or 56-62 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOs:1-53 or 56-62.

In some aspects there is a nucleic acid molecule, antibody, antigen binding fragment, protein, or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOs:1-53 or 56-62 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOs:1-53 or 56-62.

In some embodiments, the amino acid at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120,121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 of the heavy chain, light chain, VH, VL, HCDR1, HCDR2, HCDR3, LCDR1, LCDR2, LCDR3, HFRW1, HFRW2, HFRW3, HFRW4, LFRW1, LFRW2, LFRW3, or LFRW4 identified in Table 1 and SEQ ID NOs:1-53 or 56-62 is substituted with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In some embodiments, a polypeptide (e.g., antibody, antibody fragment, Fab, etc.) of the disclosure comprises a CDR that is at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical (or any range derivable therein) in sequence to one of SEQ ID NOs:1-53 or 56-62. In some embodiments, a polypeptide comprises 1, 2, and/or 3 CDRs from one of SEQ ID NOs:1-53 or 56-62. The CDR may be one that has been determined by Kabat, IMGT, or Chothia. In further embodiments, a polypeptide may have CDRs that have 1, 2, and/or 3 amino acid changes (e.g., addition of 1 or 2 amino acids, deletions of 1 or 2 amino acids, substitution) with respect to these 1, 2, or 3 CDRs. In some aspects, a polypeptide comprises additionally or alternatively, an amino acid sequence that is at least 60, 65, 70, 75, 80, 85, 90, 95, 96, 97, 98, 99, or 100% identical or homologous to the amino acid sequence of the variable region that is not a CDR sequence, i.e., the variable region framework.

From amino to carboxy terminus the CDRs are CDR1, CDR2, and CDR3. In some embodiments, a polypeptide may have CDRs that have 1, 2, and/or 3 amino acid changes (e.g., addition of 1 or 2 amino acids, deletions of 1 or 2 amino acids, substitution) with respect to CDR1, CDR2, or CDR3. In some embodiments, the CDRs of SEQ ID NOs:1-53 or 56-62 may further comprise 1, 2, 3, 4, 5, or 6 additional amino acids at the amino or carboxy terminus of the CDR, The additional amino acids may be from the heavy and/or light chain framework regions of SEQ ID NOs:1-53 or 56-62, that are shown as immediately adjacent to the CDRs. Accordingly, embodiments relate to polypeptides comprising an HCDR1 (i.e., CDR-H1), HCDR2 (i.e., CDR-H2), HCDR3 (i.e., CDR-H3), LCDR1 (i.e., CDR-L1), LCDR2 (i.e., CDR-L2), and/or LCDR3 (i.e., CDR-L3) with at least or at most or exactly 1, 2, 3, 4, 5, 6 or 7 amino acids at the amino end of the CDR or at the carboxy end of the CDR, wherein the additional amino acids are the 1, 2, 3, 4, 5, 6, or 7 amino acids of Table 1 or SEQ ID NOs:1-53 or 56-62 that are shown as immediately adjacent to the CDRs. Other embodiments relate to antibodies comprising one or more CDRs, wherein the CDR is a fragment of Table 1 or SEQ ID NOs:1-53 or 56-62 and wherein the fragment lacks 1, 2, 3, 4, or 5 amino acids from the amino or carboxy end of the CDR. In some embodiments, the CDR may lack one, 2, 3, 4, 5, 6, or 7 amino acids from the carboxy end and may further comprise 1, 2, 3, 4, 5, 6, 7, or 8 amino acids from the framework region of the amino end of the CDR. In some embodiments, the CDR may lack one, 2, 3, 4, 5, 6, or 7 amino acids from the amino end and may further comprise 1, 2, 3, 4, 5, 6, 7, or 8 amino acids from the framework region of the carboxy end of the CDR. In further embodiments, an antibody may be alternatively or additionally humanized in regions outside the CDR(s) and/or variable region(s). In some aspects, a polypeptide comprises additionally or alternatively, an amino acid sequence that is at least 60, 65, 70, 75, 80, 85, 90, 95, 96, 97, 98, 99, or 100% identical or homologous to the amino acid sequence of the variable region that is not a CDR sequence, i.e., the variable region framework.

In other embodiments, a polypeptide or protein comprises 1, 2, 3, 4, 5, or 6 CDRs from either or both of the light and heavy variable regions of Table 1 or SEQ ID NOs:1-53 or 56-62, and 1, 2, 3, 4, 5, or 6 CDRs may have 1, 2, and/or 3 amino acid changes with respect to these CDRs. In some embodiments, parts or all of the antibody sequence outside the variable region have been humanized. A protein may comprise one or more polypeptides. In some aspects, a protein may contain one or two polypeptides similar to a heavy chain polypeptide and/or 1 or 2 polypeptides similar to a light chain polypeptide.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### A. NT5E (CD73)

Aspects of the present disclosure are related to compositions and methods for identifying, detecting, and targeting NT5E. NT5E (also known as “ecto-5′-nucleotidase,” “5′-nucleotidase,” and “CD73”) is a 70 kDa 5′-ectonucleotidase that is extracellularly tethered by a glycosylphosphatidyl (GPI) anchor. NT5E exists as a homodimer, ˜125 kDa, both in solution and on the cell surface. The dimer interface is formed through contacts in the C-terminal domain. NT5E is expressed in many cell types including endothelial, T-lymphocyte, macrophage, and dendritic cells. NT5E, together with CD39 (NTPDase-1), is an important regulator of the adenosinergic pathway. Cell death or stress triggers release of ATP, which activates pro-inflammatory and immuno-stimulatory responses. ATP is down-regulated by sequential hydrolysis to ADP then to AMP, catalyzed by CD39. NT5E dephosphorylates AMP to adenosine, which then stimulates anti-inflammatory immuno-suppression.

NT5E is encoded by the gene NT5E. An example mRNA sequence encoding for human NT5E is available as NCBI RefSeq NM_002526. An example protein sequence for human NT5E is available as NCBI RefSeq NP_002517.

### V. SEQUENCES

Certain example polypeptide, antibody, and antigen binding fragment sequences are shown below in the following tables.

TABLE 1
Antibody and Antigen Binding Domain Sequences

| | | | SEQ |
|---|---|---|---|
| | Descrip- | | ID |
| Clone | tion | Sequence | NO: |
| 1780 | HCDR1 | NFYSYSI | 1 |
| | HCDR2 | SISSSSGYTY | 2 |
| | HCDR3 | YSWWGWHMEGWWGYMNAF | 3 |
| | LCDR1 | SVSSA | 4 |
| | LCDR2 | SASSLYS | 5 |
| | LCDR3 | AHPKPI | 6 |
| | VH | EVQLVESGGGLVQPGGSLRLSCAASGFNFYSYSIH | 7 |
| | | WVRQAPGKGLEWVASISSSSGYTYYADSVKGRFTI | |
| | | SADTSKNTAYLQMNSLRAEDTAVYYCARYSWWGWH | |
| | | MEGWWGYMNAFDYWGQGTLVTV | |
| | VL | SDIQMTQSPSSLSASVGDRVTITCRASQSVSSAVA | 8 |
| | | WYQQKPGKAPKLLIYSASSLYSGVPSRFSGSRSGT | |
| | | DFTLTISSLQPEDFATYYCQQAHPKPITFGQGTKV | |
| | | EIKRT | |
| | Full | EVQLVESGGGLVQPGGSLRLSCAASGENFYSYSIH | 9 |
| | heavy | WVRQAPGKGLEWVASISSSSGYTYYADSVKGRFTI | |
| | chain | SADTSKNTAYLQMNSLRAEDTAVYYCARYSWWGWH | |
| | | MEGWWGYMNAFDYWGQGTLVTVSSASTKGPSVFPL | |
| | | APSSKSTSGGTAALGCLVKDYFPEPVTVSWNSGAL | |
| | | TSGVHTFPAVLQSSGLYSLSSVVTVPSSSLGTQTY | |
| | | ICNVNHKPSNTKVDKKVEPKSCDKTHTCPPCPAPE | |
| | | LLGGPSVFLFPPKPKDTLMISRTPEVTCVVVDVSH | |
| | | EDPEVKFNWYVDGVEVHNAKTKPREEQYNSTYRVV | |
| | | SVLTVLHQDWLNGKEYKCKVSNKALPAPIEKTISK | |
| | | AKGQPREPQVYTLPPSREEMTKNQVSLTCLVKGFY | |
| | | PSDIAVEWESNGQPENNYKTTPPVLDSDGSFFLYS | |
| | | KLTVDKSRWQQGNVFSCSVMHEALHNHYTQKSLSL | |
| | | SPGK | |
| | Full | SDIQMTQSPSSLSASVGDRVTITCRASQSVSSAVA | 10 |
| | light | WYQQKPGKAPKLLIYSASSLYSGVPSRFSGSRSGT | |
| | chain | DFTLTISSLQPEDFATYYCQQAHPKPITFGQGTKV | |
| | | EIKRTVAAPSVFIFPPSDEQLKSGTASVVCLLNNF | |
| | | YPREAKVQWKVDNALQSGNSQESVTEQDSKDSTYS | |
| | | LSSTLTLSKADYEKHKVYACEVTHQGLSSPVTKSF | |
| | | NRGEC | |
| 1789 | HCDR1 | NVSYSSI | 11 |
| | HCDR2 | SISSSSGYTY | 2 |
| | HCDR3 | NYWGWYPFYYFAF | 13 |
| | LCDR1 | SVSSA | 4 |
| | LCDR2 | SASSLYS | 5 |
| | LCDR3 | GWDYPI | 16 |
| | VH | EVQLVESGGGLVQPGGSLRLSCAASGFNVSYSSIH | 17 |
| | | WVRQAPGKGLEWVASISSSSGYTYYADSVKGRFTI | |
| | | SADTSKNTAYLQMNSLRAEDTAVYYCARNYWGWYP | |
| | | FYYFAFDYWGQGTLVTV | |
| | VL | SDIQMTQSPSSLSASVGDRVTITCRASQSVSSAVA | 18 |
| | | WYQQKPGKAPKLLIYSASSLYSGVPSRFSGSRSGT | |
| | | DFTLTISSLQPEDFATYYCQQGWDYPITFGQGTKV | |
| | | EIKRT | |
| | Full | EVQLVESGGGLVQPGGSLRLSCAASGFNVSYSSIH | 19 |
| | heavy | WVRQAPGKGLEWVASISSSSGYTYYADSVKGRFTI | |
| | chain | SADTSKNTAYLQMNSLRAEDTAVYYCARNYWGWYP | |
| | | FYYFAFDYWGQGTLVTVSSASTKGPSVFPLAPSSK | |
| | | STSGGTAALGCLVKDYFPEPVTVSWNSGALTSGVH | |
| | | TFPAVLQSSGLYSLSSVVTVPSSSLGTQTYICNVN | |
| | | HKPSNTKVDKKVEPKSCDKTHTCPPCPAPELLGGP | |
| | | SVFLFPPKPKDTLMISRTPEVTCVVVDVSHEDPEV | |
| | | KFNWYVDGVEVHNAKTKPREEQYNSTYRVVSVLTV | |
| | | LHQDWLNGKEYKCKVSNKALPAPIEKTISKAKGQP | |
| | | REPQVYTLPPSREEMTKNQVSLTCLVKGFYPSDIA | |
| | | VEWESNGQPENNYKTTPPVLDSDGSFFLYSKLTVD | |
| | | KSRWQQGNVFSCSVMHEALHNHYTQKSLSLSPGK | |
| | Full | SDIQMTQSPSSLSASVGDRVTITCRASQSVSSAVA | 20 |
| | light | WYQQKPGKAPKLLIYSASSLYSGVPSRFSGSRSGT | |
| | chain | DFTLTISSLQPEDFATYYCQQGWDYPITFGQGTKV | |
| | | EIKRTVAAPSVFIFPPSDEQLKSGTASVVCLLNNF | |
| | | YPREAKVQWKVDNALQSGNSQESVTEQDSKDSTYS | |
| | | LSSTLTLSKADYEKHKVYACEVTHQGLSSPVTKSF | |
| | | NRGEC | |
| | HCDR1 | VSYSSI | 21 |
| 1789 | HCDR2 | RIRGNEGDTY | 22 |
| AM32 | HCDR3 | NYWGWYPFYYFAF | 13 |
| | LCDR1 | SVSSA | 4 |
| | LCDR2 | SASSLYS | 5 |
| | LCDR3 | GWDYPI | 16 |
| | VH | EVQLVESGGGLVQPGGSLRLSCAASGFNVSYSSIH | 27 |
| | | WVRQAPGKGLEWVARIRGNEGDTYYADSVKGRFTI | |
| | | SADTSKNTAYLQMNSLRAEDTAVYYCARNYWGWYP | |
| | | FYYFAFDYWGQGTLVTV | |
| | VL | SDIQMTQSPSSLSASVGDRVTITCRASQSVSSAVA | 18 |
| | | WYQQKPGKAPKLLIYSASSLYSGVPSRFSGSRSGT | |
| | | DFTLTISSLQPEDFATYYCQQGWDYPITFGQGTKV | |
| | | EIKRT | |
| | Full | EVQLVESGGGLVQPGGSLRLSCAASGFNVSYSSIH | 29 |
| | heavy | WVRQAPGKGLEWVARIRGNEGDTYYADSVKGRFTI | |
| | chain | SADTSKNTAYLQMNSLRAEDTAVYYCARNYWGWYP | |
| | | FYYFAFDYWGQGTLVTVSSASTKGPSVFPLAPSSK | |
| | | STSGGTAALGCLVKDYFPEPVTVSWNSGALTSGVH | |
| | | TFPAVLQSSGLYSLSSVVTVPSSSLGTQTYICNVN | |
| | | HKPSNTKVDKKVEPKSCDKTHTCPPCPAPELLGGP | |
| | | SVFLFPPKPKDTLMISRTPEVTCVVVDVSHEDPEV | |
| | | KFNWYVDGVEVHNAKTKPREEQYNSTYRVVSVLTV | |
| | | LHQDWLNGKEYKCKVSNKALPAPIEKTISKAKGQP | |
| | | REPQVYTLPPSREEMTKNQVSLTCLVKGFYPSDIA | |
| | | VEWESNGQPENNYKTTPPVLDSDGSFFLYSKLTVD | |
| | | KSRWQQGNVFSCSVMHEALHNHYTQKSLSLSPGK | |
| | Full | SDIQMTQSPSSLSASVGDRVTITCRASQSVSSAVA | 30 |
| | light | WYQQKPGKAPKLLIYSASSLYSGVPSRFSGSRSGT | |
| | chain | DFTLTISSLQPEDFATYYCQQGWDYPITFGQGTKV | |
| | | EIKRTVAAPSVFIFPPSDSQLKSGTASVVCLLNNF | |
| | | YPREAKVQWKVDNALQSGNSQESVTEQDSKDSTYS | |
| | | LSSTLTLSKADYEKHKVYACEVTHQGLSSPVTKSF | |
| | | NRGEC | |
| 1789 | HCDR1 | VSYSSI | 21 |
| AM39 | | | |
| | HCDR2 | SIRGAGSDTR | 32 |
| | HCDR3 | NYWGWYPFYYFAF | 13 |
| | LCDR1 | SVSSA | 4 |
| | LCDR2 | SASSLYS | 5 |
| | LCDR3 | GWDYPI | 16 |
| | VH | EVQLVESGGGLVQPGGSLRLSCAASGFNVSYSSIH | 37 |
| | | WVRQAPGKGLEWVASIRGAGSDTRYADSVKGRFTI | |
| | | SADTSKNTAYLQMNSLRAEDTAVYYCARNYWGWYP | |
| | | FYYFAFDYWGQGTLVTV | |
| | VL | SDIQMTQSPSSLSASVGDRVTITCRASQSVSSAVA | 18 |
| | | WYQQKPGKAPKLLIYSASSLYSGVPSRFSGSRSGT | |
| | | DFTLTISSLQPEDFATYYCQQGWDYPITFGQGTKV | |
| | | EIKRT | |
| | Full | EVQLVESGGGLVQPGGSLRLSCAASGFNVSYSSIH | 39 |
| | heavy | WVRQAPGKGLEWVASIRGAGSDTRYADSVKGRFTI | |
| | chain | SADTSKNTAYLQMNSLRAEDTAVYYCARNYWGWYP | |
| | | FYYFAFDYWGQGTLVTVSSASTKGPSVFPLAPSSK | |
| | | STSGGTAALGCLVKDYFPEPVTVSWNSGALTSGVH | |
| | | TFPAVLQSSGLYSLSSVVTVPSSSLGTQTYICNVN | |
| | | HKPSNTKVDKKVEPKSCDKTHTCPPCPAPELLGGP | |
| | | SVFLFPPKPKDTLMISRTPEVTCVVVDVSHEDPEV | |
| | | KFNWYVDGVEVHNAKTKPREEQYNSTYRVVSVLTV | |
| | | LHQDWLNGKEYKCKVSNKALPAPIEKTISKAKGQP | |
| | | REPQVYTLPPSREEMTKNQVSLTCLVKGFYPSDIA | |
| | | VEWESNGQPENNYKTTPPVLDSDGSFFLYSKLTVD | |
| | | KSRWQQGNVFSCSVMHEALHNHYTQKSLSLSPGK | |
| | Full | SDIQMTQSPSSLSASVGDRVTITCRASQSVSSAVA | 30 |
| | light | WYQQKPGKAPKLLIYSASSLYSGVPSRFSGSRSGT | |
| | chain | DFTLTISSLQPEDFATYYCQQGWDYPITFGQGTKV | |
| | | EIKRTVAAPSVFIFPPSDSQLKSGTASVVCLLNNF | |
| | | YPREAKVQWKVDNALQSGNSQESVTEQDSKDSTYS | |
| | | LSSTLTLSKADYEKHKVYACEVTHQGLSSPVTKSF | |
| | | NRGEC | |
| 1789 | HCDR1 | VSYSSI | 21 |
| AM42 | | | |
| | HCDR2 | KIRGGHQDTH | 42 |
| | HCDR3 | NYWGWYPFYYFAF | 13 |
| | LCDR1 | SVSSA | 4 |
| | LCDR2 | SASSLYS | 5 |
| | LCDR3 | GWDYPI | 16 |
| | VH | EVQLVESGGGLVQPGGSLRLSCAASGFNVSYSSIH | 47 |
| | | WVRQAPGKGLEWVAKIRGGHQDTHYADSVKGRFTI | |
| | | SADTSKNTAYLQMNSLRAEDTAVYYCARNYWGWYP | |
| | | FYYFAFDYWGQGTLVTV | |
| | VL | SDIQMTQSPSSLSASVGDRVTITCRASQSVSSAVA | 18 |
| | | WYQQKPGKAPKLLIYSASSLYSGVPSRFSGSRSGT | |
| | | DFTLTISSLQPEDFATYYCQQGWDYPITFGQGTKV | |
| | | EIKRT | |
| | Full | EVQLVESGGGLVQPGGSLRLSCAASGFNVSYSSIH | 49 |
| | heavy | WVRQAPGKGLEWVAKIRGGHQDTHYADSVKGRFTI | |
| | chain | SADTSKNTAYLQMNSLRAEDTAVYYCARNYWGWYP | |
| | | FYYFAFDYWGQGTLVTVSSASTKGPSVFPLAPSSK | |
| | | STSGGTAALGCLVKDYFPEPVTVSWNSGALTSGVH | |
| | | TFPAVLQSSGLYSLSSVVTVPSSSLGTQTYICNVN | |
| | | HKPSNTKVDKKVEPKSCDKTHTCPPCPAPELLGGP | |
| | | SVFLFPPKPKDTLMISRTPEVTCVVVDVSHEDPEV | |
| | | KFNWYVDGVEVHNAKTKPREEQYNSTYRVVSVLTV | |
| | | LHQDWLNGKEYKCKVSNKALPAPIEKTISKAKGQP | |
| | | REPQVYTLPPSREEMTKNQVSLTCLVKGFYPSDIA | |
| | | VEWESNGQPENNYKTTPPVLDSDGSFFLYSKLTVD | |
| | | KSRWQQGNVFSCSVMHEALHNHYTQKSLSLSPGK | |
| | Full | SDIQMTQSPSSLSASVGDRVTITCRASQSVSSAVA | 30 |
| | light | WYQQKPGKAPKLLIYSASSLYSGVPSRFSGSRSGT | |
| | chain | DFTLTISSLQPEDFATYYCQQGWDYPITFGQGTKV | |
| | | EIKRTVAAPSVFIFPPSDSQLKSGTASVVCLLNNF | |
| | | YPREAKVQWKVDNALQSGNSQESVTEQDSKDSTYS | |
| | | LSSTLTLSKADYEKHKVYACEVTHQGLSSPVTKSF | |
| | | NRGEC | |
| 1798 | HCDR1 | FSYSSI | 51 |
| | HCDR2 | SISSSYGSTY | 52 |
| | HCDR3 | AFYSHDYRYIYWGSGM | 53 |
| | LCDR1 | SVSSA | 4 |
| | LCDR2 | SASSLYS | 5 |
| | LCDR3 | SWYYPF | 56 |
| | VH | EVQLVESGGGLVQPGGSLRLSCAASGFNFSYSSIH | 57 |
| | | WVRQAPGKGLEWVASISSSYGSTYYADSVKGRFTI | |
| | | SADTSKNTAYLQMNSLRAEDTAVYYCARAFYSHDY | |
| | | RYIYWGSGMDYWGQGTLVTV | |
| | VL | SDIQMTQSPSSLSASVGDRVTITCRASQSVSSAVA | 58 |
| | | WYQQKPGKAPKLLIYSASSLYSGVPSRFSGSRSGT | |
| | | DFTLTISSLQPEDFATYYCQQSWYYPFTFGQGTKV | |
| | | EIKRT | |
| | Full | EVQLVESGGGLVQPGGSLRLSCAASGFNFSYSSIH | 59 |
| | heavy | WVRQAPGKGLEWVASISSSYGSTYYADSVKGRFTI | |
| | chain | SADTSKNTAYLQMNSLRAEDTAVYYCARAFYSHDY | |
| | | RYIYWGSGMDYWGQGTLVTVSSASTKGPSVFPLAP | |
| | | SSKSTSGGTAALGCLVKDYFPEPVTVSWNSGALTS | |
| | | GVHTFPAVLQSSGLYSLSSVVTVPSSSLGTQTYIC | |
| | | NVNHKPSNTKVDKKVEPKSCDKTHTCPPCPAPELL | |
| | | GGPSVFLFPPKPKDTLMISRTPEVTCVVVDVSHED | |
| | | PEVKFNWYVDGVEVHNAKTKPREEQYNSTYRVVSV | |
| | | LTVLHQDWLNGKEYKCKVSNKALPAPIEKTISKAK | |
| | | GQPREPQVYTLPPSREEMTKNQVSLTCLVKGFYPS | |
| | | DIAVEWESNGQPENNYKTTPPVLDSDGSFFLYSKL | |
| | | TVDKSRWQQGNVFSCSVMHEALHNHYTQKSLSLSP | |
| | | GK | |
| | Full | SDIQMTQSPSSLSASVGDRVTITCRASQSVSSAVA | 60 |
| | light | WYQQKPGKAPKLLIYSASSLYSGVPSRFSGSRSGT | |
| | chain | DFTLTISSLQPEDFATYYCQQSWYYPFTFGQGTKV | |
| | | EIKRTVAAPSVFIFPPSDSQLKSGTASVVCLLNNF | |
| | | YPREAKVQWKVDNALQSGNSQESVTEQDSKDSTYS | |
| | | LSSTLTLSKADYEKHKVYACEVTHQGLSSPVTKSF | |
| | | NRGEC | |
| 1557 | HCDR1 | VYYSSI | 61 |
| | HCDR2 | SISSSSGSTS | 62 |
| | HCDR3 | YGWWEYGYGGWWHPAL | 12 |
| | LCDR1 | SVSSA | 4 |
| | LCDR2 | SASSLYS | 5 |
| | LCDR3 | VWWGPI | 14 |
| | VH | EVQLVESGGGLVQPGGSLRLSCAASGENVYYSSIH | 15 |
| | | WVRQAPGKGLEWVASISSSSGSTSYADSVKGRFTI | |
| | | SADTSKNTAYLQMNSLRAEDTAVYYCARYGWWEYG | |
| | | YGGWWHPALDYWGQGTLVTV | |
| | VL | SDIQMTQSPSSLSASVGDRVTITCRASQSVSSAVA | 23 |
| | | WYQQKPGKAPKLLIYSASSLYSGVPSRFSGSRSGT | |
| | | DFTLTISSLQPEDFATYYCQQVWWGPITFGQGTKV | |
| | | EIKRT | |
| | Full | EVQLVESGGGLVQPGGSLRLSCAASGENVYYSSIH | 24 |
| | heavy | WVRQAPGKGLEWVASISSSSGSTSYADSVKGRFTI | |
| | chain | SADTSKNTAYLQMNSLRAEDTAVYYCARYGWWEYG | |
| | | YGGWWHPALDYWGQGTLVTVSSASTKGPSVFPLAP | |
| | | SSKSTSGGTAALGCLVKDYFPEPVTVSWNSGALTS | |
| | | GVHTFPAVLQSSGLYSLSSVVTVPSSSLGTQTYIC | |
| | | NVNHKPSNTKVDKKVEPKSCDKTHTCPPCPAPELL | |
| | | GGPSVFLFPPKPKDTLMISRTPEVTCVVVDVSHED | |
| | | PEVKFNWYVDGVEVHNAKTKPREEQYNSTYRVVSV | |
| | | LTVLHQDWLNGKEYKCKVSNKALPAPIEKTISKAK | |
| | | GQPREPQVYTLPPSREEMTKNQVSLTCLVKGFYPS | |
| | | DIAVEWESNGQPENNYKTTPPVLDSDGSFFLYSKL | |
| | | TVDKSRWQQGNVFSCSVMHEALHNHYTQKSLSLSP | |
| | | GK | |
| | Full | SDIQMTQSPSSLSASVGDRVTITCRASQSVSSAVA | 25 |
| | light | WYQQKPGKAPKLLIYSASSLYSGVPSRFSGSRSGT | |
| | chain | DFTLTISSLQPEDFATYYCQQVWWGPITFGQGTKV | |
| | | EIKRTVAAPSVFIFPPSDSQLKSGTASVVCLLNNF | |
| | | YPREAKVQWKVDNALQSGNSQESVTEQDSKDSTYS | |
| | | LSSTLTLSKADYEKHKVYACEVTHQGLSSPVTKSF | |
| | | NRGEC | |

TABLE 1.1
NT5E Sequences

| | | | SEQ |
|---|---|---|---|
| | Descrip- | | ID |
| Clone | tion | Sequence | NO: |
| NT5E | Full- | MLLVNQSHQGFNKEHTSKMVSAIVLYVLLA | 65 |
| | length | AAAHSAFAADPHHHHHHGWELTILHTNDVH | |
| | human | SRLEQTSEDSSKCVNASRCMGGVARLFTKV | |
| | NT5E | QQIRRAEPNVLLLDAGDQYQGTIWFTVYKG | |
| | | AEVAHFMNALRYDAMALGNHEFDNGVEGLI | |
| | | EPLLKEAKFPILSANIKAKGPLASQISGLY | |
| | | LPYKVLPVGDEVVGIVGYTSKETPFLSNPG | |
| | | TNLVFEDEITALQPEVDKLKTLNVNKIIAL | |
| | | GHSGFEMDKLIAQKVRGVDVVVGGHSNTFL | |
| | | YTGNPPSKEVPAGKYPFIVTSDDGRKVPVV | |
| | | QAYAFGKYLGYLKIEFDERGNVISSHGNPI | |
| | | LLNSSIPEDPSIKADINKWRIKLDNYSTQE | |
| | | LGKTIVYLDGSSQSCRFRECNMGNLICDAM | |
| | | INNNLRHADETFWNHVSMCILNGGGIRSPI | |
| | | DERNNGTITWENLAAVLPFGGTFDLVQLKG | |
| | | STLKKAFEHSVHRYGQSTGEFLQVGGIHVV | |
| | | YDLSRKPGDRVVKLDVLCTKCRVPSYDPLK | |
| | | MDEVYKVILPNFLANGGDGFQMIKDELLRH | |
| | | DSGDQDINVVSTYISKMKVIYPAVEGRIKF | |
| | | SATGASGGGGGSGLNDIFEAQKIEWHEG | |
| | Full- | MLLVNQSHQGFNKEHTSKMVSAIVLYVLLA | 54 |
| | length | AAAHSAFAADPHHHHHHGWELTILHTNDVH | |
| | murine | SRLEQTSDDSTKCLNASLCVGGVARLFTKV | |
| | NT5E | QQIRKEEPNVLFLDAGDQYQGTIWFTVYKG | |
| | | LEVAHFMNILGYDAMALGNHEFDNGVEGLI | |
| | | DPLLRNVKFPILSANIKARGPLAHQISGLF | |
| | | LPSKVLSVGGEVVGIVGYTSKETPFLSNPG | |
| | | TNLVFEDEISALQPEVDKLKTLNVNKIIAL | |
| | | GHSGFEMDKLIAQKVRGVDIVVGGHSNTFL | |
| | | YTGNPPSKEVPAGKYPFIVTADDGRQVPVV | |
| | | QAYAFGKYLGYLKVEFDDKGNVITSYGNPI | |
| | | LLNSSIPEDATIKADINQWRIKLDNYSTQE | |
| | | LGRTIVYLDGSTQTCRFRECNMGNLICDAM | |
| | | INNNLRHPDEMFWNHVSMCIVNGGGIRSPI | |
| | | DEKNNGTITWENLAAVLPFGGTFDLVQLKG | |
| | | STLKKAFEHSVHRYGQSTGEFLQVGGIHVV | |
| | | YDINRKPWNRVVQLEVLCTKCRVPIYEPLE | |
| | | MDKVYKVTLPSYLANGGDGFQMIKDELLKH | |
| | | DSGDQDISVVSEYISKMKVVYPAVEGRIKF | |
| | | SATGASGGGGGSGLNDIFEAQKIEWHEG | |
| | Full- | MLLVNQSHQGFNKEHTSKMVSAIVLYVLLA | 55 |
| | length | AAAHSAFAADPHHHHHHGWELTILHTNDVH | |
| | cyno- | SRLEQTSEDSSKCVNASRCMGGVARLFTKV | |
| | molgus | QQIRRAEPNVLLLDAGDQYQGTIWFTVYKG | |
| | NT5E | AEVAHFMNALRYDAMALGNHEFDNGVEGLI | |
| | | EPLLKEAKFPILSANIKAKGPLASQISGLY | |
| | | LPYKVLPVGDEVVGIVGYTSKETPFLSNPG | |
| | | TNLVFEDEITALQPEVDKLKTLNVNKIIAL | |
| | | GHSGFETDKLIAQKVRGVDVVVGGHSNTFL | |
| | | YTGNPPSKEVPAGKYPFIVTSDDGRKVPVV | |
| | | QAYAFGKYLGYLKIEFDERGNVISSHGNPI | |
| | | LLNSSIPEDPSIKADINKWRIKLDNYSTQE | |
| | | LGKTIVYLDGSSQSCRFRECNMGNLICDAM | |
| | | INNNLRHADEMFWNHVSMCILNGGGIRSPI | |
| | | DERNNGTITWENLAAVLPFGGTFDLVQLKG | |
| | | STLKKAFEHSVHRYGQSTGEFLQVGGIHVV | |
| | | YDLSRKPGDRVVKLDVLCTKCRVPSYDPLK | |
| | | MDEIYKVILPNFLANGGDGFQMIKDELLRH | |
| | | DSGDQDINVVSTYISKMKVIYPAVEGRIKF | |
| | | SATGASGGGGGSGLNDIFEAQKIEWHEG | |
| | N-term- | MLLVNQSHQGFNKEHTSKMVSAIVLYVLLA | 63 |
| | inal | AAAHSAFAADPWELTILHTNDVHSRLEQTS | |
| | domain | EDSSKCVNASRCMGGVARLFTKVQQIRRAE | |
| | of | PNVLLLDAGDQYQGTIWFTVYKGAEVAHFM | |
| | human | NALRYDAMALGNHEFDNGVEGLIEPLLKEA | |
| | NT5E | KFPILSANIKAKGPLASQISGLYLPYKVLP | |
| | | VGDEVVGIVGYTSKETPFLSNPGTNLVFED | |
| | | EITALQPEVDKLKTLNVNKIIALGHSGFEM | |
| | | DKLIAQKVRGVDVVVGGHSNTFLYTGNPPS | |
| | | KEVPAGKYPFIVTSDDGRKVPVVQAYAFGK | |
| | | YLGYLKIEFDERGNVISSHGNPILLNSSIP | |
| | | EDPSIKADINKWRIKLDNYSHHHHHH | |
| | C-term- | MLLVNQSHQGFNKEHTSKMVSAIVLYVLLA | 64 |
| | inal | AAAHSAFAADPHHHHHHELGKTIVYLDGSS | |
| | domain | QSCRFRECNMGNLICDAMINNNLRHADETF | |
| | of | WNHVSMCILNGGGIRSPIDERNNGTITWEN | |
| | human | LAAVLPFGGTFDLVQLKGSTLKKAFEHSVH | |
| | NT5E | RYGQSTGEFLQVGGIHVVYDLSRKPGDRVV | |
| | | KLDVLCTKCRVPSYDPLKMDEVYKVILPNF | |
| | | LANGGDGFQMIKDELLRHDSGDQDINVVST | |
| | | YISKMKVIYPAVEGRIKFSATGASGGGGGS | |
| | | GLNDIFEAQKIEWHEG | |

TABLE 2
Nucleic Acid Sequences

| | | | SEQ ID |
|---|---|---|---|
| Clone | Description | Sequence | NO: |
| 1780 | Heavy | GAGGTTCAGCTGGTGGAGTCTGGCGGTGGCCTGGTG | 26 |
| | Chain | CAGCCAGGGGGCTCACTCCGTTTGTCCTGTGCAGCT | |
| | | TCTGGCTTCAACTTCTATTCTTATTCTATACACTGG | |
| | | GTGCGTCAGGCCCCGGGTAAGGGCCTGGAATGGGTT | |
| | | GCATCTATTTCTTCTTCTTCTGGCTATACTTATTAT | |
| | | GCCGATAGCGTCAAGGGCCGTTTCACTATAAGCGCA | |
| | | GACACATCCAAAAACACAGCCTACCTACAAATGAAC | |
| | | AGCTTAAGAGCTGAGGACACTGCCGTCTATTATTGT | |
| | | GCTCGCTACTCTTGGTGGGGTTGGCATATGGAAGGT | |
| | | TGGTGGGGTTACATGAACGCTTTTGACTACTGGGGT | |
| | | CAAGGAACCCTGGTCACCGTCTCCTCGGCTAGCACC | |
| | | AAGGGCCCATCGGTCTTCCCCCTGGCACCCTCCTCC | |
| | | AAGAGCACCTCTGGGGGCACAGCGGCCCTGGGCTGC | |
| | | CTGGTCAAGGACTACTTCCCCGAACCGGTGACGGTG | |
| | | TCGTGGAACTCAGGCGCCCTGACCAGCGGCGTGCAC | |
| | | ACCTTCCCGGCTGTCCTACAGTCCTCAGGACTCTAC | |
| | | TCCCTCAGCAGCGTGGTGACCGTGCCCTCCAGCAGC | |
| | | TTGGGCACCCAGACCTACATCTGCAACGTGAATCAC | |
| | | AAGCCCAGCAACACCAAGGTGGACAAGAAAGTTGAG | |
| | | CCCAAATCTTGTGACAAAACTCACACATGCCCACCG | |
| | | TGCCCAGCACCTGAACTCCTGGGGGGACCGTCAGTC | |
| | | TTCCTCTTCCCCCCAAAACCCAAGGACACCCTCATG | |
| | | ATCTCCCGGACCCCTGAGGTCACATGCGTGGTGGTG | |
| | | GACGTGAGCCACGAAGACCCTGAGGTCAAGTTCAAC | |
| | | TGGTACGTGGACGGCGTGGAGGTGCATAATGCCAAG | |
| | | ACAAAGCCGCGGGAGGAGCAGTACAACAGCACGTAC | |
| | | CGTGTGGTCAGCGTCCTCACCGTCCTGCACCAGGAC | |
| | | TGGCTGAATGGCAAGGAGTACAAGTGCAAGGTCTCC | |
| | | AACAAAGCCCTCCCAGCCCCCATCGAGAAAACCATC | |
| | | TCCAAAGCCAAAGGGCAGCCCCGAGAACCACAGGTG | |
| | | TACACCCTGCCCCCATCCCGGGAGGAGATGACCAAG | |
| | | AACCAGGTCAGCCTGACCTGCCTGGTCAAAGGCTTC | |
| | | TATCCCAGCGACATCGCCGTGGAGTGGGAGAGCAAT | |
| | | GGGCAGCCGGAGAACAACTACAAGACCACGCCTCCC | |
| | | GTGCTGGACTCCGACGGCTCCTTCTTCCTCTACAGC | |
| | | AAGCTCACCGTGGACAAGAGCAGGTGGCAGCAGGGG | |
| | | AACGTCTTCTCATGCTCCGTGATGCATGAGGCTCTG | |
| | | CACAACCACTACACGCAGAAGAGCCTCTCCCTGTCT | |
| | | CCGGGTAAA | |
| 1789 | Heavy | GAGGTTCAGCTGGTGGAGTCTGGCGGTGGCCTGGTG | 28 |
| | Chain | CAGCCAGGGGGCTCACTCCGTTTGTCCTGTGCAGCT | |
| | | TCTGGCTTCAACGTCTCTTATTCTTCTATACACTGG | |
| | | GTGCGTCAGGCCCCGGGTAAGGGCCTGGAATGGGTT | |
| | | GCATCTATTTCTTCTTCTTCTGGCTATACTTATTAT | |
| | | GCCGATAGCGTCAAGGGCCGTTTCACTATAAGCGCA | |
| | | GACACATCCAAAAACACAGCCTACCTACAAATGAAC | |
| | | AGCTTAAGAGCTGAGGACACTGCCGTCTATTATTGT | |
| | | GCTCGCAACTACTGGGGTTGGTACCCGTTCTACTAC | |
| | | TTCGCTTTTGACTACTGGGGTCAAGGAACCCTGGTC | |
| | | ACCGTCTCCTCGGCTAGCACCAAGGGCCCATCGGTC | |
| | | TTCCCCCTGGCACCCTCCTCCAAGAGCACCTCTGGG | |
| | | GGCACAGCGGCCCTGGGCTGCCTGGTCAAGGACTAC | |
| | | TTCCCCGAACCGGTGACGGTGTCGTGGAACTCAGGC | |
| | | GCCCTGACCAGCGGCGTGCACACCTTCCCGGCTGTC | |
| | | CTACAGTCCTCAGGACTCTACTCCCTCAGCAGCGTG | |
| | | GTGACCGTGCCCTCCAGCAGCTTGGGCACCCAGACC | |
| | | TACATCTGCAACGTGAATCACAAGCCCAGCAACACC | |
| | | AAGGTGGACAAGAAAGTTGAGCCCAAATCTTGTGAC | |
| | | AAAACTCACACATGCCCACCGTGCCCAGCACCTGAA | |
| | | CTCCTGGGGGGACCGTCAGTCTTCCTCTTCCCCCCA | |
| | | AAACCCAAGGACACCCTCATGATCTCCCGGACCCCT | |
| | | GAGGTCACATGCGTGGTGGTGGACGTGAGCCACGAA | |
| | | GACCCTGAGGTCAAGTTCAACTGGTACGTGGACGGC | |
| | | GTGGAGGTGCATAATGCCAAGACAAAGCCGCGGGAG | |
| | | GAGCAGTACAACAGCACGTACCGTGTGGTCAGCGTC | |
| | | CTCACCGTCCTGCACCAGGACTGGCTGAATGGCAAG | |
| | | GAGTACAAGTGCAAGGTCTCCAACAAAGCCCTCCCA | |
| | | GCCCCCATCGAGAAAACCATCTCCAAAGCCAAAGGG | |
| | | CAGCCCCGAGAACCACAGGTGTACACCCTGCCCCCA | |
| | | TCCCGGGAGGAGATGACCAAGAACCAGGTCAGCCTG | |
| | | ACCTGCCTGGTCAAAGGCTTCTATCCCAGCGACATC | |
| | | GCCGTGGAGTGGGAGAGCAATGGGCAGCCGGAGAAC | |
| | | AACTACAAGACCACGCCTCCCGTGCTGGACTCCGAC | |
| | | GGCTCCTTCTTCCTCTACAGCAAGCTCACCGTGGAC | |
| | | AAGAGCAGGTGGCAGCAGGGGAACGTCTTCTCATGC | |
| | | TCCGTGATGCATGAGGCTCTGCACAACCACTACACG | |
| | | CAGAAGAGCCTCTCCCTGTCTCCGGGTAAA | |
| 1789 | Heavy | GAGGTTCAGCTGGTGGAGTCTGGCGGTGGCCTGGTG | 31 |
| AM32 | Chain | CAGCCAGGGGGCTCACTCCGTTTGTCCTGTGCAGCT | |
| | | TCTGGCTTCAACGTCTCTTATTCTTCTATACACTGG | |
| | | GTGCGTCAGGCCCCGGGTAAGGGCCTGGAATGGGTT | |
| | | GCACGTATTAGAGGAAACGAAGGGGACACTTACTAT | |
| | | GCCGATAGCGTCAAGGGCCGTTTCACTATAAGCGCA | |
| | | GACACATCCAAAAACACAGCCTACCTACAAATGAAC | |
| | | AGCTTAAGAGCTGAGGACACTGCCGTCTATTATTGT | |
| | | GCTCGCAACTACTGGGGTTGGTACCCGTTCTACTAC | |
| | | TTCGCTTTTGACTACTGGGGTCAAGGAACCCTGGTC | |
| | | ACCGTCTCCTCGGCTAGCACCAAGGGCCCATCGGTC | |
| | | TTCCCCCTGGCACCCTCCTCCAAGAGCACCTCTGGG | |
| | | GGCACAGCGGCCCTGGGCTGCCTGGTCAAGGACTAC | |
| | | TTCCCCGAACCGGTGACGGTGTCGTGGAACTCAGGC | |
| | | GCCCTGACCAGCGGCGTGCACACCTTCCCGGCTGTC | |
| | | CTACAGTCCTCAGGACTCTACTCCCTCAGCAGCGTG | |
| | | GTGACCGTGCCCTCCAGCAGCTTGGGCACCCAGACC | |
| | | TACATCTGCAACGTGAATCACAAGCCCAGCAACACC | |
| | | AAGGTGGACAAGAAAGTTGAGCCCAAATCTTGTGAC | |
| | | AAAACTCACACATGCCCACCGTGCCCAGCACCTGAA | |
| | | CTCCTGGGGGGACCGTCAGTCTTCCTCTTCCCCCCA | |
| | | AAACCCAAGGACACCCTCATGATCTCCCGGACCCCT | |
| | | GAGGTCACATGCGTGGTGGTGGACGTGAGCCACGAA | |
| | | GACCCTGAGGTCAAGTTCAACTGGTACGTGGACGGC | |
| | | GTGGAGGTGCATAATGCCAAGACAAAGCCGCGGGAG | |
| | | GAGCAGTACAACAGCACGTACCGTGTGGTCAGCGTC | |
| | | CTCACCGTCCTGCACCAGGACTGGCTGAATGGCAAG | |
| | | GAGTACAAGTGCAAGGTCTCCAACAAAGCCCTCCCA | |
| | | GCCCCCATCGAGAAAACCATCTCCAAAGCCAAAGGG | |
| | | CAGCCCCGAGAACCACAGGTGTACACCCTGCCCCCA | |
| | | TCCCGGGAGGAGATGACCAAGAACCAGGTCAGCCTG | |
| | | ACCTGCCTGGTCAAAGGCTTCTATCCCAGCGACATC | |
| | | GCCGTGGAGTGGGAGAGCAATGGGCAGCCGGAGAAC | |
| | | AACTACAAGACCACGCCTCCCGTGCTGGACTCCGAC | |
| | | GGCTCCTTCTTCCTCTACAGCAAGCTCACCGTGGAC | |
| | | AAGAGCAGGTGGCAGCAGGGGAACGTCTTCTCATGC | |
| | | TCCGTGATGCATGAGGCTCTGCACAACCACTACACG | |
| | | CAGAAGAGCCTCTCCCTGTCTCCGGGTAAA | |
| 1789 | Heavy | GAGGTTCAGCTGGTGGAGTCTGGCGGTGGCCTGGTG | 33 |
| AM39 | Chain | CAGCCAGGGGGCTCACTCCGTTTGTCCTGTGCAGCT | |
| | | TCTGGCTTCAACGTCTCTTATTCTTCTATACACTGG | |
| | | GTGCGTCAGGCCCCGGGTAAGGGCCTGGAATGGGTT | |
| | | GCAAGTATTAGAGGAGCCGGTAGTGACACTCGCTAT | |
| | | GCCGATAGCGTCAAGGGCCGTTTCACTATAAGCGCA | |
| | | GACACATCCAAAAACACAGCCTACCTACAAATGAAC | |
| | | AGCTTAAGAGCTGAGGACACTGCCGTCTATTATTGT | |
| | | GCTCGCAACTACTGGGGTTGGTACCCGTTCTACTAC | |
| | | TTCGCTTTTGACTACTGGGGTCAAGGAACCCTGGTC | |
| | | ACCGTCTCCTCGGCTAGCACCAAGGGCCCATCGGTC | |
| | | TTCCCCCTGGCACCCTCCTCCAAGAGCACCTCTGGG | |
| | | GGCACAGCGGCCCTGGGCTGCCTGGTCAAGGACTAC | |
| | | TTCCCCGAACCGGTGACGGTGTCGTGGAACTCAGGC | |
| | | GCCCTGACCAGCGGCGTGCACACCTTCCCGGCTGTC | |
| | | CTACAGTCCTCAGGACTCTACTCCCTCAGCAGCGTG | |
| | | GTGACCGTGCCCTCCAGCAGCTTGGGCACCCAGACC | |
| | | TACATCTGCAACGTGAATCACAAGCCCAGCAACACC | |
| | | AAGGTGGACAAGAAAGTTGAGCCCAAATCTTGTGAC | |
| | | AAAACTCACACATGCCCACCGTGCCCAGCACCTGAA | |
| | | CTCCTGGGGGGACCGTCAGTCTTCCTCTTCCCCCCA | |
| | | AAACCCAAGGACACCCTCATGATCTCCCGGACCCCT | |
| | | GAGGTCACATGCGTGGTGGTGGACGTGAGCCACGAA | |
| | | GACCCTGAGGTCAAGTTCAACTGGTACGTGGACGGC | |
| | | GTGGAGGTGCATAATGCCAAGACAAAGCCGCGGGAG | |
| | | GAGCAGTACAACAGCACGTACCGTGTGGTCAGCGTC | |
| | | CTCACCGTCCTGCACCAGGACTGGCTGAATGGCAAG | |
| | | GAGTACAAGTGCAAGGTCTCCAACAAAGCCCTCCCA | |
| | | GCCCCCATCGAGAAAACCATCTCCAAAGCCAAAGGG | |
| | | CAGCCCCGAGAACCACAGGTGTACACCCTGCCCCCA | |
| | | TCCCGGGAGGAGATGACCAAGAACCAGGTCAGCCTG | |
| | | ACCTGCCTGGTCAAAGGCTTCTATCCCAGCGACATC | |
| | | GCCGTGGAGTGGGAGAGCAATGGGCAGCCGGAGAAC | |
| | | AACTACAAGACCACGCCTCCCGTGCTGGACTCCGAC | |
| | | GGCTCCTTCTTCCTCTACAGCAAGCTCACCGTGGAC | |
| | | AAGAGCAGGTGGCAGCAGGGGAACGTCTTCTCATGC | |
| | | TCCGTGATGCATGAGGCTCTGCACAACCACTACACG | |
| | | CAGAAGAGCCTCTCCCTGTCTCCGGGTAAA | |
| 1789 | Heavy | GAGGTTCAGCTGGTGGAGTCTGGCGGTGGCCTGGTG | 34 |
| AM42 | Chain | CAGCCAGGGGGCTCACTCCGTTTGTCCTGTGCAGCT | |
| | | TCTGGCTTCAACGTCTCTTATTCTTCTATACACTGG | |
| | | GTGCGTCAGGCCCCGGGTAAGGGCCTGGAATGGGTT | |
| | | GCAAAAATTCGAGGAGGCCATCAGGACACTCACTAT | |
| | | GCCGATAGCGTCAAGGGCCGTTTCACTATAAGCGCA | |
| | | GACACATCCAAAAACACAGCCTACCTACAAATGAAC | |
| | | AGCTTAAGAGCTGAGGACACTGCCGTCTATTATTGT | |
| | | GCTCGCAACTACTGGGGTTGGTACCCGTTCTACTAC | |
| | | TTCGCTTTTGACTACTGGGGTCAAGGAACCCTGGTC | |
| | | ACCGTCTCCTCGGCTAGCACCAAGGGCCCATCGGTC | |
| | | TTCCCCCTGGCACCCTCCTCCAAGAGCACCTCTGGG | |
| | | GGCACAGCGGCCCTGGGCTGCCTGGTCAAGGACTAC | |
| | | TTCCCCGAACCGGTGACGGTGTCGTGGAACTCAGGC | |
| | | GCCCTGACCAGCGGCGTGCACACCTTCCCGGCTGTC | |
| | | CTACAGTCCTCAGGACTCTACTCCCTCAGCAGCGTG | |
| | | GTGACCGTGCCCTCCAGCAGCTTGGGCACCCAGACC | |
| | | TACATCTGCAACGTGAATCACAAGCCCAGCAACACC | |
| | | AAGGTGGACAAGAAAGTTGAGCCCAAATCTTGTGAC | |
| | | AAAACTCACACATGCCCACCGTGCCCAGCACCTGAA | |
| | | CTCCTGGGGGGACCGTCAGTCTTCCTCTTCCCCCCA | |
| | | AAACCCAAGGACACCCTCATGATCTCCCGGACCCCT | |
| | | GAGGTCACATGCGTGGTGGTGGACGTGAGCCACGAA | |
| | | GACCCTGAGGTCAAGTTCAACTGGTACGTGGACGGC | |
| | | GTGGAGGTGCATAATGCCAAGACAAAGCCGCGGGAG | |
| | | GAGCAGTACAACAGCACGTACCGTGTGGTCAGCGTC | |
| | | CTCACCGTCCTGCACCAGGACTGGCTGAATGGCAAG | |
| | | GAGTACAAGTGCAAGGTCTCCAACAAAGCCCTCCCA | |
| | | GCCCCCATCGAGAAAACCATCTCCAAAGCCAAAGGG | |
| | | CAGCCCCGAGAACCACAGGTGTACACCCTGCCCCCA | |
| | | TCCCGGGAGGAGATGACCAAGAACCAGGTCAGCCTG | |
| | | ACCTGCCTGGTCAAAGGCTTCTATCCCAGCGACATC | |
| | | GCCGTGGAGTGGGAGAGCAATGGGCAGCCGGAGAAC | |
| | | AACTACAAGACCACGCCTCCCGTGCTGGACTCCGAC | |
| | | GGCTCCTTCTTCCTCTACAGCAAGCTCACCGTGGAC | |
| | | AAGAGCAGGTGGCAGCAGGGGAACGTCTTCTCATGC | |
| | | TCCGTGATGCATGAGGCTCTGCACAACCACTACACG | |
| | | CAGAAGAGCCTCTCCCTGTCTCCGGGTAAA | |
| 1798 | Heavy | GAGGTTCAGCTGGTGGAGTCTGGCGGTGGCCTGGTG | 35 |
| | Chain | CAGCCAGGGGGCTCACTCCGTTTGTCCTGTGCAGCT | |
| | | TCTGGCTTCAACTTCTCTTATTCTTCTATACACTGG | |
| | | GTGCGTCAGGCCCCGGGTAAGGGCCTGGAATGGGTT | |
| | | GCATCTATTTCTTCTTCTTATGGCTCTACTTATTAT | |
| | | GCCGATAGCGTCAAGGGCCGTTTCACTATAAGCGCA | |
| | | GACACATCCAAAAACACAGCCTACCTACAAATGAAC | |
| | | AGCTTAAGAGCTGAGGACACTGCCGTCTATTATTGT | |
| | | GCTCGCGCTTTCTACTCTCATGACTACCGTTACATC | |
| | | TACTGGGGTTCTGGTATGGACTACTGGGGTCAAGGA | |
| | | ACCCTGGTCACCGTCTCCTCGGCTAGCACCAAGGGC | |
| | | CCATCGGTCTTCCCCCTGGCACCCTCCTCCAAGAGC | |
| | | ACCTCTGGGGGCACAGCGGCCCTGGGCTGCCTGGTC | |
| | | AAGGACTACTTCCCCGAACCGGTGACGGTGTCGTGG | |
| | | AACTCAGGCGCCCTGACCAGCGGCGTGCACACCTTC | |
| | | CCGGCTGTCCTACAGTCCTCAGGACTCTACTCCCTC | |
| | | AGCAGCGTGGTGACCGTGCCCTCCAGCAGCTTGGGC | |
| | | ACCCAGACCTACATCTGCAACGTGAATCACAAGCCC | |
| | | AGCAACACCAAGGTGGACAAGAAAGTTGAGCCCAAA | |
| | | TCTTGTGACAAAACTCACACATGCCCACCGTGCCCA | |
| | | GCACCTGAACTCCTGGGGGGACCGTCAGTCTTCCTC | |
| | | TTCCCCCCAAAACCCAAGGACACCCTCATGATCTCC | |
| | | CGGACCCCTGAGGTCACATGCGTGGTGGTGGACGTG | |
| | | AGCCACGAAGACCCTGAGGTCAAGTTCAACTGGTAC | |
| | | GTGGACGGCGTGGAGGTGCATAATGCCAAGACAAAG | |
| | | CCGCGGGAGGAGCAGTACAACAGCACGTACCGTGTG | |
| | | GTCAGCGTCCTCACCGTCCTGCACCAGGACTGGCTG | |
| | | AATGGCAAGGAGTACAAGTGCAAGGTCTCCAACAAA | |
| | | GCCCTCCCAGCCCCCATCGAGAAAACCATCTCCAAA | |
| | | GCCAAAGGGCAGCCCCGAGAACCACAGGTGTACACC | |
| | | CTGCCCCCATCCCGGGAGGAGATGACCAAGAACCAG | |
| | | GTCAGCCTGACCTGCCTGGTCAAAGGCTTCTATCCC | |
| | | AGCGACATCGCCGTGGAGTGGGAGAGCAATGGGCAG | |
| | | CCGGAGAACAACTACAAGACCACGCCTCCCGTGCTG | |
| | | GACTCCGACGGCTCCTTCTTCCTCTACAGCAAGCTC | |
| | | ACCGTGGACAAGAGCAGGTGGCAGCAGGGGAACGTC | |
| | | TTCTCATGCTCCGTGATGCATGAGGCTCTGCACAAC | |
| | | CACTACACGCAGAAGAGCCTCTCCCTGTCTCCGGGT | |
| | | AAA | |
| 1557 | Heavy | GAGGTTCAGCTGGTGGAGTCTGGCGGTGGCCTGGTG | 36 |
| | Chain | CAGCCAGGGGGCTCACTCCGTTTGTCCTGTGCAGCT | |
| | | TCTGGCTTCAACGTCTATTATTCTTCTATACACTGG | |
| | | GTGCGTCAGGCCCCGGGTAAGGGCCTGGAATGGGTT | |
| | | GCATCTATTTCTTCTTCTTCTGGCTCTACTTCTTAT | |
| | | GCCGATAGCGTCAAGGGCCGTTTCACTATAAGCGCA | |
| | | GACACATCCAAAAACACAGCCTACCTACAAATGAAC | |
| | | AGCTTAAGAGCTGAGGACACTGCCGTCTATTATTGT | |
| | | GCTCGCTACGGTTGGTGGGAATACGGTTACGGTGGT | |
| | | TGGTGGCATCCGGCTTTGGACTACTGGGGTCAAGGA | |
| | | ACCCTGGTCACCGTCTCCTCGGCTAGCACCAAGGGC | |
| | | CCATCGGTCTTCCCCCTGGCACCCTCCTCCAAGAGC | |
| | | ACCTCTGGGGGCACAGCGGCCCTGGGCTGCCTGGTC | |
| | | AAGGACTACTTCCCCGAACCGGTGACGGTGTCGTGG | |
| | | AACTCAGGCGCCCTGACCAGCGGCGTGCACACCTTC | |
| | | CCGGCTGTCCTACAGTCCTCAGGACTCTACTCCCTC | |
| | | AGCAGCGTGGTGACCGTGCCCTCCAGCAGCTTGGGC | |
| | | ACCCAGACCTACATCTGCAACGTGAATCACAAGCCC | |
| | | AGCAACACCAAGGTGGACAAGAAAGTTGAGCCCAAA | |
| | | TCTTGTGACAAAACTCACACATGCCCACCGTGCCCA | |
| | | GCACCTGAACTCCTGGGGGGACCGTCAGTCTTCCTC | |
| | | TTCCCCCCAAAACCCAAGGACACCCTCATGATCTCC | |
| | | CGGACCCCTGAGGTCACATGCGTGGTGGTGGACGTG | |
| | | AGCCACGAAGACCCTGAGGTCAAGTTCAACTGGTAC | |
| | | GTGGACGGCGTGGAGGTGCATAATGCCAAGACAAAG | |
| | | CCGCGGGAGGAGCAGTACAACAGCACGTACCGTGTG | |
| | | GTCAGCGTCCTCACCGTCCTGCACCAGGACTGGCTG | |
| | | AATGGCAAGGAGTACAAGTGCAAGGTCTCCAACAAA | |
| | | GCCCTCCCAGCCCCCATCGAGAAAACCATCTCCAAA | |
| | | GCCAAAGGGCAGCCCCGAGAACCACAGGTGTACACC | |
| | | CTGCCCCCATCCCGGGAGGAGATGACCAAGAACCAG | |
| | | GTCAGCCTGACCTGCCTGGTCAAAGGCTTCTATCCC | |
| | | AGCGACATCGCCGTGGAGTGGGAGAGCAATGGGCAG | |
| | | CCGGAGAACAACTACAAGACCACGCCTCCCGTGCTG | |
| | | GACTCCGACGGCTCCTTCTTCCTCTACAGCAAGCTC | |
| | | ACCGTGGACAAGAGCAGGTGGCAGCAGGGGAACGTC | |
| | | TTCTCATGCTCCGTGATGCATGAGGCTCTGCACAAC | |
| | | CACTACACGCAGAAGAGCCTCTCCCTGTCTCCGGGT | |
| | | AAA | |
| 1780 | Light | TCCGATATCCAGATGACCCAGTCCCCGAGCTCCCTG | 38 |
| | Chain | TCCGCCTCTGTGGGCGATAGGGTCACCATCACCTGC | |
| | | CGTGCCAGTCAGTCCGTGTCCAGCGCTGTAGCCTGG | |
| | | TATCAACAGAAACCAGGAAAAGCTCCGAAGCTTCTG | |
| | | ATTTACTCGGCATCCAGCCTCTACTCTGGAGTCCCT | |
| | | TCTCGCTTCTCTGGTAGCCGTTCCGGGACGGATTTC | |
| | | ACTCTGACCATCAGCAGTCTGCAGCCGGAAGACTTC | |
| | | GCAACTTATTACTGTCAGCAAGCTCATCCGAAACCG | |
| | | ATCACGTTCGGACAGGGTACCAAGGTGGAGATCAAA | |
| | | CGAACTGTGGCTGCACCATCTGTCTTCATCTTCCCG | |
| | | CCATCTGATTCACAGTTGAAATCTGGAACTGCCTCT | |
| | | GTTGTGTGCCTGCTGAATAACTTCTATCCCAGAGAG | |
| | | GCCAAAGTACAGTGGAAGGTGGATAACGCCCTCCAA | |
| | | TCGGGTAACTCCCAGGAGAGTGTCACAGAGCAGGAC | |
| | | AGCAAGGACAGCACCTACAGCCTCAGCAGCACCCTG | |
| | | ACGCTGAGCAAAGCAGACTACGAAAAACATAAAGTC | |
| | | TACGCCTGCGAAGTCACCCATCAGGGCCTGAGCTCG | |
| | | CCCGTCACAAAGAGCTTCAACAGGGGAGAGTGT | |
| 1789 | Light | TCCGATATCCAGATGACCCAGTCCCCGAGCTCCCTG | 40 |
| | Chain | TCCGCCTCTGTGGGCGATAGGGTCACCATCACCTGC | |
| | | CGTGCCAGTCAGTCCGTGTCCAGCGCTGTAGCCTGG | |
| | | TATCAACAGAAACCAGGAAAAGCTCCGAAGCTTCTG | |
| | | ATTTACTCGGCATCCAGCCTCTACTCTGGAGTCCCT | |
| | | TCTCGCTTCTCTGGTAGCCGTTCCGGGACGGATTTC | |
| | | ACTCTGACCATCAGCAGTCTGCAGCCGGAAGACTTC | |
| | | GCAACTTATTACTGTCAGCAAGGTTGGGACTACCCG | |
| | | ATCACGTTCGGACAGGGTACCAAGGTGGAGATCAAA | |
| | | CGAACTGTGGCTGCACCATCTGTCTTCATCTTCCCG | |
| | | CCATCTGATTCACAGTTGAAATCTGGAACTGCCTCT | |
| | | GTTGTGTGCCTGCTGAATAACTTCTATCCCAGAGAG | |
| | | GCCAAAGTACAGTGGAAGGTGGATAACGCCCTCCAA | |
| | | TCGGGTAACTCCCAGGAGAGTGTCACAGAGCAGGAC | |
| | | AGCAAGGACAGCACCTACAGCCTCAGCAGCACCCTG | |
| | | ACGCTGAGCAAAGCAGACTACGAAAAACATAAAGTC | |
| | | TACGCCTGCGAAGTCACCCATCAGGGCCTGAGCTCG | |
| | | CCCGTCACAAAGAGCTTCAACAGGGGAGAGTGT | |
| 1789 | Light | TCCGATATCCAGATGACCCAGTCCCCGAGCTCCCTG | 41 |
| AM32 | Chain | TCCGCCTCTGTGGGCGATAGGGTCACCATCACCTGC | |
| | | CGTGCCAGTCAGTCCGTGTCCAGCGCTGTAGCCTGG | |
| | | TATCAACAGAAACCAGGAAAAGCTCCGAAGCTTCTG | |
| | | ATTTACTCGGCATCCAGCCTCTACTCTGGAGTCCCT | |
| | | TCTCGCTTCTCTGGTAGCCGTTCCGGGACGGATTTC | |
| | | ACTCTGACCATCAGCAGTCTGCAGCCGGAAGACTTC | |
| | | GCAACTTATTACTGTCAGCAAGGTTGGGACTACCCG | |
| | | ATCACGTTCGGACAGGGTACCAAGGTGGAGATCAAA | |
| | | CGAACTGTGGCTGCACCATCTGTCTTCATCTTCCCG | |
| | | CCATCTGATTCACAGTTGAAATCTGGAACTGCCTCT | |
| | | GTTGTGTGCCTGCTGAATAACTTCTATCCCAGAGAG | |
| | | GCCAAAGTACAGTGGAAGGTGGATAACGCCCTCCAA | |
| | | TCGGGTAACTCCCAGGAGAGTGTCACAGAGCAGGAC | |
| | | AGCAAGGACAGCACCTACAGCCTCAGCAGCACCCTG | |
| | | ACGCTGAGCAAAGCAGACTACGAAAAACATAAAGTC | |
| | | TACGCCTGCGAAGTCACCCATCAGGGCCTGAGCTCG | |
| | | CCCGTCACAAAGAGCTTCAACAGGGGAGAGTGT | |
| 1789 | Light | TCCGATATCCAGATGACCCAGTCCCCGAGCTCCCTG | 43 |
| AM39 | Chain | TCCGCCTCTGTGGGCGATAGGGTCACCATCACCTGC | |
| | | CGTGCCAGTCAGTCCGTGTCCAGCGCTGTAGCCTGG | |
| | | TATCAACAGAAACCAGGAAAAGCTCCGAAGCTTCTG | |
| | | ATTTACTCGGCATCCAGCCTCTACTCTGGAGTCCCT | |
| | | TCTCGCTTCTCTGGTAGCCGTTCCGGGACGGATTTC | |
| | | ACTCTGACCATCAGCAGTCTGCAGCCGGAAGACTTC | |
| | | GCAACTTATTACTGTCAGCAAGGTTGGGACTACCCG | |
| | | ATCACGTTCGGACAGGGTACCAAGGTGGAGATCAAA | |
| | | CGAACTGTGGCTGCACCATCTGTCTTCATCTTCCCG | |
| | | CCATCTGATTCACAGTTGAAATCTGGAACTGCCTCT | |
| | | GTTGTGTGCCTGCTGAATAACTTCTATCCCAGAGAG | |
| | | GCCAAAGTACAGTGGAAGGTGGATAACGCCCTCCAA | |
| | | TCGGGTAACTCCCAGGAGAGTGTCACAGAGCAGGAC | |
| | | AGCAAGGACAGCACCTACAGCCTCAGCAGCACCCTG | |
| | | ACGCTGAGCAAAGCAGACTACGAAAAACATAAAGTC | |
| | | TACGCCTGCGAAGTCACCCATCAGGGCCTGAGCTCG | |
| | | CCCGTCACAAAGAGCTTCAACAGGGGAGAGTGT | |
| 1789 | Light | TCCGATATCCAGATGACCCAGTCCCCGAGCTCCCTG | 44 |
| AM42 | Chain | TCCGCCTCTGTGGGCGATAGGGTCACCATCACCTGC | |
| | | CGTGCCAGTCAGTCCGTGTCCAGCGCTGTAGCCTGG | |
| | | TATCAACAGAAACCAGGAAAAGCTCCGAAGCTTCTG | |
| | | ATTTACTCGGCATCCAGCCTCTACTCTGGAGTCCCT | |
| | | TCTCGCTTCTCTGGTAGCCGTTCCGGGACGGATTTC | |
| | | ACTCTGACCATCAGCAGTCTGCAGCCGGAAGACTTC | |
| | | GCAACTTATTACTGTCAGCAAGGTTGGGACTACCCG | |
| | | ATCACGTTCGGACAGGGTACCAAGGTGGAGATCAAA | |
| | | CGAACTGTGGCTGCACCATCTGTCTTCATCTTCCCG | |
| | | CCATCTGATTCACAGTTGAAATCTGGAACTGCCTCT | |
| | | GTTGTGTGCCTGCTGAATAACTTCTATCCCAGAGAG | |
| | | GCCAAAGTACAGTGGAAGGTGGATAACGCCCTCCAA | |
| | | TCGGGTAACTCCCAGGAGAGTGTCACAGAGCAGGAC | |
| | | AGCAAGGACAGCACCTACAGCCTCAGCAGCACCCTG | |
| | | ACGCTGAGCAAAGCAGACTACGAAAAACATAAAGTC | |
| | | TACGCCTGCGAAGTCACCCATCAGGGCCTGAGCTCG | |
| | | CCCGTCACAAAGAGCTTCAACAGGGGAGAGTGT | |
| 1798 | Light | TCCGATATCCAGATGACCCAGTCCCCGAGCTCCCTG | 45 |
| | Chain | TCCGCCTCTGTGGGCGATAGGGTCACCATCACCTGC | |
| | | CGTGCCAGTCAGTCCGTGTCCAGCGCTGTAGCCTGG | |
| | | TATCAACAGAAACCAGGAAAAGCTCCGAAGCTTCTG | |
| | | ATTTACTCGGCATCCAGCCTCTACTCTGGAGTCCCT | |
| | | TCTCGCTTCTCTGGTAGCCGTTCCGGGACGGATTTC | |
| | | ACTCTGACCATCAGCAGTCTGCAGCCGGAAGACTTC | |
| | | GCAACTTATTACTGTCAGCAATCTTGGTACTACCCG | |
| | | TTCACGTTCGGACAGGGTACCAAGGTGGAGATCAAA | |
| | | CGAACTGTGGCTGCACCATCTGTCTTCATCTTCCCG | |
| | | CCATCTGATTCACAGTTGAAATCTGGAACTGCCTCT | |
| | | GTTGTGTGCCTGCTGAATAACTTCTATCCCAGAGAG | |
| | | GCCAAAGTACAGTGGAAGGTGGATAACGCCCTCCAA | |
| | | TCGGGTAACTCCCAGGAGAGTGTCACAGAGCAGGAC | |
| | | AGCAAGGACAGCACCTACAGCCTCAGCAGCACCCTG | |
| | | ACGCTGAGCAAAGCAGACTACGAAAAACATAAAGTC | |
| | | TACGCCTGCGAAGTCACCCATCAGGGCCTGAGCTCG | |
| | | CCCGTCACAAAGAGCTTCAACAGGGGAGAGTGT | |
| 1557 | Light | TCCGATATCCAGATGACCCAGTCCCCGAGCTCCCTG | 48 |
| | Chain | TCCGCCTCTGTGGGCGATAGGGTCACCATCACCTGC | |
| | | CGTGCCAGTCAGTCCGTGTCCAGCGCTGTAGCCTGG | |
| | | TATCAACAGAAACCAGGAAAAGCTCCGAAGCTTCTG | |
| | | ATTTACTCGGCATCCAGCCTCTACTCTGGAGTCCCT | |
| | | TCTCGCTTCTCTGGTAGCCGTTCCGGGACGGATTTC | |
| | | ACTCTGACCATCAGCAGTCTGCAGCCGGAAGACTTC | |
| | | GCAACTTATTACTGTCAGCAAGTTTGGTGGGGTCCG | |
| | | ATCACGTTCGGACAGGGTACCAAGGTGGAGATCAAA | |
| | | CGAACTGTGGCTGCACCATCTGTCTTCATCTTCCCG | |
| | | CCATCTGATTCACAGTTGAAATCTGGAACTGCCTCT | |
| | | GTTGTGTGCCTGCTGAATAACTTCTATCCCAGAGAG | |
| | | GCCAAAGTACAGTGGAAGGTGGATAACGCCCTCCAA | |
| | | TCGGGTAACTCCCAGGAGAGTGTCACAGAGCAGGAC | |
| | | AGCAAGGACAGCACCTACAGCCTCAGCAGCACCCTG | |
| | | ACGCTGAGCAAAGCAGACTACGAAAAACATAAAGTC | |
| | | TACGCCTGCGAAGTCACCCATCAGGGCCTGAGCTCG | |
| | | CCCGTCACAAAGAGCTTCAACAGGGGAGAGTGT | |

TABLE 3
Summary of SEQ ID NOs.

| | HCDR | HCDR | HCDR | LCDR | LCDR | LCDR | | | Heavy | Light |
|---|---|---|---|---|---|---|---|---|---|---|
| Clone | 1 | 2 | 3 | 1 | 2 | 3 | VH | VL | Chain | Chain |
| 1780 | 1 | 2 | 3 | 4 | 5 | 6 | 7 | 8 | 9 | 10 |
| 1789 | 11 | 2 | 13 | 4 | 5 | 16 | 17 | 18 | 19 | 20 |
| 1789 | 21 | 22 | 13 | 4 | 5 | 16 | 27 | 18 | 29 | 30 |
| AM32 | | | | | | | | | | |
| 1789 | 21 | 32 | 13 | 4 | 5 | 16 | 37 | 18 | 39 | 30 |
| AM39 | | | | | | | | | | |
| 1789 | 21 | 42 | 13 | 4 | 5 | 46 | 47 | 18 | 49 | 30 |
| AM42 | | | | | | | | | | |
| 1798 | 51 | 52 | 53 | 4 | 5 | 56 | 57 | 58 | 59 | 60 |
| 1557 | 61 | 62 | 12 | 4 | 5 | 14 | 15 | 23 | 24 | 25 |

### 1. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

### 2. Considerations for Substitutions

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within ±2 are included, in other embodiments, those which are within ±1 are included, and in still other embodiments, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some embodiments of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

### VI. NUCLEIC ACIDS

In certain embodiments, nucleic acid sequences can exist in a variety of instances such as: isolated segments and recombinant vectors of incorporated sequences or recombinant polynucleotides encoding peptides and polypeptides of the disclosure, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### A. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### B. Mutation

Changes can be introduced by mutation into a nucleic acid, thereby leading to changes in the amino acid sequence of a polypeptide (e.g., an antigenic peptide or polypeptide) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### C. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another embodiment, the nucleic acid molecules may be used as probes or PCR primers for specific nucleic acid sequences. For instance, a nucleic acid molecule probe may be used in diagnostic methods or a nucleic acid molecule PCR primer may be used to amplify regions of DNA that could be used, inter alia, to isolate nucleic acid sequences for use in producing the engineered cells of the disclosure. In a preferred embodiment, the nucleic acid molecules are oligonucleotides.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### VII. POLYPEPTIDE EXPRESSION

In some aspects, there are nucleic acid molecule encoding polypeptides, antibodies, or antigen binding fragments of the disclosure. The nucleic acid molecules may be used to express large quantities of polypeptides. If the nucleic acid molecules are derived from a non-human, non-transgenic animal, the nucleic acid molecules may be used for humanization of the antibody or TCR genes.

### A. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody heavy and/or light chain, antibody fragments, and probes thereof. In addition to control sequences that govern transcription and translation, vectors and expression vectors may contain nucleic acid sequences that serve other functions as well.

To express the polypeptides or peptides of the disclosure, DNAs encoding the polypeptides or peptides are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. In some aspects, a vector that encodes a functionally complete human TCR alpha or TCR beta sequence with appropriate restriction sites engineered so that any variable sequence or CDR1, CDR2, and/or CDR3 can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### B. Expression Systems

Numerous expression systems exist that comprise at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### C. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### VIII. CANCER THERAPY

In some aspects, the disclosed methods comprise administering a cancer therapy to a subject or patient. The cancer therapy may be chosen based on an expression level measurement, alone or in combination with the clinical risk score calculated for the subject. The cancer therapy may be chosen based on a genotype of a subject. The cancer therapy may be chosen based on the presence or absence of one or more polymorphisms in a subject. In some aspects, the cancer therapy comprises a local cancer therapy. In some aspects, the cancer therapy excludes a systemic cancer therapy. In some aspects, the cancer therapy excludes a local therapy. In some aspects, the cancer therapy comprises a local cancer therapy without the administration of a system cancer therapy. In some aspects, the cancer therapy comprises an immunotherapy, which may be a checkpoint inhibitor therapy. Any of these cancer therapies may also be excluded. Combinations of these therapies may also be administered.

The term “cancer,” as used herein, may be used to describe a solid tumor, metastatic cancer, or non-metastatic cancer. In certain aspects, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some aspects, the cancer is a Stage I cancer. In some aspects, the cancer is a Stage II cancer. In some aspects, the cancer is a Stage III cancer. In some aspects, the cancer is a Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some aspects, the cancer is aggressive cancer. In some aspects, the cancer is Stage I cancer. In some aspects, the cancer is Stage II cancer (e.g., IIA, IIB, IIC). In some aspects, the cancer is Stage III cancer (e.g., IIIA, IIIB, IIIC). In some aspects, the cancer is Stage IV cancer (e.g., IVA, IVB).

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

### A. NT5E-Targeting Polypeptides

Aspects of the disclosure comprise administering one or more NT5E-targeting polypeptides of the present disclosure to a subject. A subject may be administered 1, 2, 3, 4, 5, 6, 7, 8, 9, 10, or more NT5E-targeting polypeptides of the disclosure. A NT5E-targeting polypeptide of the disclosure may be administered alone or in combination with any other cancer therapy known in the art and/or described herein. A NT5E-targeting polypeptide of the disclosure may be administered to a subject in various forms, including as a recombinant polypeptide, as a nucleic acid encoding the polypeptide, and as a cell (e.g., immune cell such as T cell or NK cell) expressing or capable of expressing the polypeptide. As used herein, “NT5E-targeting polypeptide” and “CD73-targeting polypeptide” are used interchangeably.

### B. Radiotherapy

In some aspects, a radiotherapy, such as ionizing radiation, is administered to a subject. As used herein, “ionizing radiation” means radiation comprising particles or photons that have sufficient energy or can produce sufficient energy via nuclear interactions to produce ionization (gain or loss of electrons). A non-limiting example of ionizing radiation is x-radiation. Means for delivering x-radiation to a target tissue or cell are well known in the art.

In some aspects, the radiotherapy can comprise external radiotherapy, internal radiotherapy, radioimmunotherapy, or intraoperative radiation therapy (IORT). In some aspects, the external radiotherapy comprises three-dimensional conformal radiation therapy (3D-CRT), intensity modulated radiation therapy (IMRT), proton beam therapy, image-guided radiation therapy (IGRT), or stereotactic radiation therapy. In some aspects, the internal radiotherapy comprises interstitial brachytherapy, intracavitary brachytherapy, or intraluminal radiation therapy. In some aspects, the radiotherapy is administered to a primary tumor.

In some aspects, the amount of ionizing radiation is greater than 20 Gy and is administered in one dose. In some aspects, the amount of ionizing radiation is 18 Gy and is administered in three doses. In some aspects, the amount of ionizing radiation is at least, at most, or exactly 0.5, 1, 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 Gy (or any derivable range therein). In some aspects, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some aspects, the amount of radiotherapy administered to a subject may be presented as a total dose of radiotherapy, which is then administered in fractionated doses. For example, in some aspects, the total dose is 50 Gy administered in 10 fractionated doses of 5 Gy each. In some aspects, the total dose is 50-90 Gy, administered in 20-60 fractionated doses of 2-3 Gy each. In some aspects, the total dose of radiation is at least, at most, or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 Gy (or any derivable range therein). In some aspects, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein). In some aspects, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some aspects, at least, at most, or exactly 1, 2, 3, 4, 5, 6, 7, 8, 9, 10, 11, or 12 (or any derivable range therein) fractionated doses are administered per day. In some aspects, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### C. Cancer Immunotherapy

In some aspects, the methods comprise administration of a cancer immunotherapy. Cancer immunotherapy (sometimes called immuno-oncology, abbreviated IO) is the use of the immune system to treat cancer. Immunotherapies can, in some cases, be categorized as active, passive, or hybrid (active and passive). These approaches may exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and certain examples are described below.

### 1. Checkpoint Inhibitors and Combination Treatment

Aspects of the disclosure may include administration of immune checkpoint inhibitors, examples of which are further described below. As disclosed herein, “checkpoint inhibitor therapy” (also “immune checkpoint blockade therapy”, “immune checkpoint therapy”, “ICT,” “checkpoint blockade immunotherapy,” or “CBI”), refers to cancer therapy comprising providing one or more immune checkpoint inhibitors to a subject suffering from or suspected of having cancer.

a. PD-1, PDL1, and PDL2 inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some aspects, PD-1, PDL1, and PDL2 are human PD-1, PDL1 and PDL2.

In some aspects, the PD-1 inhibitor is a molecule that inhibits the binding of PD-1 to its ligand binding partners. In a specific aspect, the PD-1 ligand binding partners are PDL1 and/or PDL2. In another aspect, a PDL1 inhibitor is a molecule that inhibits the binding of PDL1 to its binding partners. In a specific aspect, PDL1 binding partners are PD-1 and/or B7-1. In another aspect, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some aspects, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDIO680, also known as AMP-514, and REGN2810.

In some aspects, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the VH region of nivolumab, pembrolizumab, or pidilizumab, and the CDR1, CDR2 and CDR3 domains of the V_{L} region of nivolumab, pembrolizumab, or pidilizumab. In another aspect, the antibody competes for binding with and/or binds to the same epitope on PD-1, PDL1, or PDL2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or V_{H} and/or V_{L} domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 1ODI, MDX-010, MDX-101, and Yervoy®) or antigen binding fragments and variants thereof (see, e.g., WO 01/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the V_{H} region of tremelimumab or ipilimumab, and the CDR1, CDR2 and CDR3 domains of the V_{L} region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

c. LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8+ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some aspects, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or V_{H} and/or V_{L} domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4(Supp. 1):P195 (REGN3767); Yu, X., et al., mAbs, 2019; 11:6 (LBL-007) can be used in the methods disclosed herein. These and other anti-LAG-3 antibodies useful in the claimed invention can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some aspects, the inhibitor comprises the heavy and light chain CDRs or VRs of an anti-LAG3 antibody. Accordingly, in one aspect, the inhibitor comprises the CDR1, CDR2, and CDR3 domains of the V_{H} region of an anti-LAG3 antibody, and the CDR1, CDR2 and CDR3 domains of the V_{L} region of an anti-LAG3 antibody. In another aspect, the antibody has at least about 70, 75, 80, 85, 90, 95, 97, or 99% (or any derivable range therein) variable region amino acid sequence identity with the above-mentioned antibodies.

d. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4+Th1 and CD8+Tc1 cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+Th1-specific cell surface protein that regulates macrophage activation. Inhibitors of the disclosure may block one or more functions of TIM-3 activity.

In some aspects, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or V_{H} and/or V_{L} domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed invention can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to TIM-3 also can be used.

In some aspects, the inhibitor comprises the heavy and light chain CDRs or VRs of an anti-TIM-3 antibody. Accordingly, in one aspect, the inhibitor comprises the CDR1, CDR2, and CDR3 domains of the V_{H} region of an anti-TIM-3 antibody, and the CDR1, CDR2 and CDR3 domains of the V_{L} region of an anti-TIM-3 antibody. In another aspect, the antibody has at least about 70, 75, 80, 85, 90, 95, 97, or 99% (or any derivable range or value therein) variable region amino acid sequence identity with the above-mentioned antibodies.

### 2. Activator of Co-Stimulatory Molecules

In some aspects, the immunotherapy comprises an activator (also “agonist”) of a co-stimulatory molecule. In some aspects, the agonist comprises an agonist of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Agonists include activating antibodies, polypeptides, compounds, and nucleic acids.

### 3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell, natural killer (NK) cell, or other immune cell. The receptors are called chimeric because they are fused of parts from different sources. CAR-T cell therapy refers to a treatment that uses such transformed cells for cancer therapy, where the transformed cells are T cells. Similar therapies include, for example, CAR-NK cell therapy, which uses transformed NK cells.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Example CAR-T therapies include Tisagenlecleucel (Kymriah) and Axicabtagene ciloleucel (Yescarta).

### 5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an example interleukin cytokine therapy.

### 6. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

### D. Oncolytic Virus

In some aspects, the cancer therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy

### E. Chemotherapies

In some aspects, a therapy of the present disclosure comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some aspects, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection.

Other suitable chemotherapeutic agents include antimicrotubule agents, e.g., Paclitaxel (“Taxol”) and doxorubicin hydrochloride (“doxorubicin”). Doxorubicin is absorbed poorly and is preferably administered intravenously. In certain aspects, appropriate intravenous doses for an adult include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred in certain cases. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

The amount of the chemotherapeutic agent delivered to a patient may be variable. In one suitable aspect, the chemotherapeutic agent may be administered in an amount effective to cause arrest or regression of the cancer in a host, when the chemotherapy is administered with the construct. In other aspects, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### F. Hormone Therapy

In some aspects, a cancer therapy of the present disclosure is a hormone therapy. In particular aspects, a prostate cancer therapy comprises hormone therapy. Various hormone therapies are known in the art and contemplated herein. Examples of hormone therapies include, but are not limited to, luteinizing hormone-releasing hormone (LHRH) analogs, LHRH antagonists, androgen receptor antagonists, and androgen synthesis inhibitors.

### G. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### H. Additional Cancer Therapies

Therapeutic methods disclosed herein may comprise one or more additional cancer therapies. A cancer therapy of the disclosure may comprise, for example, cryoablative therapy, high-intensity ultrasound (also “high-intensity focused ultrasound”), photodynamic therapy, laser ablation, and/or irreversible electroporation. A cancer therapy of the disclosure may comprise 1, 2, 3, 4, 5, or more distinct therapeutic methods.

It is contemplated that a cancer treatment may exclude any of the cancer treatments described herein. Furthermore, aspects of the disclosure include patients that have been previously treated for a therapy described herein, are currently being treated for a therapy described herein, or have not been treated for a therapy described herein. In some aspects, the patient is one that has been determined to be resistant to a therapy described herein. In some aspects, the patient is one that has been determined to be sensitive to a therapy described herein.

### IX. PHARMACEUTICAL COMPOSITIONS

The present disclosure includes methods for treating disease and modulating immune responses in a subject in need thereof. The disclosure includes cells that may be in the form of a pharmaceutical composition that can be used to induce or modify an immune response.

Administration of the compositions according to the current disclosure will typically be via any common route. This includes, but is not limited to parenteral, orthotopic, intradermal, subcutaneous, orally, transdermally, intramuscular, intraperitoneal, intraperitoneally, intraorbitally, by implantation, by inhalation, intraventricularly, intranasally or intravenous injection. In some embodiments, compositions of the present disclosure (e.g., compositions comprising NT5E-binding polypeptides) are administered to a subject intravenously.

Typically, compositions and therapies of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most or at least 3, 4, 5, 6, 7, 8, 9, 10 or more. The administrations may range from 2-day to 12-week intervals, more usually from one to two week intervals.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions and the preparations can also be emulsified.

Pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (e.g., polypeptides of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

The compositions and related methods of the present disclosure, particularly administration of a composition of the disclosure may also be used in combination with the administration of additional therapies such as the additional therapeutics described herein or in combination with other traditional therapeutics known in the art.

The therapeutic compositions and treatments disclosed herein may precede, be concurrent with and/or follow another treatment or agent by intervals ranging from minutes to weeks. In embodiments where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some embodiments, the therapeutically effective or sufficient amount of the immune checkpoint inhibitor, such as an antibody and/or microbial modulator, that is administered to a human will be in the range of about 0.01 to about 50 mg/kg of patient body weight whether by one or more administrations. In some embodiments, the therapy used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In one embodiment, a therapy described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### X. DETECTABLE LABELS

In some aspects of this disclosure, it will be useful to detectably or therapeutically label a Fab polypeptide. Methods for conjugating polypeptides to these agents are known in the art. For the purpose of illustration only, polypeptides can be labeled with a detectable moiety such as a radioactive atom, a chromophore, a fluorophore, or the like. Such labeled polypeptides can be used for diagnostic techniques, either in vivo, or in an isolated test sample or in methods described herein.

As used herein, the term “label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition that is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6.sup.th ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6.sup.th ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/polypeptides, e.g., rhodamine/anti-rhodamine, biotin/avidin and biotin/strepavidin.

The coupling of polypeptides to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten polypeptides. See, Harlow and Lane (1988) supra.

### XI. SAMPLE PREPARATION

In certain aspects, methods involve obtaining or evaluating a sample from a subject. The sample may include a sample obtained from any source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples may be obtained for diagnosis by the methods described herein. In other cases, multiple samples, such as one or more samples from one tissue type (for example esophagus, lung, breast, etc.) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### XII. HOST CELLS

As used herein, the terms “cell,” “cell line,” and “cell culture” may be used interchangeably. All of these terms also include both freshly isolated cells and ex vivo cultured, activated or expanded cells. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

In certain embodiments transfection can be carried out on any prokaryotic or eukaryotic cell. In some aspects electroporation involves transfection of a human cell. In other aspects electroporation involves transfection of an animal cell. In certain aspects transfection involves transfection of a cell line or a hybrid cell type. In some aspects the cell or cells being transfected are cancer cells, tumor cells or immortalized cells. In some instances tumor, cancer, immortalized cells or cell lines are induced and in other instances tumor, cancer, immortalized cells or cell lines enter their respective state or condition naturally. In certain aspects the cells or cell lines can be A549, B-cells, B16, BHK-21, C2C12, C6, CaCo-2, CAP/, CAP-T, CHO, CHO2, CHO-DG44, CHO-K1, COS-1, Cos-7, CV-1, Dendritic cells, DLD-1, Embryonic Stem (ES) Cell or derivative, H1299, HEK, 293, 293T, 293FT, Hep G2, Hematopoietic Stem Cells, HOS, Huh-7, Induced Pluripotent Stem (iPS) Cell or derivative, Jurkat, K562, L5278Y, LNCaP, MCF7, MDA-MB-231, MDCK, Mesenchymal Cells, Min-6, Monocytic cell, Neuro2a, NIH 3T3, NIH3T3L1, K562, NK-cells, NSO, Panc-1, PC12, PC-3, Peripheral blood cells, Plasma cells, Primary Fibroblasts, RBL, Renca, RLE, SF21, SF9, SH-SY5Y, SK-MES-1, SK-N-SH, SL3, SW403, Stimulus-triggered Acquisition of Pluripotency (STAP) cell or derivate SW403, T-cells, THP-1, Tumor cells, U2OS, U937, peripheral blood lymphocytes, expanded T cells, hematopoietic stem cells, or Vero cells.

### XIII. KITS

Certain aspects of the present invention also concern kits containing compositions of the disclosure or compositions to implement methods of the disclosure. In some embodiments, kits can be used to detect the presence of a cancer cell in a sample. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, a kit contains one or more polypeptides capable of binding to NT5E, including polypeptides disclosed herein. For example, a kit may comprise at least 1, 2, 3, 4, 5, 6, 7, 8, 9, 10 or more antibodies or antibody fragments (e.g., Fabs) disclosed herein for detecting NT5E, in some cases for detecting NT5E on a surface of a cancer cell. In some embodiments, a kit comprises a detection pair. In some embodiments, a kit comprises an enzyme. In some embodiments, a kit comprises a substrate for an enzyme.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. In certain aspects, negative and/or positive control nucleic acids, probes, and inhibitors are included in some kit embodiments.

Kits may further comprise instructions for use. For example, in some embodiments, a kit comprises instructions for detecting NT5E, such as NT5E on a surface of a cancer cell, in a sample.

It is contemplated that any method or composition described herein can be implemented with respect to any other method or composition described herein and that different embodiments may be combined. The claims originally filed are contemplated to cover claims that are multiply dependent on any filed claim or combination of filed claims.

### XIV. EXAMPLES

The following examples are included to demonstrate certain embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1—Development of Synthetic Human Monoclonal Antibodies that Inhibit NT5E in an ATP-Dependent Manner

A phage display strategy was designed to generate tumor selective inhibitors that exploit the high ATP concentration hallmark of the tumor microenvironment (TME). Several Fabs were identified with binding and inhibitory properties positively influenced by high ATP concentrations. Two Fabs, named 1780 and 1789, exhibiting promising characteristics on cancer cell lines were carried forward for more detailed analyses, and they were benchmarked against Medi9447 and BMS986179, two anti-NT5E IgGs currently in clinical trials.

### A. Materials and Methods

1. Cloning, Expression and Purification of hNT5E, mNT5E, and cNT5E

The ORFs corresponding to the human, murine and cyno NT5E were cloned into the baculovirus transfer vector pACGP67 to express the proteins of interest as secreted proteins using the GP64 signal peptide. All the constructs except the N-terminal domain construct of hNT5E had a N-terminal 6×His tag after the GP64 signal peptide and an Avi-tag at the C-terminus for potential biotinylation. The N-terminal domain construct of the hNT5E has a 6×His tag at the C-terminus and no Avi-tag. The corresponding baculoviruses were generated in Sf9 cells supplemented with 10% FBS by the standard co-transfection procedure using the BestBac 2.0 δ v-cath/chiA Linearized Baculovirus DNA (#91-002, Expression Systems). Final protein expression was carried out in Hi5 cells in a serum free media for 72 hrs at 28° C. Protein overexpressed in the media was purified by affinity chromatography using a HisTrap column (GE) followed by size exclusion chromatography (SEC) with a 5200 increase column (GE) on AKTA Explorer. Elution fractions collected from monodispered peaks were collected for further experiments. For protein biotinylation, protein dialyzed against 20 mM HEPES, 150 mM NaCl, 50 μM EDTA was biotinylated with E. coli biotin ligase BirA followed by SEC. BirA selectively biotinylates the single lysine residue in the Avi-tag leaving all the other lysine residues in the protein unmodified. The biotinylation efficiency was tested by a standard streptavidin pull-down assay.

### 2. Streptavidin Pull-Down Assay

The pull-down assay was performed using streptavidin (SA) magnetic beads (#Z5482, Promega) at RT and all incubation steps were for 15 min. 3 μg of target was incubated with 100 μl of SA magnetic beads with gentle mixing. Unbound protein fraction (flow-through) was collected and beads were washed three times. The extent of biotinylation was analyzed by comparing the amount of protein in the different fractions (input, flow-through, wash and beads) by SDS-PAGE.

### 3. Phage Display Selection

Selection for hNT5E was performed according to published protocols with slight modifications. All the selection campaigns were performed in buffers supplemented with 1 mM ATP and 10 μM zinc chloride. Primarily two strategies were followed:

(A) Epitope masking selection: In the first round of selection, 200 nM of biotinylated hNT5E was immobilized on 250 μl SA magnetic beads. This was followed by rigorous washing steps to remove unbound protein followed by a 5 min incubation with 5 μM D-biotin to block unoccupied SA sites on the beads to prevent nonspecific binding of the phage pool. The beads were then incubated with the phage library containing 10¹²-10¹³ virions/ml for 30 min at 4° C. with gentle shaking. The resuspended beads containing bound phages after extensive washing were used to infect freshly grown log phase E. coli XL1-Blue cells. Phages were amplified overnight in 2YT media with 50 μg/mL ampicillin and 10⁹ p.f.u./mL of M13 K07 helper phage. To increase the stringency of selection, three additional rounds of sorting were performed with decreasing target concentration in each round (2nd round: 100 nM, 3rd round: 50 nM and 4th round: 10 nM and 5 nM) using the amplified pool of virions of the preceding round as the input. Additionally from second round, 1 μM of 1557 scFv was used during the binding steps and in all the washing steps to mask the immunodominant epitope at the N-terminus of hNT5E. The epitope of the masking scFv is the same as commercial IgG Medi9447 (see Cloning, expression and purification of antibodies section for details). Notably, unlike solid capture in the first round, the 2nd-4th rounds involve solution capture that enables isolation of high affinity binders minimizing avidity effects. These rounds are carried out in a semi-automated platform using the Kingfisher instrument. From 2nd round onwards, the bound phages were eluted using 0.1 M glycine pH 2.7. This harsh elution technique often risks the enrichment of nonspecific and SA binders. In order to eliminate them, the precipitated phage pool from 2nd round onwards were negatively selected against 100 μL of SA beads. The “precleared” phage pool was then used as an input for the selection.

(B) Cell-antigen hybrid selection with epitope masking: In order to increase the probability of obtaining high affinity binders that bind to NT5E on cell surface of cancer cells, the inventors integrated both purified antigen and human breast cancer cell line MDA-MB-231 in the selection campaign. The first two rounds were performed on purified biotinylated hNT5E like Strategy (A). The third round was performed on MDA-MB-231 cells where 2×10⁶ cells were incubated with the phage pool amplified from the elution of second round at RT for 20 min with gentle shaking. The incubation step was followed by washing and elution with glycine as in other steps. Instead of 0.05% Tween 20 in the buffer, the buffer for this round was supplemented with 1% BSA to avoid cell lysis. The fourth round was performed on 20 nM purified biotinylated target. The epitope masking 1557 scFv was used in molar excess during the binding and washing steps in rounds 2-4 like in Strategy (A).

4. Cloning, Expression, and Purification of Fabs and 1557 scFv

Positive clones selected based on a phage ELISA were sequenced at DNA Sequencing Facility at The University of Chicago. Unique clones were reformatted using Infusion cloning (#638911, Takara Bio Inc.) in pRH2.2, an IPTG inducible vector for bacterial expression of Fabs.

E. coli C43 cells were transformed with sequence-verified clones of Fabs in pRH2.2. Fabs were grown in autoinduction media (Terrific Broth supplemented with 0.4% glycerol+media supplemented with 0.8% glycerol, 0.01% glucose, 0.02% lactose, 1.25 mM magnesium sulphate) with 100 μg/mL ampicillin at 37° C. for 6 h during and further grown overnight at 30° C. The Fabs were purified using affinity (protein L) followed by ion-exchange chromatography using an automated program on AKTA explorer system. Briefly, pellets were resuspended in PBS, supplemented with 1 mM PMSF, 1 μg/mL DNase I. The suspension was lysed by ultrasonication. The cell lysate was incubated at 65° C. for 30 min. Heat-treated lysate was then cleared by centrifugation, filtered through 0.22 μm filter and loaded onto a HiTrap Protein L (GE Healtchcare) column pre-equilibrated with 20 mM Tris 500 mM NaCl pH 7.5. The column was washed extensively with 20 mM Tris 500 mM NaCl pH 7.5 followed by elution of Fabs with 0.1 M acetic acid. Fractions containing protein were directly loaded onto an ion-exchange Resource S 1-ml column pre-equilibrated with 50 mM NaOAc pH 5.0. Column was washed with the equilibration buffer and Fabs were eluted with a linear gradient 0-50% of 50 mM NaOAc 2 M NaCl pH 5.0. Purified Fabs were buffer exchanged against 20 mM HEPES 150 mM NaCl pH 7.5. The quality of purified Fabs was analyzed by SDS-PAGE. His-tagged 1557 scFv was cloned in pRH2.2, overexpressed in E. coli BL21(gold) cells by IPTG induction and purified over HisTrap column followed by protein A affinity chromatography.

### 5. Cloning, Expression and Purification of Antibodies

Heavy and light chains of IgG were cloned into pSCSTa-hG1 and pSCSTa-hK expression vectors, respectively. Plasmid DNA were purified using QIAfilter plasmid maxi kit according to the manufacturer's protocol (QIAGEN). BMS986179 IgG was made in-house. Medi9447 was ordered as purified IgG from Absolute Antibodies.

Gibco ExpiFectamine 293 Reagent was used for transfection of suspension Expi293 cells with plasmid DNA according to the manufacturer's protocol (ThermoFisher Scientific). Final transfection volumes varied from 90 mL to 360 mL cell culture and a mixture of 0.5 μg pSCSTa-hG1 DNA and 0.5 μg pSCSTa-hK DNA was used per 1 mL cell culture. Following transfection, cells were grown in vented baffled flasks for 96 hrs in a 37° C. incubator with a humidified atmosphere of 8% CO2 in air on an orbital shaker rotating at 125 rpm.

Cells were centrifuged at 3000×g for 10 min and the IgG containing supernatant was poured into appropriately sized sterile disposable bottle. About 1 mL of protein A recombinant fast flow sepharose beads (GE Healthcare) per 100 mL transfection supernatant was added and incubated for 2 hrs on a rotating platform (125 rpm) at RT. Supernatant with protein A beads was poured into column/funnel combo and drained. IgG bound resin was washed with 40× the bead volume of 1×DPBS (ThermoFisher) and then eluted with 13 mL of IgG elution buffer (ThermoFisher). The eluted IgG sample was neutralized by adding 1.3 mL of neutralization buffer (1M Tris pH 8.0, ThermoFisher). Precipitates were spun down at 12000×g for 10 mins and supernatant was concentrated and 1×DPBS buffer exchanged on Amicon Ultra-15 centrifugal filters 50 kDa (Fisher). Concentrated sample was sterilized through Steriflip filter system (0.22 μm, low protein binding, Millipore). Protein concentration was determined by spectrophotometry and IgG quality was assessed by SDS-PAGE under reducing and non-reducing conditions.

### 6. ELISA Experiments

(A) Phage ELISA: All ELISA experiments were performed in a 96-well plate coated with 50 μL of 2 μg/mL neutravidin in Na2CO3 pH 9.6 and subsequently blocked by 0.5% in PBS. A single-point phage ELISA was used to rapidly screen the binding of Fabs obtained in phage format. Colonies of E. coli XL1-Blue harboring phagemids were inoculated directly into 500 μL of 2YT broth supplemented with 100 μg/ml ampicillin and M13-KO7 helper phage. The cultures were grown at 37° C. for 16-20 h in a 96-deep well block plate. Culture supernatants containing Fab phage were diluted 10-fold in ELISA buffer (20 mM HEPES, 150 mM NaCl, 0.05% Tween 20, 10 μM zinc chloride, 1 mM ATP pH 7.4) and transferred to ELISA plates pre-incubated with either 50 nM of biotinylated target protein or buffer alone control wells for 15 min at RT. ELISA plates were washed with PBST then incubated with HRP-conjugated anti-M13 mouse monoclonal antibody (#MM05T-H, SinoBiological Inc., 1:3000 dilution) for 30 min. The plates were again washed, developed with TMB substrate and quenched with 1.0 M HCl, and absorbance (Abs450) was determined. The background phage binding was monitored from control wells.

(B) Protein ELISA: Protein-based multipoint ELISA was performed to estimate the affinity of the generated Fabs and IgGs to NT5E. The binding of the Fabs and IgGs to the closed form of NT5E were determined in ELISA buffer (HBST) supplemented with 10 μM zinc chloride and 1 mM ATP while the binding of the same to open form of NT5E were determined in ELISA buffer (HBST) without any cofactors. A fixed concentration of the immobilized target (50 nM) on ELISA plate was incubated with 3-fold serial dilutions of the purified Fabs and IgGs starting from 3 μM and 1 μM respectively for 15 min. The plates were washed and were subsequently incubated with a secondary HRP-conjugated anti-protein L monoclonal antibody (#32420, ThermoFisher, 1:6000 dilution). As with phage ELISA, the plates were again washed, developed with TMB substrate and quenched with 1.0 M HCl, and absorbance (Abs450) was determined. To determine the affinity (EC50), the data were fitted with a non-linear, 4-parameter logistic curve in OriginLab.

(C) Sandwich Protein ELISA—Epitope Binning: ELISA plate was coated with 50 nM BMS986179 IgG and Medi9447 IgG overnight at 4° C. followed by blocking with 1% (w/v) BSA in HBS for two hours at RT. The plate was washed with ELISA buffer (10 mM HEPES, 150 mM NaCl, 0.05% (v/v) Tween 20, 1 mM ATP, pH 7.5). 200 nM biotinylated full-length hNT5E alone and with 1 μM 1789 Fab was added to the plate, incubated at RT with gentle shaking for 20 min and washed again with the ELISA buffer. Strep-HRP antibody (#N100, ThermoFisher, 1:20,000 dilution) was added to the wells, incubated at RT with gentle shaking for 30 min, washed, and then the signal developed with TMB substrate followed by quenching with 1 M HCl. The ELISA signal was measured at 450 nm in an Epoch Biotech Plate Reader.

### 7. Thermal Shift Assay

Thermal stability of samples was analyzed by differential scanning fluorimetry (DSF) using a real-time PCR instrument (Bio-Rad CFX384). Protein samples were mixed with 4× Sypro Orange at a final 4 μM concentration. For target/Fab complexes, 1.5-fold molar excess of Fab was used. All reactions were performed in triplicates in a 384-well plate. Thermal melting curves were monitored from 25° C. to 95° C. in steps of 0.5° C./30 s. Wavelengths of 490 and 575 nm were used for excitation and emission, respectively. The melting temperatures were inferred from the first derivative of the melting curves from the mean of triplicate runs using CFX software provided by the manufacturer.

### 8. Fluorescence Polarization

Fluorescence measurements were made in SynergyNeo microplate reader (BioTek Instruments), using a custom made dual PMT filter cube with excitation filter 310/20 nm and emission filter 420/27 nm (PN241, BioTek Instruments, Inc.). 60 ml samples contained a combination of the following components: 2 mM hNT5E, 2 mM Fab or 1 mM IgG, and 2 mM etheno-ATP. hNT5E was pre-incubated with Fab or IgG in 20 mM HEPES, 150 mM KCl pH 7.5 buffer for 10 min at 25° C. Then, etheno-ATP was added and samples were loaded into F-bottom, black 384-well microplate (Grenier). Parallel and perpendicular fluorescence intensities were measured over 90 min with 2 min intervals. For background correction, parallel and perpendicular fluorescence intensities of corresponding protein mixes lacking etheno-ATP were measured. After background subtraction, fluorescence polarization was calculated by Equation 1 below.

$$FP = \frac{{I}_{par} - {I}_{perp}}{{I}_{par} + {I}_{perp}} \times 1000 \qquad ( 1 )$$

### 9. Binding Kinetics, Single Domain Binding Experiments, and Domain Binning by SPR

All SPR experiments were performed at 25° C. using MASS-1 (Bruker) instrument. hNT5E, mNT5E and cNT5E were immobilized onto a nitrilotriacetic acid (NTA) sensor chip via His-tag. For kinetic experiments, after ligand immobilization, 2-fold serial dilutions of the Fabs were injected. After double-reference subtraction data were analyzed with BiaEvaluation (GE Healthcare) or Sierra Analyser (Bruker) software. The experiments were carried in buffers in presence and absence of 1 mM ATP to compare the kinetic parameters of the Fabs binding to closed and open form of NT5E.

In the domain binding experiments, full-length, N-terminal, and C-terminal hNT5E were immobilized as described above. Single shot injections were done of fixed 1789 Fab at 200 nM in ATP-free buffer.

In the domain binning experiments, full-length, N-terminal, and C-terminal hNT5E were immobilized as described above. Single shot injections were done of fixed 1789 Fab at 200 nM in ATP-free buffer.

### 10. Flow Cytometry Analysis of Antibody Binding to Cellular NT5E

Human MDA-MB-231 cells and murine 4T1 cells were grown to 90% confluency, detached with 5 mM EDTA for 15 min at 37° C. and then resuspended in 37° C. lx PBS (#10010031, Gibco) supplemented with 1% FBS and 1OpM ZnCl2. Separate working buffers (WB) of PBS and 1% FBS with or without fixed nucleotide concentrations were used throughout all washing steps to maintain constant condition. In 96-well plates (#3799, Costar), 0.2×10⁶ cells were added per well. The plates were spun at 500×g for 5 min and the supernatant was removed. Anti-NT5E antibodies were diluted to the desired concentration in 100 μL WB, added to the cells and the plates were incubated for 20 min at 37° C. Plates were spun and cells were washed twice with WB then spun a second time. Cells were stained with 100 μL of secondary antibody (Alexa Fluor 647 AffiniPure Goat Anti-Human IgG, F(ab′)2 fragment specific, #109-605-006, Jackson, 1:200) dilution and plates were incubated for 20 min at 37° C. Cells were washed twice with WB then fixed on ice with WB supplemented with 0.5% PFA. Fixing solution was removed by centrifugation and cells were resuspended in cold 1× PBS. Samples were analyzed by flow cytometry using CytoFLEX (Beckman Coulter) and mean fluorescence intensity (MFI) data was extracted using FlowJo software. Resulting binding data was plotted using OriginLab. For binding of 1789 Fab with different fixed nucleotide concentrations, data generated on different days were normalized to the Medi9447 IgG positive control.

### 11. Antibody Inhibition of NT5E Expressed on Cells

Phosphate production by cellular NT5E was analyzed using the EnzChek Phosphate Assay Kit (E6646, ThermoFisher Scientific). Cell assay buffer consisted of 10 mM HEPES, 10 mM glucose, 125 mM NaCl, 2 mM MgCl₂, 1 mM KCl, and 50 μM POM-1 pH 7.2. NT5E-expressing MDA-MB-231 (human) and 4T1 (murine) cells were seeded in a 96-well plate (#353072, Falcon) at 20,000 cells per well in DMEM medium (#10013CV, Corning) supplemented with 1% PS and 10% FBS. After 16 hrs at 37° C. 5% CO₂, the cells were washed twice with warm cell assay buffer then returned to the incubator for 10 min with 50 μL fresh buffer. Cells were then treated with antibodies±500 μM_{f} ATP for 30 min at 37° C. 5% CO₂. Reaction was initiated by the addition of 200 μM_{f} AMP and allowed to proceed for 30 min (<20% product formation). Appropriate cell background controls were included in quadruplicate for each data set: Cells alone, cells with 500 μM_{f} ATP. Plate was spun at 200×g for 5 min and supernatant was transferred to a UV flat bottom plate (#8404, ThermoFisher) prepared with EnzChek Phosphate Assay kit reagents including an 8-point phosphate standard made with cell assay buffer. No significant background phosphate was detected for cell assay buffer with ATP. The final absorbance at 360 nm was measured with SynergyNeo microplate reader (BioTek Instruments). Data was extracted and the mean values for each condition±ATP were normalized to the relative % activity with the respective±ATP controls, see Equation 2 and 3. Normalized data therefore represented the inhibition by antibody alone and not a combination of antibody and ATP (competitive inhibitor of NT5E).

$$Relative \% {Activity}_{NoATP} = 100 - \frac{{Cells}_{experimental} - {Cells}_{AMP}}{{Cells}_{alone} - {Cells}_{AMP}} \times 100 \qquad ( 2 )$$

$$Relative \% {Active}_{ATP} = 100 - \frac{{Cells}_{experimental + ATP} - {Cells}_{AMP + ATP}}{{Cells}_{alone + ATP} - {Cells}_{AMP + ATP}} \times 100 \qquad ( 3 )$$

To determine the half maximal inhibitory concentration (IC₅₀), the data were fitted with a non-linear, 4-parameter logistic curve in OriginLab.

### 12. Affinity Maturation of 1789

To increase the affinity/inhibitory properties of parental 1789, and importantly the murine cross-reactivity, an affinity maturation campaign was undertaken. CDRs L3, H1, H2 and H3 were diversified to generate six sub-libraries. The randomization strategies were only focused on “tailored” and “soft” randomization techniques to minimize epitope drift and loss of inhibitory properties of the parent Ab. The six phage libraries used for selection were as follows (a) L3-tailored, (b) H1-tailored, (c) H2-tailored, (d) H3-soft, (e) combo-tailored, and (f) combo-soft. These libraries were used as individual inputs in the selection campaign.

In parallel to the regular selection, another campaign was performed in which the F417A/F500A mutant of the hNT5E, denoted as hNT5E_{FF}, was used as the soluble competitor. This mutant of hNT5E was designed to impair the binding of ATP to the nucleotide-binding pocket. Thus, in addition to supplementing 1 mM ATP and 10 μM zinc chloride in standard selection buffers (HBST+0.5% BSA), using this mutant as the soluble competitor should significantly increase binders specific for ATP-bound (closed) conformation by eliminating conformation-agnostic binders in the wash steps. Four rounds of selection were performed; the first two rounds and the 4th round were performed on hNT5E while the 3rd round was done on mNT5E to increase affinity (cross-reactivity) of the binders to the murine protein. The regular and the competitive strategies resulted in similar outcomes with some variations among the libraries. Enrichment ratio, which is a measure of the specific clones over background binders, was the best for the H2-tailored sub-library. 96 clones from each selection were subjected to single-point phage ELISA. In the single-point phage ELISA, the binding of the clones to hNT5E (WT), hNT5E_{FF}, and mNT5E (WT) were tested. Clones with high binding to the hNT5E (WT), mNT5E (WT) and low/no binding to the hNT5E_{FF} were chosen for further characterization. Unique clones, verified by DNA sequencing, were reformatted in Fab format in pRH2.2 vector, expressed in E. coli C43+(pro) cells by autoinduction, purified by protein L and resource S chromatography, and characterized for affinity and inhibitory properties by SPR and cell-based inhibition assays on human and murine cancer cell lines as described for parental 1789.

### B. Results

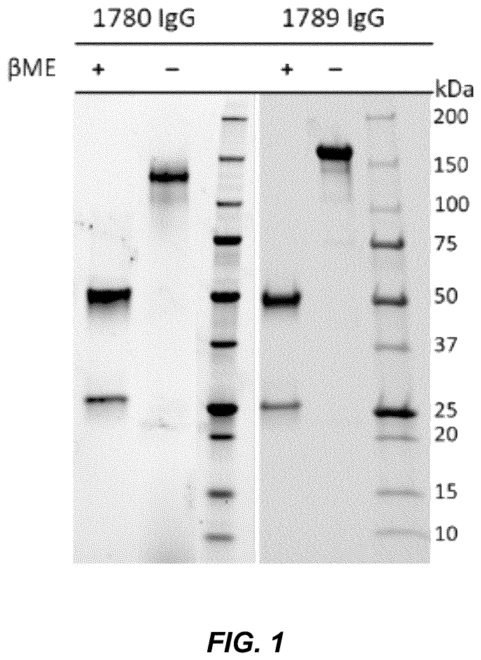

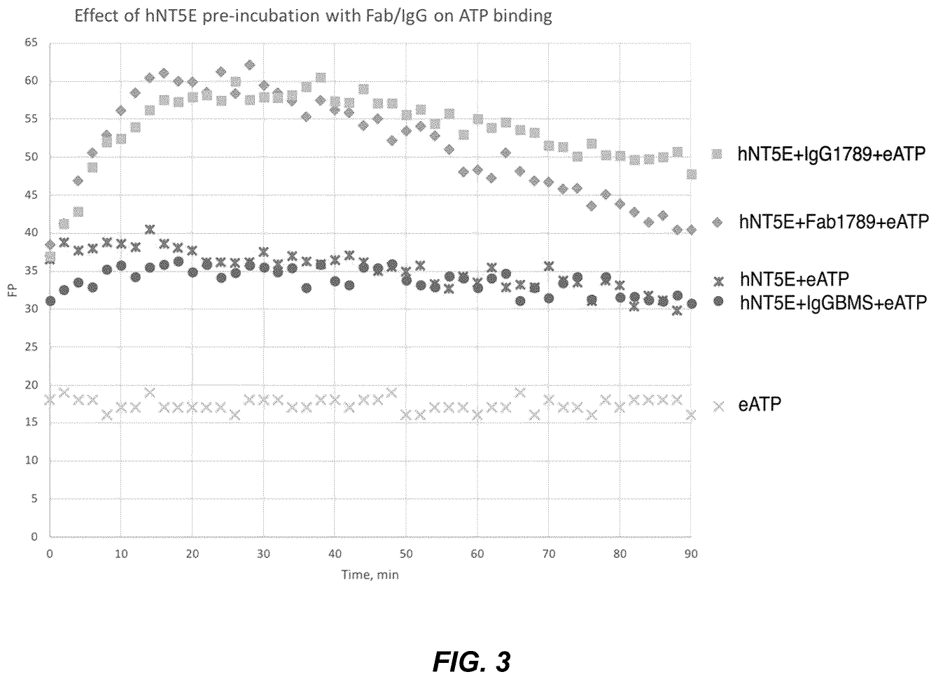

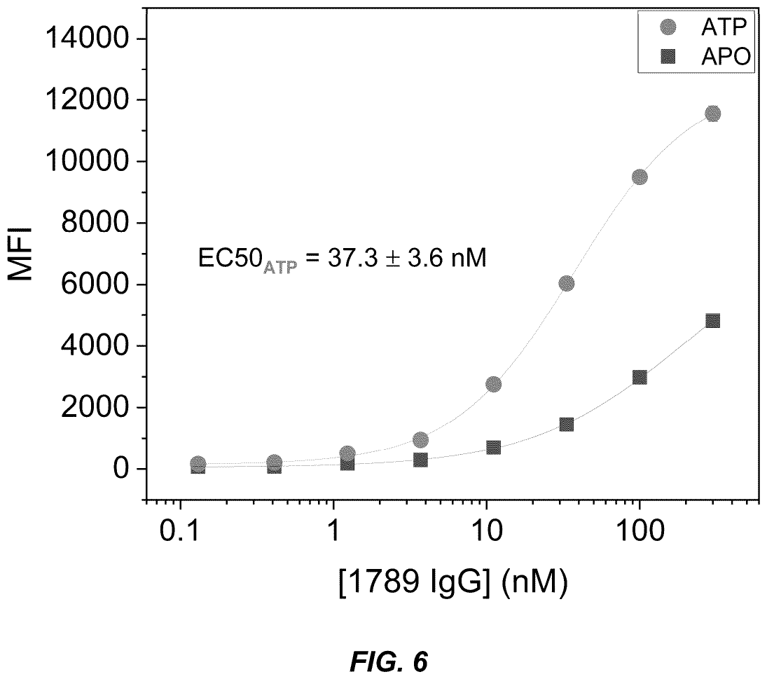

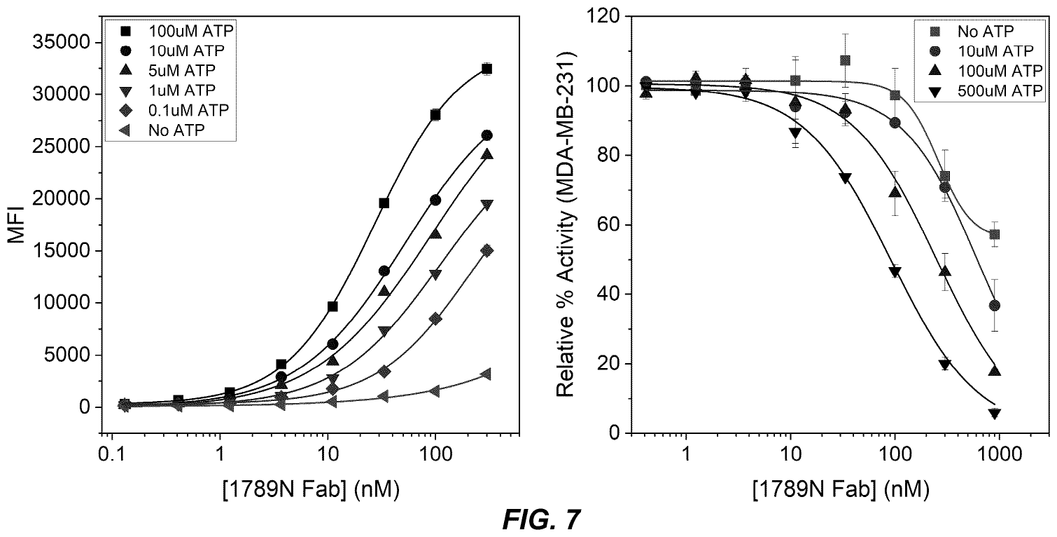

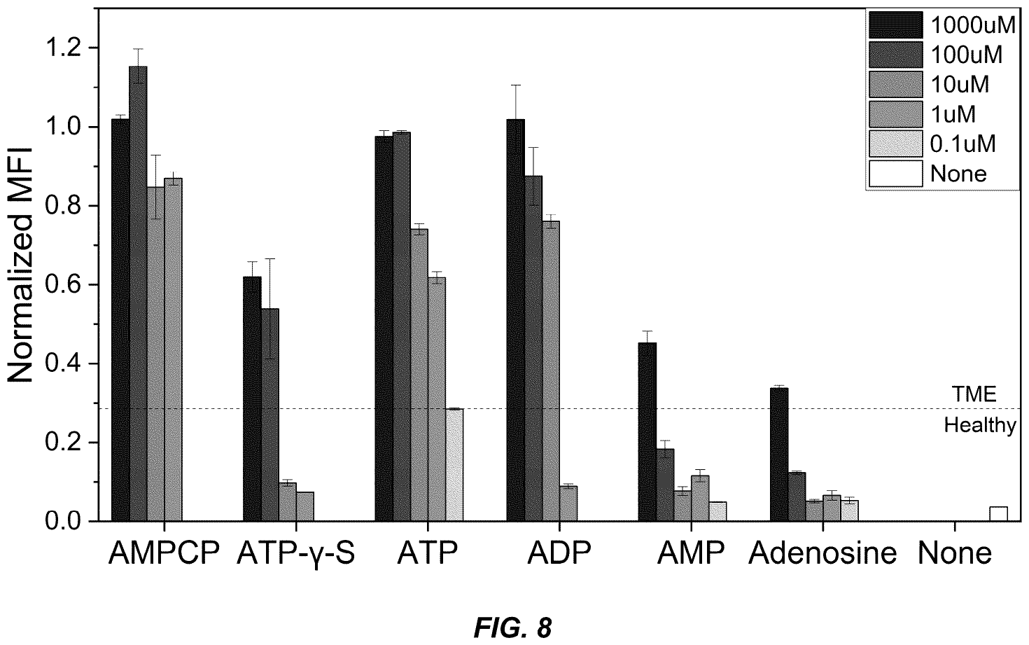

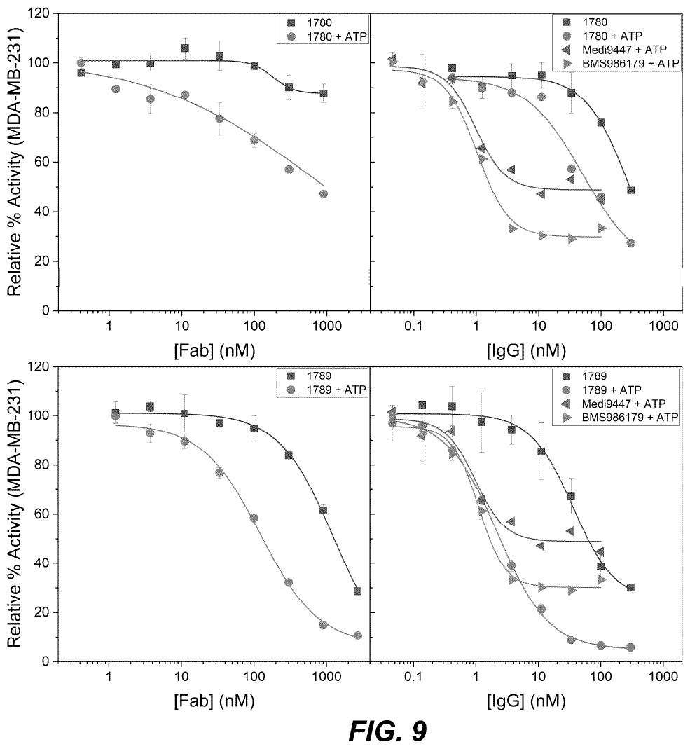

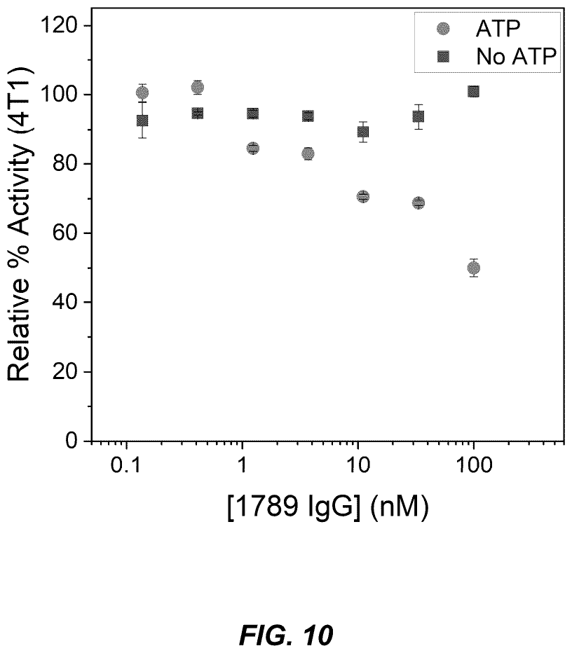

Results from characterization of 1780 and 1789 Fabs and IgGs are shown in FIGS. 1-11.

### 1. Summary

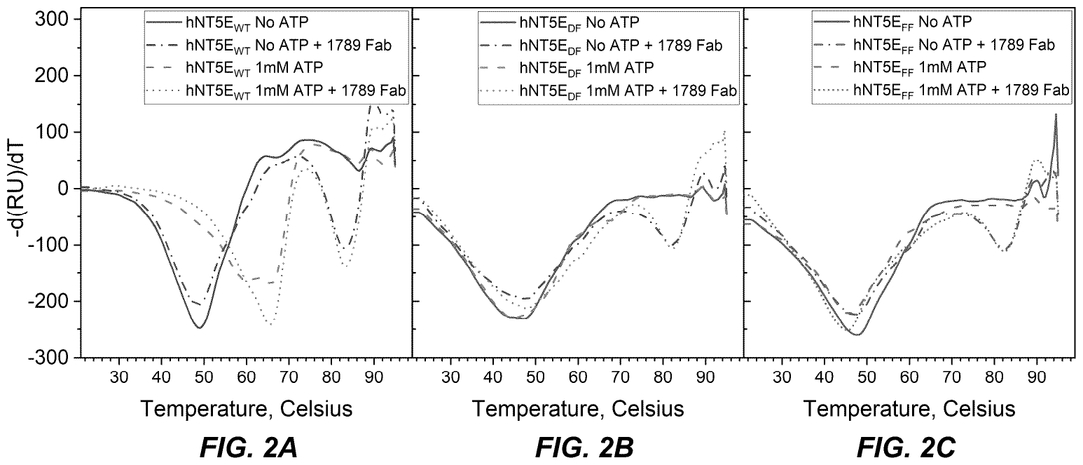

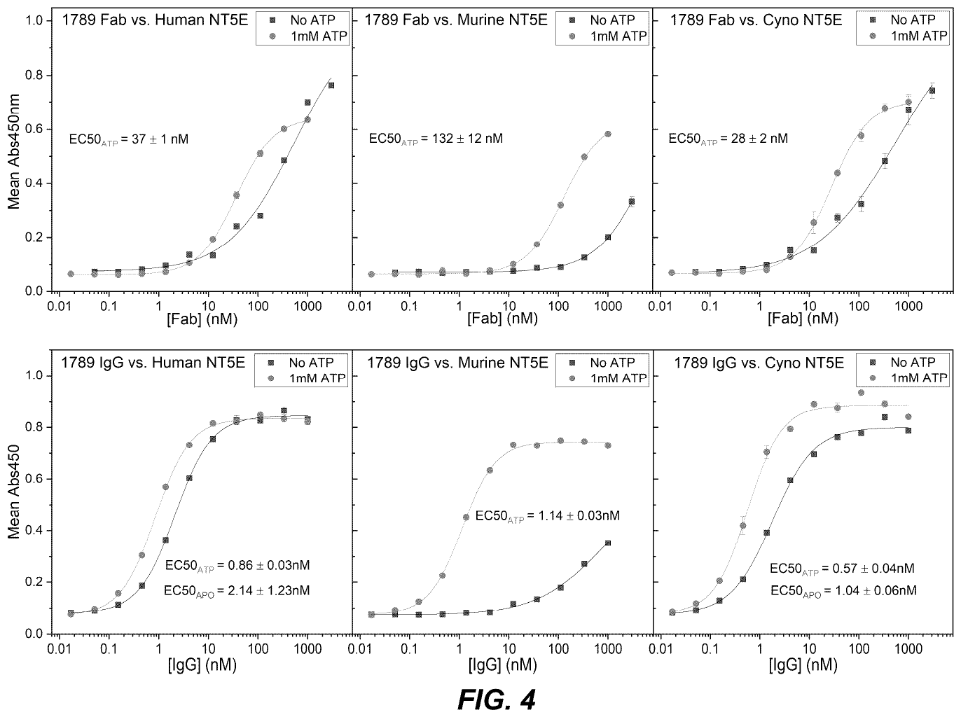

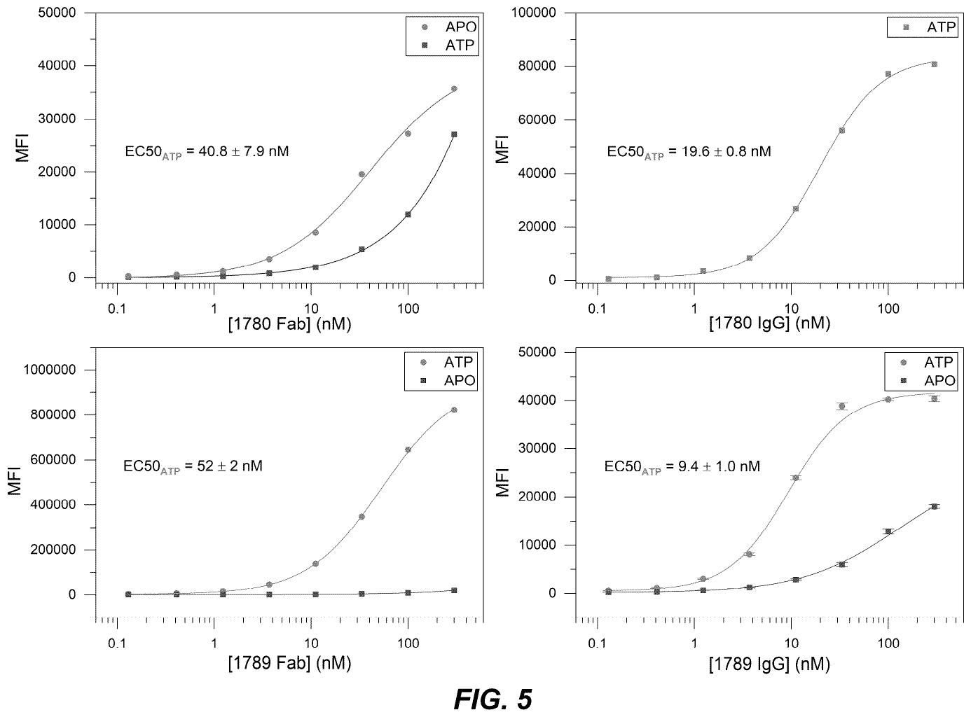

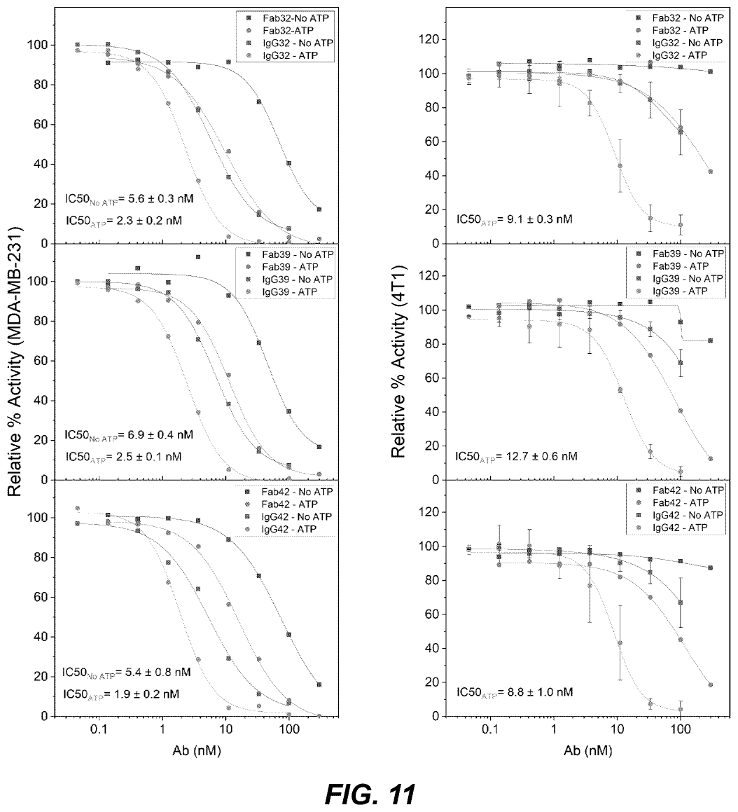

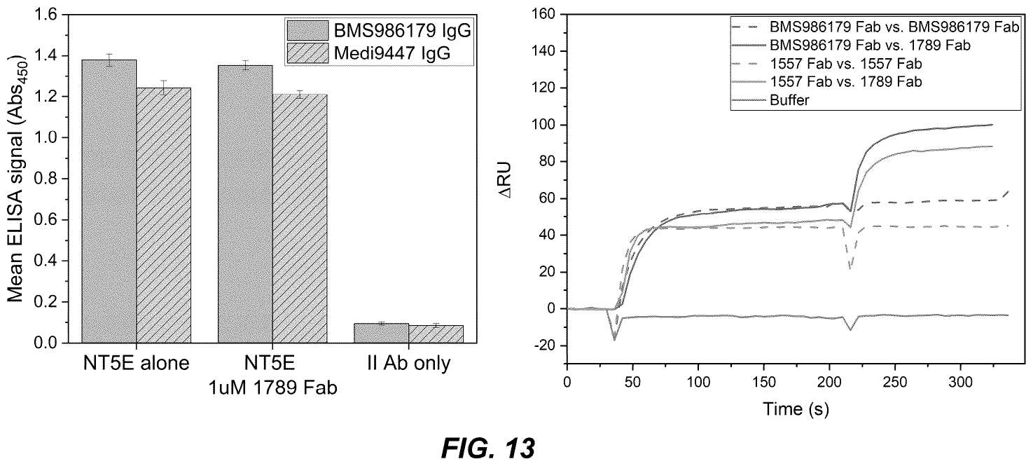

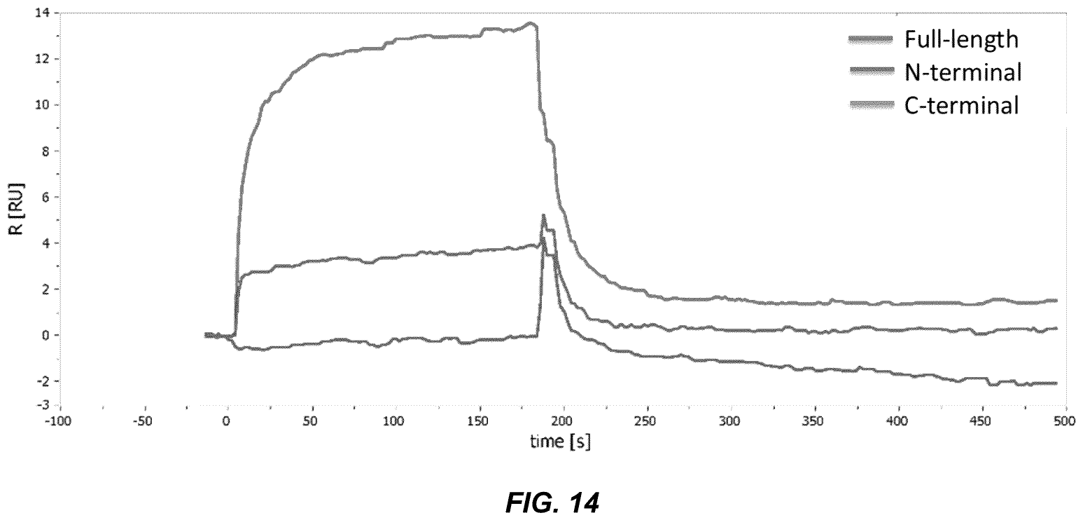

1. ATP-dependent binding and inhibition on cells (Table 7; FIGS. 5-9)
2. Cross-reactivity between human, murine, and cynolomogus NT5E (Tables 2 and 5; FIG. 4)
3. Epitope is unique from the benchmark antibodies Medi9447 and BMS986179 (FIGS. 2 and 13-14)
4. Affinity maturation of 1789 generated variants with significantly improved inhibitory properties on murine 4T1 cancer cells (FIG. 11)

TABLE 4
Selection conditions of lead antibodies.

| | | | | Lead | |
|---|---|---|---|---|---|
| Sel. ID | Antigen | Cell | Library | IgGs | Conditions |
| 98ZB2-2 | hNT5E(in-house) | N/A | E | 1780 | Epitope masking on Ag |
| | | | | | with 1557scFv and |
| | | | | | 1 mM ATP. |
| 102SH3-2 | hNT5E(in-house) | MDA-MB-231 | E | 1789 | Cell-Ag hybrid with |
| | | | | | epitope masking and |
| | | | | | 1 mM ATP. |

TABLE 5
In vitro cross-reactivity analysis by surface plasmon resonance. SPR binding
constants for 1789 Fab against human, murine, and cyno NT5E antigens in
ATP-free and 1 mM ATP supplemented buffer. Note: due to complex
kinetic analysis of bivalent interactions, data of IgG against dimeric
NT5E is not included. SPR data could not be obtained for 1780 Fab.

| | | 1789 Fab | |
|---|---|---|---|
| Species | Binding Constants | No ATP | 1 mM ATP |
| Human NT5E | kₒₙ M s⁻¹ | 5.7 × 10⁴ | 1.7 × 10⁵ |
| | k_{off} s⁻¹ | 4.4 × 10⁻² | 7.3 × 10⁻³ |
| | K_{D} M | 7.6 × 10⁻⁷ | 4.2 × 10⁻⁸ |
| Murine NT5E | kₒₙ M s⁻¹ | no binding | 8.5 × 10⁴ |
| | k_{off} s⁻¹ | no binding | 1.4 × 10⁻² |
| | K_{D} M | no binding | 1.6 × 10⁻⁷ |
| | kₒₙ M s⁻¹ | 1.4 × 10⁵ | 1.6 × 10⁵ |
| Cyno NT5E | k_{off} s⁻¹ | 2.0 × 10⁻² | 5.1 × 10⁻³ |
| | K_{D} M | 1.5 × 10⁻⁷ | 3.2 × 10⁻⁸ |

TABLE 6
Summary of T_{M} for hNT5E_{WT} hNT5E_{DF}, and hNT5E by differential scanning
fluorimetry. Thermal stability shifts were measured for hNT5E_{WT} and substrate binding
knock-out variants, hNT5E_{DF} and hNT5E_{FF}, to assess the dependence of 1789 Fab-mediated
stabilization of hNT5E in the presence of ATP. Melting temperatures (T_{M}) are for NT5E only
and are represented by the mean of three replicates. The TM for 1789 Fab is 83° C. with or
without ATP.

| | | No ATP | 1 mM ATP |
|---|---|---|---|
| hNT5E_{W T} | — | 49° C. | 59° C. and 64° C. |
| hNT5E_{W T} | 1789 Fab | 48° C. | 66° C. |
| hNT5E_{DF} | — | 44° C. | 46° C. |
| hNT5E_{DF} | 1789 Fab | 48° C. | 46° C. |
| hNT5E_{FF} | — | 47° C. | 47° C. |
| hNT5E_{FF} | 1789 Fab | 47° C. | 46° C. |

TABLE 7
Binding and enzyme inhibition characteristics of NT5E antibodies against human
NT5E-expressing MDA-MB-231 cells and mouse NT5E-expressing 4T1 cells. MDA-MB-
231 and 4T1 EC50 data acquired from flow cytometry analysis of antibody binding to cell lines
in PBS alone or in PBS with 1 mM ATP. IC₅₀ data acquired from end-point phosphate analysis
of antibody-mediated inhibition of NT5E on MDA-MB-231 cells in the absence or presence of
500 μM ATP. Binding to murine 4T1 cells with single [Fab] (200 nM) or [IgG] (100 nM) are
denoted as weak, moderate, or strong.

| | | Human Cell Line MDA-MB-231 | | Murine Cell Line 4T1 |
|---|---|---|---|---|
| | | EC50 | IC50 | EC50 |
| Antibody | Condition | nM | nM | nM |
| 1789 Fab | −ATP | no fit | no fit | weak |
| | +ATP | 53.0 ± 2.2 | 122.2 ± 12.3 | weak |
| 1789 IgG | −ATP | 123.9 ± 42.5 | no fit | no fit |
| | +ATP | 9.4 ± 1.0 | 2.2 ± 0.2 | 37 ± 4 |
| 1780 Fab | −ATP | no fit | no fit | none |
| | +ATP | 37.0 ± 5.8 | no fit | none |
| 1780 IgG | −ATP | no data | no fit | weak |
| | +ATP | 28 ± 2 | 50.5 ± 25.6 | moderate |

TABLE 8
In vitro cross-reactivity analysis of affinity matured 1789 variants by SPR. SPR
binding constants for affinity matured 1789 Fab variants against human and murine NT5E
antigens in ATP-free and 1 mM ATP supplemented buffer. * Data generated poor fits.

| | Binding | Human | | Murine | |
|---|---|---|---|---|---|
| Fab ID | Constants | No ATP | 1 mM ATP | No ATP | 1 mM ATP |
| AM24 | kₒₙ M s⁻¹ | 1.2 × 10⁵ | 1.7 × 10⁵ | 1.0 × 10⁵ | 1.5 × 10⁵ |
| | k_{off} s⁻¹ | 6.0 × 10⁻³ | 3.4 × 10⁻³ | 2.0 × 10⁻² | 7.0 × 10⁻³ |
| | K_{D} M | 4.9 × 10⁻⁸ | 2.0 × 10⁻⁸ | 1.9 × 10⁻⁷ | 4.7 × 10⁻⁸ |
| AM28 | kₒₙ M s⁻¹ | 1.9 × 10⁵ | 9.4 × 10⁵ | 4.3 × 10⁴ | 1.8 × 10⁵ |
| | k_{off} s⁻¹ | 8.6 × 10⁻³ | 5.5 × 10⁻³ | 4.7 × 10⁻³ | 1.4 × 10⁻² |
| | K_{D} M | 4.6 × 10⁻⁸ | 2.3 × 10⁻⁸ | 1.1 × 10⁻⁷ * | 7.8 × 10⁻⁸ |
| AM29 | kₒₙ M s⁻¹ | 1.2 × 10⁵ | 1.4 × 10⁵ | 3.8 × 10⁴ | 1.0 × 10⁵ |
| | k_{off} s⁻¹ | 1.0 × 10⁻² | 5.1 × 10⁻³ | 7.0 × 10⁻³ | 9.0 × 10⁻³ |
| | K_{D} M | 8.7 × 10⁻⁸ | 3.7 × 10⁻⁸ | 1.9 × 10⁻⁷ | 8.7 × 10⁻⁸ |
| AM31 | kₒₙ M s⁻¹ | 2.3 × 10⁵ | 4.2 × 10⁵ | 3.4 × 10⁵ | 2.6 × 10⁵ |
| | k_{off} s⁻¹ | 9.7 × 10⁻³ | 4.2 × 10⁻³ | 1.3 × 10⁻² | 4.0 × 10⁻³ |
| | K_{D} M | 4.2 × 10⁻⁸ | 1.0 × 10⁻⁸ | 1.0 × 10⁻⁷ * | 1.5 × 10⁻⁸ |
| AM32 | kₒₙ M s⁻¹ | 1.1 × 10⁵ | 2.7 × 10⁵ | 7.8 × 10⁴ | 1.9 × 10⁵ |
| | k_{off} s⁻¹ | 9.1 × 10⁻³ | 4.2 × 10⁻³ | 1.1 × 10⁻² | 2.8 × 10⁻³ |
| | K_{D} M | 8.6 × 10⁻⁸ | 1.5 × 10⁻⁸ | 1.5 × 10⁻⁷ | 1.5 × 10⁻⁸ |
| AM38 | kₒₙ M s⁻¹ | 1.9 × 10⁵ | 3.7 × 10⁵ | 1.0 × 10⁵ | 1.9 × 10⁵ |
| | k_{off} s⁻¹ | 9.0 × 10⁻³ | 3.7 × 10⁻³ | 1.2 × 10⁻² | 3.0 × 10⁻³ |
| | K_{D} M | 4.8 × 10⁻⁸ | 1.0 × 10⁻⁸ | 1.2 × 10⁻⁷ | 1.6 × 10⁻⁸ |
| AM39 | kₒₙ M s⁻¹ | 2.8 × 10⁵ | 4.9 × 10⁵ | 1.4 × 10⁵ | 2.9 × 10⁵ |
| | k_{off} s⁻¹ | 9.9 × 10⁻³ | 3.9 × 10⁻³ | 1.3 × 10⁻² | 3.5 × 10⁻³ |
| | K_{D} M | 3.5 × 10⁻⁸ | 7.9 × 10⁻⁹ | 9.9 × 10⁻⁸ * | 1.2 × 10⁻⁸ |
| AM42 | kₒₙ M s⁻¹ | 1.8 × 10⁵ | 3.2 × 10⁵ | 1.9 × 10⁵ | 2.5 × 10⁵ |
| | k_{off} s⁻¹ | 6.7 × 10⁻³ | 3.6 × 10⁻³ | 1.2 × 10⁻² | 2.9 × 10⁻³ |
| | K_{D} M | 3.8 × 10⁻⁸ | 1.1 × 10⁻⁸ | 6.3 × 10⁻⁸ | 1.2 × 10⁻⁸ |
| AM45 | kₒₙ M s⁻¹ | 2.3 × 10⁵ | 2.7 × 10⁵ | 5.8 × 10⁴ | 1.7 × 10⁵ |
| | k_{off} s⁻¹ | 1.4 × 10⁻² | 4.5 × 10⁻³ | 1.3 × 10⁻² | 5.8 × 10⁻³ |
| | K_{D} M | 6.0 × 10⁻⁸ | 1.7 × 10⁻⁸ | 2.2 × 10⁻⁷ | 3.5 × 10⁻⁸ |

### Example 2—Radiolabeling of AM-39 and 1798

To conjugate DOTA to AM39, the antibody (2000 mL at a concentration of 1.50 mg/mL) was dispersed in 100 mL of 1.0M sodium bicarbonate buffer (pH 9.0). 1,4,7,10-Tetraazacyclododecane-1,4,7,10-tetraacetic acid mono-N-hydroxysuccinimide ester (20 mM DMSO, 30 molar excess) was added to the antibody solution dropwise while mixing vigorously. The final concentration of DMSO was kept below 2% (v/v) to avoid any precipitation. The reaction was incubated for 90 min at 37° C. The reaction mixture was purified with a PD-10 column using an ammonium acetate mobile phase (0.2 M sodium acetate, pH 6.0). The DOTA-4A06 solution was aliquoted and stored at −20° C. until time of use. Similarly, 1798 was conjugated to DOTA.

A solution of ¹⁷⁷LuCl₃ (8mCi; 10 ml) was diluted with 190 mL 0.2M ammonium acetate. 2 mg in 2.5 mL of DOTA-AM39 (pH=6) were added into the reaction vial. The reaction was carried out at 37° C. for 1 h. The reaction progress was monitored by ITLC using a 20 mM citric acid (pH 4.9-5.1) mobile phase. The radiolableing efficiency was 75%. The product was purified with a PD10 column. The decay corrected radiochemical yield was consistently>95%. Similarly, DOTA-1798 was labeled with Lu-177.

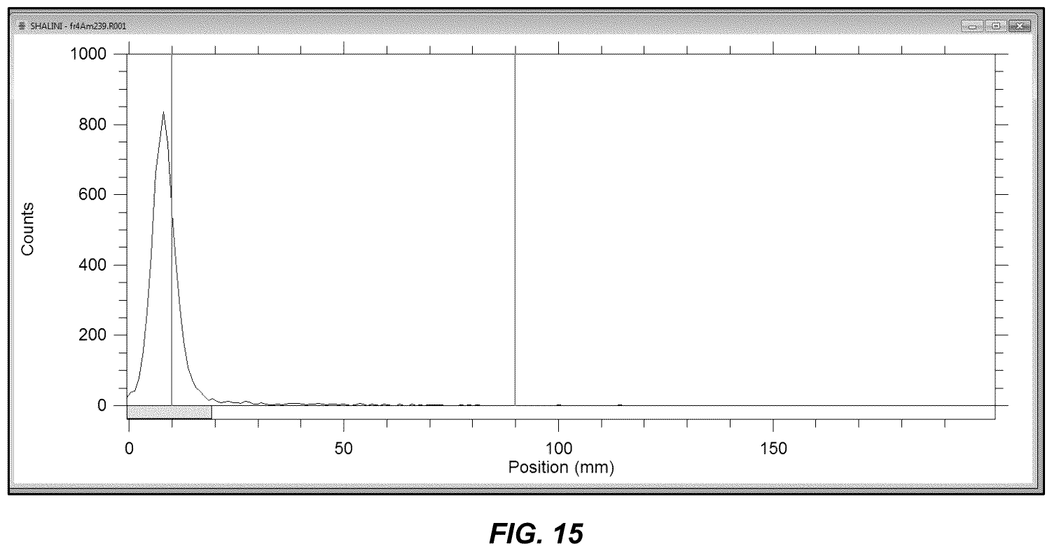

¹⁷⁷Lu radiolabeling of AM-39: In a 1.5 mL reaction vial, 1 mCi (2 μL) 177Lu was diluted with 200 μL 1M CH₃COONH₄ (pH=5.5). A solution of DOTA-AM39 (250 μg) in 100 μL CH₃COONH₄ (pH=5.5) was added to the reaction vial. The reaction mixture was incubated at 37° C. for 1 hour. The progress of the reaction was monitored using a radio-iTLC. After 1 hour, the crude reaction mixture was purified using a G25 desalting column and eluted with PBS (pH=7.4) in 100 μL fractions with a radio chemical yield of ˜95%. The pure fractions with maximum activity were pooled and formulated in PBS (pH=7.4) for animal studies. Complex purity>99% (FIG. 15).

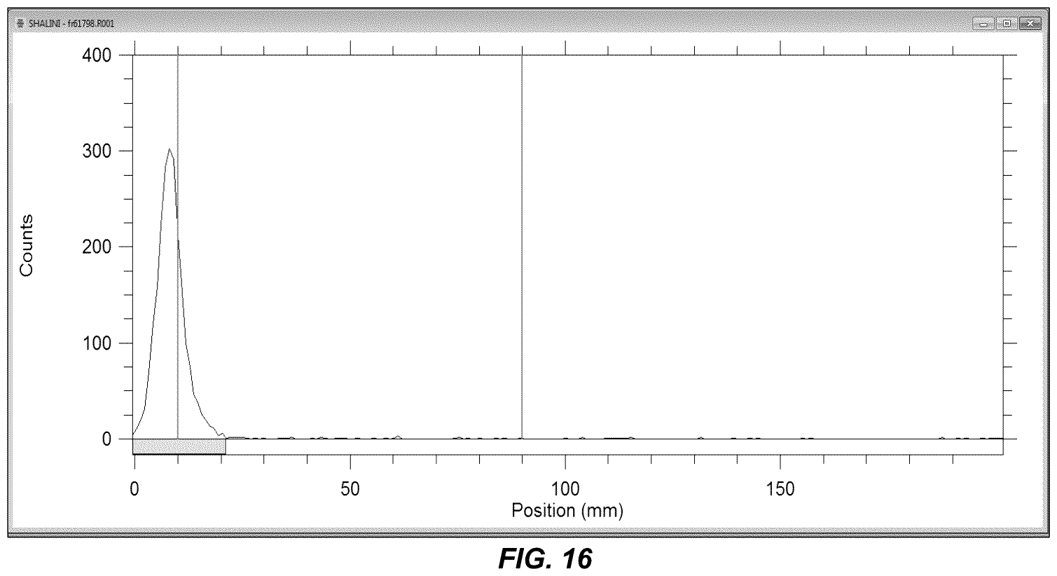

¹⁷⁷Lu radiolabeling of 1798: In a 1.5 mL reaction vial, 1 mCi (2 μL) 177Lu was diluted with 200 μL 1M CH₃COONH₄ (pH=5.5). A solution of DOTA-1798 (300 μg) in 100 μL CH₃COONH₄ (pH=5.5) was added to the reaction vial. The reaction mixture was incubated at 37° C. for 2 hours. The progress of the reaction was monitored using a radio-iTLC. After 2 hours, the crude reaction mixture was purified using a G25 desalting column and eluted with PBS (pH=7.4) in 100 μL fractions with a radio chemical yield of ˜45%. The pure fractions with maximum activity were pooled and formulated in PBS (pH=7.4) for animal studies. Complex purity>99% (FIG. 16).

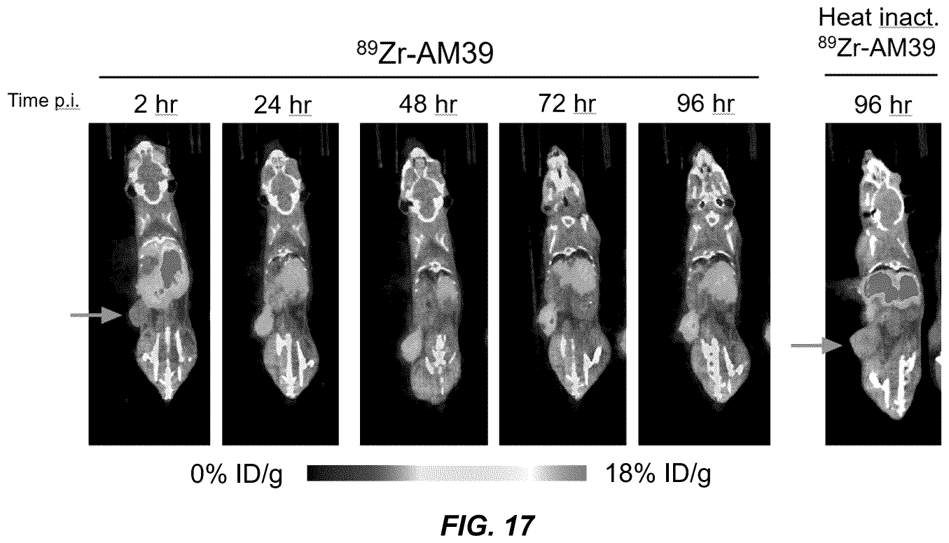

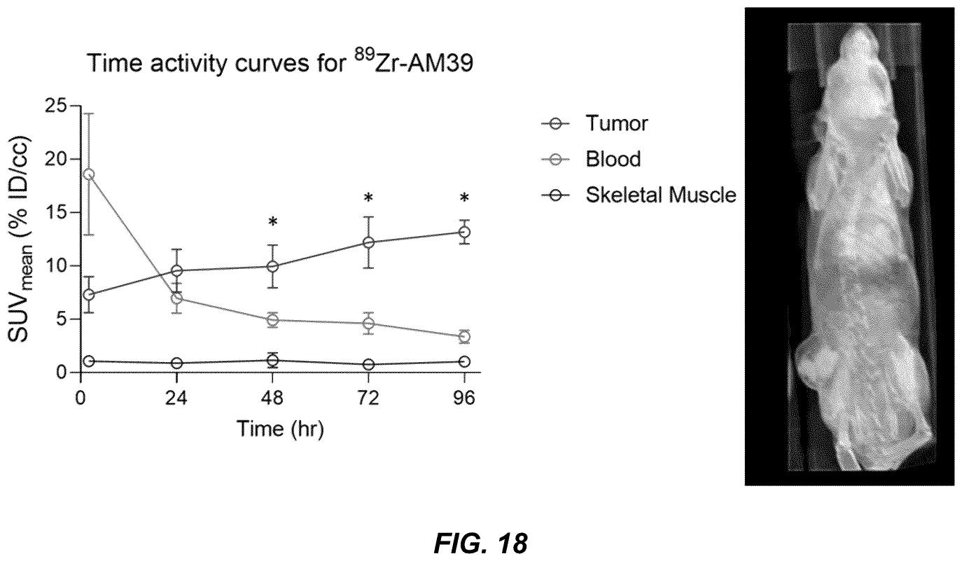

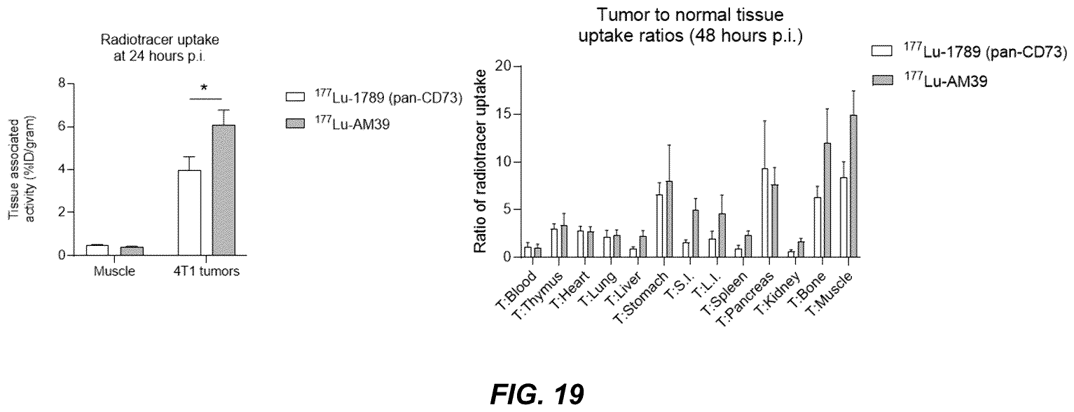

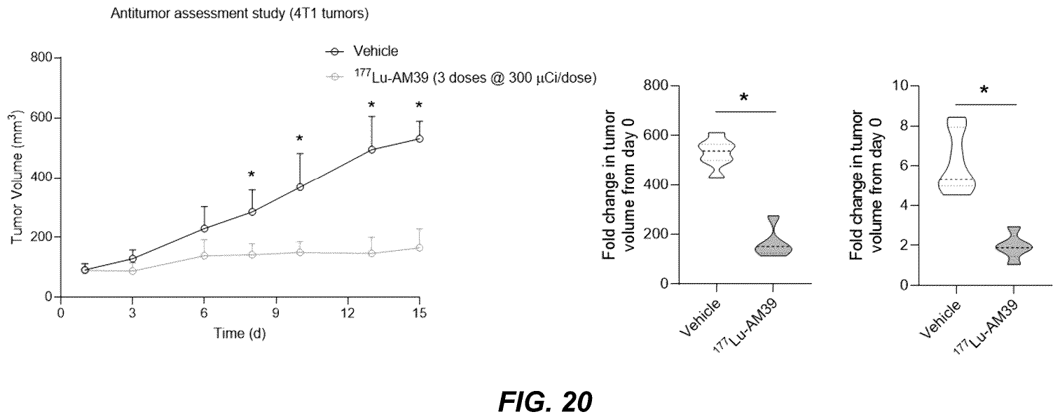

As shown in FIG. 17, ⁸⁹Zr-AM39 (radiolabeled NT5E antibody) specifically localizes to 4T1 tumors in vivo. ⁸⁹Zr-AM39 IgG1 was injected intravenously into female Balb/c mice bearing subcutaneous 4T1 tumors. Mice (n=4) we imaged longitudinally on PET/CT out to 120 hours post injection. Representative coronal PET/CT images show the localization of the radiotracer to the tumor and the persistence of binding out to 96 hours post injection (red arrow). Little binding in normal tissue is observed, with the exception of the liver (accumulation due to clearance). A control arm was injected with ⁸⁹Zr-AM39 IgG that was heated to 98° C. to denature the IgG prior to injection. No uptake was observed in the tumor, which suggests the tumoral accumulation of intact ⁸⁹Zr-AM39 IgG is driven by specific antigen binding. FIG. 18 shows the time activity profiles of ⁸⁹Zr-AM39 distribution through the tumor bearing mouse. Tumoral uptake occurred rapidly, and continuously rose out to 96 hours post injection. The tumoral uptake was significantly higher than blood or muscle by 48 hours post injection. At right is shown a maximum intensity projection acquired at 96 hours post injection. The MIP shows no detectable signal outside of the tumor and liver. Shown in FIG. 19 are the biodistribution studies acquired at 24 hours post injection in female balb/c mice bearing 4T1 tumors (left). This demonstrates that ¹⁷⁷Lu-AM39 has higher tumoral uptake than ¹⁷⁷Lu-1789, an equipotent anti-CD73 IgG with an ATP independent binding mode (termed “pan-CD73” as the antibody does not distinguish between CD73 in tumors versus normal tissue, *P<0.01). FIG. 19 also shows tumor to background ratios for each antibody at 24 hours post injection (right). AM39 tumor to background ratio was significantly higher than 1789 for liver, small intestine (SI), large intestine (LI), kidney, bone, muscle. FIG. 20 shows tumor volume data from an antitumor assessment study using ¹⁷⁷Lu-AM39 (right). Female balb/c mice bearing subcutaneous 4T1 tumors (n=8) were injected with ¹⁷⁷Lu-AM39 intravenously on day 0 and day 6 of the study. Mice received ˜300 μCi per injection. Tumor growth was significantly inhibited by drug treatment starting from day 8. FIG. 20 also shows the tumor volume and normalized tumor volume values on day 15 (FIG. 20—right). ¹⁷⁷Lu-AM39 significantly inhibited tumor growth (*P<0.01).

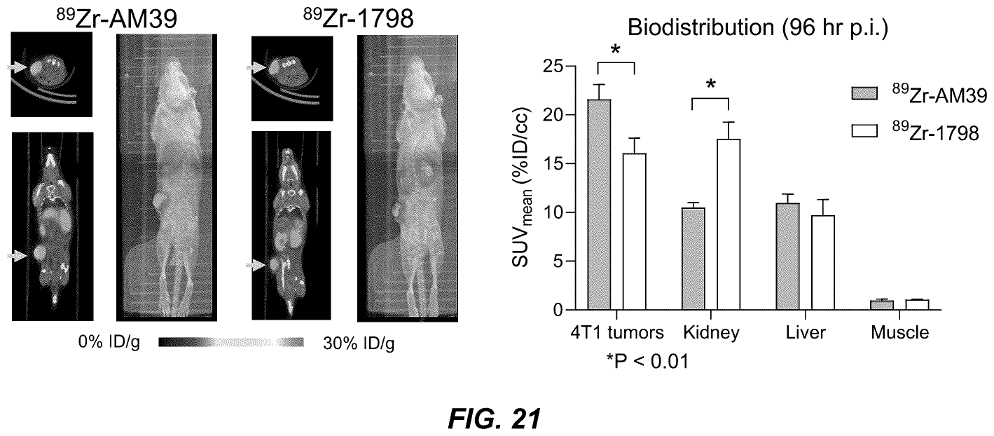

FIG. 21 shows the biodistribution of two radiotracers in mice bearing subcutaneous 4T1 tumors. Mice received the indicated radiotracer at ˜300 uCi/mouse and were imaged at 96 hours post injection. The tumor uptake of ⁸⁹Zr-AM39 was higher than ⁸⁹Zr-1798, while NT5E rich organs like the kidney were lower. Radiotracer levels were equivalent in organs of clearance (e.g. the liver) and in skeletal muscle, a tissue compartment representing background.

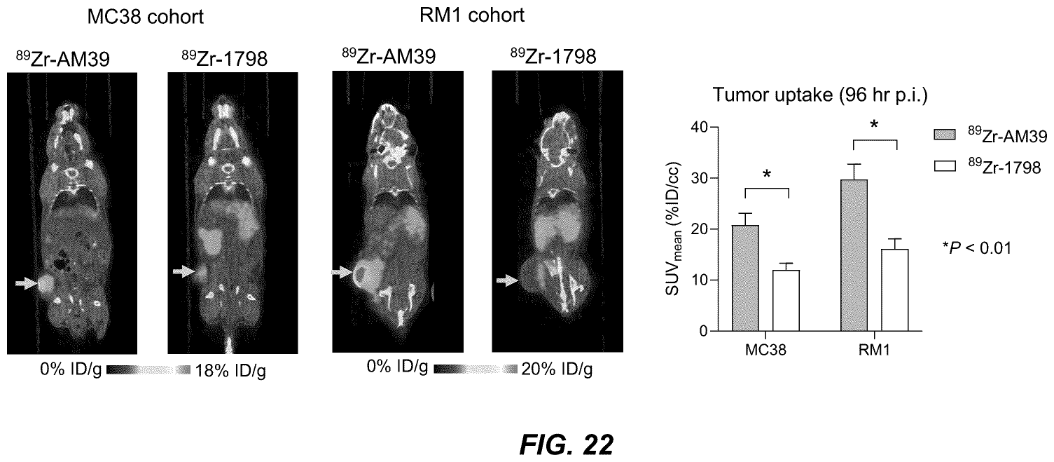

FIG. 22 shows tumor uptake of two radiotracers in mice bearing subcutaneous MC38 (colorectal) or RM1 (prostate) tumors. Mice received ˜300 uCi of the indicated radiotracer. AM39 uptake in the tumor was significantly higher than 1798 (FIG. 22, right panel).

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, and those cited elsewhere herein, to the extent that they provide exemplary procedural or other details supplementary to those set forth herein, are specifically incorporated herein by reference.
1. Knapp, K., Zebisch, M., Pippel, J., El-Tayeb, A., Muller, C. E., and Strater, N. (2012) Crystal Structure of the Human Ecto-5-Nucleotidase (CD73): Insights into the Regulation of Purinergic Signaling. Structure 20, 2161-2173.
2. Heuts, D. P. H. M., Weissenborn, M. J., Olkhov, R. V., Shaw, A. M., Gummadova, J., Levy, C., and Scrutton, N. S. (2012) Crystal Structure of a Soluble Form of HumanCD73 with Ecto-5′-Nucleotidase Activity. ChemBioChem 13.
3. Leone, R., and Emens, L. (2018) Targeting adenosine for cancer immunotherapy. J. Immunotherapy Cancer 6.
4. Beavis, P. A., Stagg, J., Darcy, P. K., and Smyth, M. J. (2012) CD73: a potent suppressor of antitumor immune responses. Trends in Immunology 33, 231-237.
5. Zhang, B. (2012) CD73 promotes tumor growth and metastasis. OncoImmunology 1, 67-70, PMID: 22720214.
6. Antonioli, L., Pacher, P., Vizi, S., and Hasko, G. (2013) CD39 and CD73 in immunity and inammation. Trends in Molecular Medicine 19, 355-367.
7. Gao, Z.-w., Dong, K., and Zhang, H.-z. (2014) The Roles of CD73 in Cancer. BioMed Research International 9 pages.
8. Rahimova, R., Fontanel, S., Lionne, C., Jordheim, L. P., Peyrottes, S., and Chaloin, L. (2018) Identification of allosteric inhibitors of the ecto-5′-nucleotidase (CD73) targeting the dimer interface. PLOS Computational Biology 14, 1-23.
9. Di Virgilio, F., and Adinolfi, E. (2017) Extracellular purines, purinergic receptors and tumor growth. Oncogene 36, 293-303.
10. Fellouse, F. A., Esaki, K., Birtalan, S., Raptis, D., Cancasci, V. J., Koide, A., Jhurani, P., Vasser, M., Wiesmann, C., Kossiakoff, A. A., Koide, S., and Sidhu, S. S. (2007) High throughput Generation of Synthetic Antibodies from Highly Functional Minimalist Phagedisplayed Libraries. Journal of Molecular Biology 373, 924-940.
11. Paduch, M., Koide, A., Uysal, S., Rizk, S. S., Koide, S., and Kossiakoff, A. A. (2013) Generating conformation-specific synthetic antibodies to trap proteins in selected functional states. Methods 60, 3-14, In Vitro Protein Selection.
12. Miller, K. R., Koide, A., Leung, B., Fitzsimmons, J., Yoder, B., Yuan, H., Jay, M., Sidhu, S. S., Koide, S., and Collins, E. J. (2012) T Cell Receptor-Like Recognition of Tumor In Vivo by Synthetic Antibody Fragment. PLOS ONE 7, 1-14.
13. Niesen, F., Berglund, H., and Vedadi, M. (2007) The use of differential scanning uorimetry to detect ligand interactions that promote protein stability. Nat Protoc 2, 2212-2221.

What is claimed is:

1. An antibody or antigen binding fragment comprising a heavy chain variable region and a light chain variable region, wherein the heavy chain variable region comprises a HCDR1, HCDR2, and HCDR3 from a heavy chain variable region of Table 1 and wherein the light chain variable region comprises a LCDR1, LCDR2, and LCDR3 from a light chain variable region of Table 1.

2. An antibody or antigen binding fragment comprising a heavy chain variable region and a light chain variable region, wherein the heavy chain variable region comprises a HCDR1, HCDR2, and HCDR3 having at least 80% sequence identity with a HCDR1, HCDR2, and HCDR3 from a heavy chain variable region of Table 1 and wherein the light chain variable region comprises a LCDR1, LCDR2, and LCDR3 having at least 80% sequence identity with a LCDR1, LCDR2, and LCDR3 from a light chain variable region of Table 1.

3. The antibody of claim 1 or 2, wherein the heavy chain variable region and light chain variable region of Table 1 are from the same antibody clone of Table 1.

4. An antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:1-3, respectively, and wherein the light chain variable region comprises a LCDR3 comprising the amino acid sequence of SEQ ID NO:6.

5. An antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:1-3, respectively, and wherein the light chain variable region comprises a LCDR1, LCDR2, and LCDR3 comprising the amino acid sequence of SEQ ID NOs:4-6, respectively.

6. An antibody or antigen binding fragment comprising a heavy chain variable region and a light chain variable region, wherein the heavy chain variable region comprises a HCDR1, HCDR2, and HCDR3 having at least 80% sequence identity to SEQ ID NOs:1-3, respectively, and wherein the light chain variable region comprises a LCDR1, LCDR2, and LCDR3 having at least 80% sequence identity to SEQ ID NOs:4-6, respectively.

7. The antibody or antigen binding fragment of any of claims 4-6, wherein the heavy chain variable region comprises an amino acid sequence with at least 80% sequence identity to SEQ ID NO:7 and/or the light chain variable region comprises an amino acid sequence with at least 80% sequence identity to SEQ ID NO:8.

8. The antibody or antigen binding fragment of any of claims 4-7, wherein the heavy chain variable region comprises the amino acid sequence of SEQ ID NO:7 and/or the light chain variable region comprises the amino acid sequence of SEQ ID NO:8.

9. The antibody or antigen binding fragment of any one of claims 4-8, wherein the antibody or antigen binding fragment comprises an amino acid sequence with at least 70% sequence identity to one of SEQ ID NOs:1-10 and/or an amino acid sequence with 1 substitution relative to SEQ ID NOs:1-10.

10. The antibody of any one of claims 4-9, wherein the antibody comprises a heavy chain and a light chain and wherein the heavy chain comprises an amino acid sequence with at least 70% sequence identity to SEQ ID NO:9 and the light chain comprises an amino acid sequence with at least 70% sequence identity to SEQ ID NO:10.

11. An antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:11-13, respectively, and wherein the light chain variable region comprises a LCDR3 comprising the amino acid sequence of SEQ ID NO:16.

12. An antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:11-13, respectively, and wherein the light chain variable region comprises a LCDR1, LCDR2, and LCDR3 comprising the amino acid sequence of SEQ ID NOs:14-16, respectively.

13. An antibody or antigen binding fragment comprising a heavy chain variable region and a light chain variable region, wherein the heavy chain variable region comprises a HCDR1, HCDR2, and HCDR3 having at least 80% sequence identity to SEQ ID NOs:11-13, respectively, and wherein the light chain variable region comprises a LCDR1, LCDR2, and LCDR3 having at least 80% sequence identity to SEQ ID NOs:14-16, respectively.

14. The antibody or antigen binding fragment of any of claims 11-13, wherein the heavy chain variable region comprises an amino acid sequence with at least 80% sequence identity to SEQ ID NO:17 and/or the light chain variable region comprises an amino acid sequence with at least 80% sequence identity to SEQ ID NO:18.

15. The antibody or antigen binding fragment of any of claims 11-14, wherein the heavy chain variable region comprises the amino acid sequence of SEQ ID NO:17 and/or the light chain variable region comprises the amino acid sequence of SEQ ID NO:18.

16. The antibody or antigen binding fragment of any one of claims 11-15, wherein the antibody or antigen binding fragment comprises an amino acid sequence with at least 70% sequence identity to one of SEQ ID NOs:11-20 and/or an amino acid sequence with 1 substitution relative to SEQ ID NOs:11-20.

17. The antibody of any one of claims 11-16, wherein the antibody comprises a heavy chain and a light chain and wherein the heavy chain comprises an amino acid sequence with at least 70% sequence identity to SEQ ID NO:19 and the light chain comprises an amino acid sequence with at least 70% sequence identity to SEQ ID NO:20.

18. An antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:21-23, respectively, and wherein the light chain variable region comprises a LCDR3 comprising the amino acid sequence of SEQ ID NO:26.

19. An antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:21-23, respectively, and wherein the light chain variable region comprises a LCDR1, LCDR2, and LCDR3 comprising the amino acid sequence of SEQ ID NOs:24-26, respectively.

20. An antibody or antigen binding fragment comprising a heavy chain variable region and a light chain variable region, wherein the heavy chain variable region comprises a HCDR1, HCDR2, and HCDR3 having at least 80% sequence identity to SEQ ID NOs:21-23, respectively, and wherein the light chain variable region comprises a LCDR1, LCDR2, and LCDR3 having at least 80% sequence identity to SEQ ID NOs:24-26, respectively.

21. The antibody or antigen binding fragment of any of claims 18-20, wherein the heavy chain variable region comprises an amino acid sequence with at least 80% sequence identity to SEQ ID NO:27 and/or the light chain variable region comprises an amino acid sequence with at least 80% sequence identity to SEQ ID NO:28.

22. The antibody or antigen binding fragment of any of claims 18-21, wherein the heavy chain variable region comprises the amino acid sequence of SEQ ID NO:27 and/or the light chain variable region comprises the amino acid sequence of SEQ ID NO:28.

23. The antibody or antigen binding fragment of any one of claims 18-22, wherein the antibody or antigen binding fragment comprises an amino acid sequence with at least 70% sequence identity to one of SEQ ID NOs:21-30 and/or an amino acid sequence with 1 substitution relative to SEQ ID NOs:21-30.

24. The antibody of any one of claims 18-23, wherein the antibody comprises a heavy chain and a light chain and wherein the heavy chain comprises an amino acid sequence with at least 70% sequence identity to SEQ ID NO:29 and the light chain comprises an amino acid sequence with at least 70% sequence identity to SEQ ID NO:30.

25. An antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:31-33, respectively, and wherein the light chain variable region comprises a LCDR3 comprising the amino acid sequence of SEQ ID NO:36.

26. An antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:31-33, respectively, and wherein the light chain variable region comprises a LCDR1, LCDR2, and LCDR3 comprising the amino acid sequence of SEQ ID NOs:34-36, respectively.

27. An antibody or antigen binding fragment comprising a heavy chain variable region and a light chain variable region, wherein the heavy chain variable region comprises a HCDR1, HCDR2, and HCDR3 having at least 80% sequence identity to SEQ ID NOs:31-33, respectively, and wherein the light chain variable region comprises a LCDR1, LCDR2, and LCDR3 having at least 80% sequence identity to SEQ ID NOs:34-36, respectively.

28. The antibody or antigen binding fragment of any of claims 25-27, wherein the heavy chain variable region comprises an amino acid sequence with at least 80% sequence identity to SEQ ID NO:37 and/or the light chain variable region comprises an amino acid sequence with at least 80% sequence identity to SEQ ID NO:38.

29. The antibody or antigen binding fragment of any of claims 25-28, wherein the heavy chain variable region comprises the amino acid sequence of SEQ ID NO:37 and/or the light chain variable region comprises the amino acid sequence of SEQ ID NO:38.

30. The antibody or antigen binding fragment of any one of claims 25-29, wherein the antibody or antigen binding fragment comprises an amino acid sequence with at least 70% sequence identity to one of SEQ ID NOs:31-40 and/or an amino acid sequence with 1 substitution relative to SEQ ID NOs:31-40.

31. The antibody of any one of claims 25-30, wherein the antibody comprises a heavy chain and a light chain and wherein the heavy chain comprises an amino acid sequence with at least 70% sequence identity to SEQ ID NO:39 and the light chain comprises an amino acid sequence with at least 70% sequence identity to SEQ ID NO:40.

32. An antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:41-43, respectively, and wherein the light chain variable region comprises a LCDR3 comprising the amino acid sequence of SEQ ID NO:46.

33. An antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:41-43, respectively, and wherein the light chain variable region comprises a LCDR1, LCDR2, and LCDR3 comprising the amino acid sequence of SEQ ID NOs:44-46, respectively.

34. An antibody or antigen binding fragment comprising a heavy chain variable region and a light chain variable region, wherein the heavy chain variable region comprises a HCDR1, HCDR2, and HCDR3 having at least 80% sequence identity to SEQ ID NOs:41-43, respectively, and wherein the light chain variable region comprises a LCDR1, LCDR2, and LCDR3 having at least 80% sequence identity to SEQ ID NOs:44-46, respectively.

35. The antibody or antigen binding fragment of any of claims 32-34, wherein the heavy chain variable region comprises an amino acid sequence with at least 80% sequence identity to SEQ ID NO:47 and/or the light chain variable region comprises an amino acid sequence with at least 80% sequence identity to SEQ ID NO:48.

36. The antibody or antigen binding fragment of any of claims 32-35, wherein the heavy chain variable region comprises the amino acid sequence of SEQ ID NO:47 and/or the light chain variable region comprises the amino acid sequence of SEQ ID NO:48.

37. The antibody or antigen binding fragment of any one of claims 32-36, wherein the antibody or antigen binding fragment comprises an amino acid sequence with at least 70% sequence identity to one of SEQ ID NOs:41-50 and/or an amino acid sequence with 1 substitution relative to SEQ ID NOs:41-50.

38. The antibody of any one of claims 32-37, wherein the antibody comprises a heavy chain and a light chain and wherein the heavy chain comprises an amino acid sequence with at least 70% sequence identity to SEQ ID NO:49 and the light chain comprises an amino acid sequence with at least 70% sequence identity to SEQ ID NO:50.

39. An antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:51-53, respectively, and wherein the light chain variable region comprises a LCDR3 comprising the amino acid sequence of SEQ ID NO:56.

40. An antibody or antigen binding fragment comprising a heavy chain variable region and a light chain variable region, wherein the heavy chain variable region comprises a HCDR1, HCDR2, and HCDR3 comprising the amino acid sequence of SEQ ID NOs:51-53, respectively, and wherein the light chain variable region comprises a LCDR1, LCDR2, and LCDR3 comprising the amino acid sequence of SEQ ID NOs:54-56, respectively.

41. An antibody or antigen binding fragment comprising a heavy chain variable region and a light chain variable region, wherein the heavy chain variable region comprises a HCDR1, HCDR2, and HCDR3 having at least 80% sequence identity to SEQ ID NOs:51-53, respectively, and wherein the light chain variable region comprises a LCDR1, LCDR2, and LCDR3 having at least 80% sequence identity to SEQ ID NOs:54-56, respectively.

42. The antibody or antigen binding fragment of any of claims 39-41, wherein the heavy chain variable region comprises an amino acid sequence with at least 80% sequence identity to SEQ ID NO:57 and/or the light chain variable region comprises an amino acid sequence with at least 80% sequence identity to SEQ ID NO:58.

43. The antibody or antigen binding fragment of any of claims 39-42, wherein the heavy chain variable region comprises the amino acid sequence of SEQ ID NO:57 and/or the light chain variable region comprises the amino acid sequence of SEQ ID NO:58.

44. The antibody or antigen binding fragment of any one of claims 39-43, wherein the antibody or antigen binding fragment comprises an amino acid sequence with at least 70% sequence identity to one of SEQ ID NOs:51-60 and/or an amino acid sequence with 1 substitution relative to SEQ ID NOs:51-60.

45. The antibody of any one of claims 39-44, wherein the antibody comprises a heavy chain and a light chain and wherein the heavy chain comprises an amino acid sequence with at least 70% sequence identity to SEQ ID NO:59 and the light chain comprises an amino acid sequence with at least 70% sequence identity to SEQ ID NO:60.

46. The antibody of any one of claims 1-45, wherein the antibody is human, chimeric, or humanized.

47. The antibody or antigen-binding fragment of any one of claims 1-46, wherein the antibody or antigen binding fragment binds NT5E with a K_{D} of about 10⁻⁶ M to about 10⁻¹² M.

48. The antibody or antigen-binding fragment of any one of claims 1-47, wherein the antibody or antigen binding fragment binds NT5E with a K_{D} of less than 20 nM.

49. The antibody or antigen-binding fragment of any one of claims 1-48, wherein the antibody or antigen binding fragment binds NT5E with a K_{D} of less than 1 nM.

50. The antibody or antigen-binding fragment of any one of claims 1-49, wherein the antibody or antigen binding fragment binds NT5E with a higher affinity in the presence of ATP than in the absence of ATP.

51. The antibody or antigen-binding fragment of any one of claims 1-50, wherein the antibody or antigen binding fragment binds NT5E with at least a 3-fold higher affinity in the presence of ATP than in the absence of ATP.

52. The antibody or antigen-binding fragment of any one of claims 1-51, wherein the antibody or antigen binding fragment binds NT5E with at least a 5-fold higher affinity in the presence of ATP than in the absence of ATP.

53. The antibody or antigen-binding fragment of any one of claims 1-52, wherein the antibody or antigen binding fragment binds NT5E with at least a 10-fold higher affinity in the presence of ATP than in the absence of ATP.

54. The antibody or antigen binding fragment of any one of claims 1-53, wherein the antibody is a human antibody, humanized antibody, recombinant antibody, chimeric antibody, an antibody derivative, a veneered antibody, a diabody, a monoclonal antibody, a single domain antibody, or a single chain antibody.

55. The antigen binding fragment of any one of claims 1-54, wherein the antigen binding fragment is a single chain variable fragment (scFv), F(ab′)₂, Fab′, Fab, Fv, or rIgG.

56. A polypeptide comprising the antigen binding fragment of any one of claims 1-55.

57. A composition comprising the antibody or antigen binding fragment of any one of claims 1-55.

58. The composition of claim 57, wherein the composition comprises a pharmaceutical excipient.

59. The composition of claim 57 or 58, wherein the composition comprises at least two antibodies or antigen binding fragments.

60. One or more nucleic acids encoding the antibody or antigen binding fragment of any one of claims 1-55 or the polypeptide of claim 56.

61. A nucleic acid encoding a antibody heavy chain variable region, wherein the nucleic acid has at least 70% sequence identity to one of SEQ ID NOs:71-76.

62. The nucleic acid of claim 61, wherein the nucleic acid has at least 90% sequence identity to one of SEQ ID NOs:71-76.

63. The nucleic acid of claim 61, wherein the nucleic acid comprises one of SEQ ID NOs: 71-76.

64. A nucleic acid encoding an antibody light chain variable region, wherein the nucleic acid has at least 70% sequence identity to one of SEQ ID NOs:78-83.

65. The nucleic acid of claim 64, wherein the nucleic acid has at least 90% sequence identity to one of SEQ ID NOs:78-83.

66. The nucleic acid of claim 64, wherein the nucleic acid comprises one of SEQ ID NOs:78-83.

67. A vector comprising the nucleic acid of any one of claims 61-66.

68. A host cell comprising the nucleic acid of any one of claims 61-66 or the vector of claim 67.

69. The host cell of claim 68, wherein the host cell is an immune cell.

70. The host cell of claim 69, wherein the host cell is a B cell.

71. The host cell of claim 69, wherein the host cell is an NK cell.

72. The host cell of claim 69, wherein the host cell is a T cell.

73. A method of a making a cell comprising transferring the nucleic acid(s) of any one of claims 61-66 or the vector of claim 67 into a cell.

74. The method of claim 73, wherein the method further comprises culturing the cell under conditions that allow for expression of a polypeptide from the nucleic acid.

75. The method of claim 74, wherein the method further comprising isolating the expressed polypeptide.

76. The method of any one of claims 73-75, wherein the cell is immune cell.

77. The method cell of claim 76, wherein the cell is a B cell.

78. The method cell of claim 76, wherein the cell is an NK cell.

79. The method cell of claim 76, wherein the cell is a T cell.

80. A method for treating or preventing cancer in a subject, the method comprising administering to the subject the antibody or antigen binding fragment of any one of claims 1-55, the polypeptide of claim 56, or the host cell of any of claims 68-72.

81. The method of claim 80, wherein the subject is a human subject.

82. The method of claim 80 or 81, wherein the subject has one or more symptoms of cancer.

83. The method of claim 80 or 81, wherein the subject does not have any symptoms of cancer.

84. The method of any one of claims 80-83, wherein the subject has been diagnosed with cancer.

85. The method of any one of claims 80-83, wherein the subject has not been diagnosed with cancer.

86. The method of any one of claims 80-85, wherein the subject has been previously treated for cancer.

87. The method of any one of claims 80-86, wherein the subject is administered an additional therapy.

88. The method of claim 87, wherein the additional therapy comprises radiotherapy, chemotherapy, or immunotherapy.

89. A method for evaluating a sample from a subject, the method comprising contacting a biological sample from the subject, or extract thereof, with at least one antibody, antigen binding fragment, or polypeptide of any one of claims 1-56.

90. The method of claim 89, wherein the at least one antibody, antigen binding fragment, or polypeptide is operatively linked to a detectable label.

91. The method of claim 89 or 90, wherein the method further comprises incubating the antibody, antigen binding fragment, or polypeptide under conditions that allow for the binding of the antibody, antigen binding fragment, or polypeptide to antigens in the biological sample or extract thereof.

92. The method of any one of claims 89-91, wherein the method further comprises detecting the binding of an antigen to the antibody, antigen binding fragment, or polypeptide.

93. The method of any one of claims 89-92, wherein the method further comprises contacting the biological sample with at least one capture antibody, antigen, or polypeptide.

94. The method of claim 93, wherein the at least one capture antibody, antigen binding fragment, or polypeptide comprises at least one antibody of claims 1-55.

95. The method of claim 93 or 94, wherein the capture antibody is linked to a solid support.

96. The method of any one of claims 89-95, wherein the biological sample comprises a tissue sample or a blood sample.

97. A method for treating a subject for cancer, the method comprising administering to the subject an effective amount of an antibody comprising:
a heavy chain variable region comprising:
a HCDR1 comprising the amino acid sequence of SEQ ID NO:1;
a HCDR2 comprising the amino acid sequence of SEQ ID NO:2; and
a HCDR3 comprising the amino acid sequence of SEQ ID NO:3; and
a light chain variable region comprising:
a LCDR1 comprising the amino acid sequence of SEQ ID NO:4;
a LCDR2 comprising the amino acid sequence of SEQ ID NO:5; and
a LCDR3 comprising the amino acid sequence of SEQ ID NO:6.

98. A method for treating a subject for cancer, the method comprising administering to the subject an effective amount of an antibody comprising:
a heavy chain variable region comprising:
a HCDR1 comprising the amino acid sequence of SEQ ID NO:11;
a HCDR2 comprising the amino acid sequence of SEQ ID NO:12; and
a HCDR3 comprising the amino acid sequence of SEQ ID NO:13; and
a light chain variable region comprising:
a LCDR1 comprising the amino acid sequence of SEQ ID NO:14;
a LCDR2 comprising the amino acid sequence of SEQ ID NO:15; and
a LCDR3 comprising the amino acid sequence of SEQ ID NO:16.

99. A method for treating a subject for cancer, the method comprising administering to the subject an effective amount of an antibody comprising:
a heavy chain variable region comprising:
a HCDR1 comprising the amino acid sequence of SEQ ID NO:21;
a HCDR2 comprising the amino acid sequence of SEQ ID NO:22; and
a HCDR3 comprising the amino acid sequence of SEQ ID NO:23; and
a light chain variable region comprising:
a LCDR1 comprising the amino acid sequence of SEQ ID NO:24;
a LCDR2 comprising the amino acid sequence of SEQ ID NO:25; and
a LCDR3 comprising the amino acid sequence of SEQ ID NO:26.

100. A method for treating a subject for cancer, the method comprising administering to the subject an effective amount of an antibody comprising:
a heavy chain variable region comprising:
a HCDR1 comprising the amino acid sequence of SEQ ID NO:31;
a HCDR2 comprising the amino acid sequence of SEQ ID NO:32; and
a HCDR3 comprising the amino acid sequence of SEQ ID NO:33; and
a light chain variable region comprising:
a LCDR1 comprising the amino acid sequence of SEQ ID NO:34;
a LCDR2 comprising the amino acid sequence of SEQ ID NO:35; and
a LCDR3 comprising the amino acid sequence of SEQ ID NO:36.

101. A method for treating a subject for cancer, the method comprising administering to the subject an effective amount of an antibody comprising:
a heavy chain variable region comprising:
a HCDR1 comprising the amino acid sequence of SEQ ID NO:41;
a HCDR2 comprising the amino acid sequence of SEQ ID NO:42; and
a HCDR3 comprising the amino acid sequence of SEQ ID NO:43; and
a light chain variable region comprising:
a LCDR1 comprising the amino acid sequence of SEQ ID NO:44;
a LCDR2 comprising the amino acid sequence of SEQ ID NO:45; and
a LCDR3 comprising the amino acid sequence of SEQ ID NO:46.

102. A method for treating a subject for cancer, the method comprising administering to the subject an effective amount of an antibody comprising:
a heavy chain variable region comprising:
a HCDR1 comprising the amino acid sequence of SEQ ID NO:51;
a HCDR2 comprising the amino acid sequence of SEQ ID NO:52; and
a HCDR3 comprising the amino acid sequence of SEQ ID NO:53; and
a light chain variable region comprising:
a LCDR1 comprising the amino acid sequence of SEQ ID NO:54;
a LCDR2 comprising the amino acid sequence of SEQ ID NO:55; and
a LCDR3 comprising the amino acid sequence of SEQ ID NO:56.


SEQUENCE LISTING (65 sequences)

SEQ ID NO: 1 - protein, 7 aa, synthetic construct
NFYSYSI

SEQ ID NO: 2 - protein, 10 aa, synthetic construct
SISSSSGYTY

SEQ ID NO: 3 - protein, 18 aa, synthetic construct
YSWWGWHMEGWWGYMNAF

SEQ ID NO: 4 - protein, 5 aa, synthetic construct
SVSSA

SEQ ID NO: 5 - protein, 7 aa, synthetic construct
SASSLYS

SEQ ID NO: 6 - protein, 6 aa, synthetic construct
AHPKPI

SEQ ID NO: 7 - protein, 127 aa, synthetic construct
EVQLVESGGGLVQPGGSLRLSCAASGFNFYSYSIHWVRQAPGKGLEWVASISSSSGYTYYADSVKGRFTISADTSKNTAYLQMNSLRAEDTAVYYCARYSWWGWHMEGWWGYMNAFDYWGQGTLVTV

SEQ ID NO: 8 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQAHPKPITFGQGTKVEIKRT

SEQ ID NO: 9 - protein, 255 aa, synthetic construct
EVQLVESGGGLVQPGGSLRLSCAASGFNFYSYSIHWVRQAPGKGLEWVASISSSSGYTYYADSVKGRFTISADTSKNTAYLQMNSLRAEDTAVYYCARYSWWGWHMEGWWGYMNAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLF

SEQ ID NO: 10 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQAHPKPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 7 aa, synthetic construct
NVSYSSI

SEQ ID NO: 12 - protein, 16 aa, synthetic construct
YGWWEYGYGGWWHPAL

SEQ ID NO: 13 - protein, 13 aa, synthetic construct
NYWGWYPFYYFAF

SEQ ID NO: 14 - protein, 6 aa, synthetic construct
VWWGPI

SEQ ID NO: 15 - protein, 125 aa, synthetic construct
EVQLVESGGGLVQPGGSLRLSCAASGFNVYYSSIHWVRQAPGKGLEWVASISSSSGSTSYADSVKGRFTISADTSKNTAYLQMNSLRAEDTAVYYCARYGWWEYGYGGWWHPALDYWGQGTLVTV

SEQ ID NO: 16 - protein, 6 aa, synthetic construct
GWDYPI

SEQ ID NO: 17 - protein, 122 aa, synthetic construct
EVQLVESGGGLVQPGGSLRLSCAASGFNVSYSSIHWVRQAPGKGLEWVASISSSSGYTYYADSVKGRFTISADTSKNTAYLQMNSLRAEDTAVYYCARNYWGWYPFYYFAFDYWGQGTLVTV

SEQ ID NO: 18 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQGWDYPITFGQGTKVEIKRT

SEQ ID NO: 19 - protein, 255 aa, synthetic construct
EVQLVESGGGLVQPGGSLRLSCAASGFNVSYSSIHWVRQAPGKGLEWVASISSSSGYTYYADSVKGRFTISADTSKNTAYLQMNSLRAEDTAVYYCARNYWGWYPFYYFAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPK

SEQ ID NO: 20 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQGWDYPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 6 aa, synthetic construct
VSYSSI

SEQ ID NO: 22 - protein, 10 aa, synthetic construct
RIRGNEGDTY

SEQ ID NO: 23 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQVWWGPITFGQGTKVEIKRT

SEQ ID NO: 24 - protein, 255 aa, synthetic construct
EVQLVESGGGLVQPGGSLRLSCAASGFNVYYSSIHWVRQAPGKGLEWVASISSSSGSTSYADSVKGRFTISADTSKNTAYLQMNSLRAEDTAVYYCARYGWWEYGYGGWWHPALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPP

SEQ ID NO: 25 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQVWWGPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - DNA, 255 bp, synthetic construct
  source 1..255: mol_type = other DNA
gaggttcagctggtggagtctggcggtggcctggtgcagccagggggctcactccgtttgtcctgtgcagcttctggcttcaacttctattcttattctatacactgggtgcgtcaggccccgggtaagggcctggaatgggttgcatctatttcttcttcttctggctatacttattatgccgatagcgtcaagggccgtttcactataagcgcagacacatccaaaaacacagcctacctacaaatgaacagc

SEQ ID NO: 27 - protein, 122 aa, synthetic construct
EVQLVESGGGLVQPGGSLRLSCAASGFNVSYSSIHWVRQAPGKGLEWVARIRGNEGDTYYADSVKGRFTISADTSKNTAYLQMNSLRAEDTAVYYCARNYWGWYPFYYFAFDYWGQGTLVTV

SEQ ID NO: 28 - DNA, 255 bp, synthetic construct
  source 1..255: mol_type = other DNA
gaggttcagctggtggagtctggcggtggcctggtgcagccagggggctcactccgtttgtcctgtgcagcttctggcttcaacgtctcttattcttctatacactgggtgcgtcaggccccgggtaagggcctggaatgggttgcatctatttcttcttcttctggctatacttattatgccgatagcgtcaagggccgtttcactataagcgcagacacatccaaaaacacagcctacctacaaatgaacagc

SEQ ID NO: 29 - protein, 255 aa, synthetic construct
EVQLVESGGGLVQPGGSLRLSCAASGFNVSYSSIHWVRQAPGKGLEWVARIRGNEGDTYYADSVKGRFTISADTSKNTAYLQMNSLRAEDTAVYYCARNYWGWYPFYYFAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPK

SEQ ID NO: 30 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQGWDYPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 255 bp, synthetic construct
  source 1..255: mol_type = other DNA
gaggttcagctggtggagtctggcggtggcctggtgcagccagggggctcactccgtttgtcctgtgcagcttctggcttcaacgtctcttattcttctatacactgggtgcgtcaggccccgggtaagggcctggaatgggttgcacgtattagaggaaacgaaggggacacttactatgccgatagcgtcaagggccgtttcactataagcgcagacacatccaaaaacacagcctacctacaaatgaacagc

SEQ ID NO: 32 - protein, 10 aa, synthetic construct
SIRGAGSDTR

SEQ ID NO: 33 - DNA, 255 bp, synthetic construct
  source 1..255: mol_type = other DNA
gaggttcagctggtggagtctggcggtggcctggtgcagccagggggctcactccgtttgtcctgtgcagcttctggcttcaacgtctcttattcttctatacactgggtgcgtcaggccccgggtaagggcctggaatgggttgcaagtattagaggagccggtagtgacactcgctatgccgatagcgtcaagggccgtttcactataagcgcagacacatccaaaaacacagcctacctacaaatgaacagc

SEQ ID NO: 34 - DNA, 255 bp, synthetic construct
  source 1..255: mol_type = other DNA
gaggttcagctggtggagtctggcggtggcctggtgcagccagggggctcactccgtttgtcctgtgcagcttctggcttcaacgtctcttattcttctatacactgggtgcgtcaggccccgggtaagggcctggaatgggttgcaaaaattcgaggaggccatcaggacactcactatgccgatagcgtcaagggccgtttcactataagcgcagacacatccaaaaacacagcctacctacaaatgaacagc

SEQ ID NO: 35 - DNA, 255 bp, synthetic construct
  source 1..255: mol_type = other DNA
gaggttcagctggtggagtctggcggtggcctggtgcagccagggggctcactccgtttgtcctgtgcagcttctggcttcaacttctcttattcttctatacactgggtgcgtcaggccccgggtaagggcctggaatgggttgcatctatttcttcttcttatggctctacttattatgccgatagcgtcaagggccgtttcactataagcgcagacacatccaaaaacacagcctacctacaaatgaacagc

SEQ ID NO: 36 - DNA, 255 bp, synthetic construct
  source 1..255: mol_type = other DNA
gaggttcagctggtggagtctggcggtggcctggtgcagccagggggctcactccgtttgtcctgtgcagcttctggcttcaacgtctattattcttctatacactgggtgcgtcaggccccgggtaagggcctggaatgggttgcatctatttcttcttcttctggctctacttcttatgccgatagcgtcaagggccgtttcactataagcgcagacacatccaaaaacacagcctacctacaaatgaacagc

SEQ ID NO: 37 - protein, 122 aa, synthetic construct
EVQLVESGGGLVQPGGSLRLSCAASGFNVSYSSIHWVRQAPGKGLEWVASIRGAGSDTRYADSVKGRFTISADTSKNTAYLQMNSLRAEDTAVYYCARNYWGWYPFYYFAFDYWGQGTLVTV

SEQ ID NO: 38 - DNA, 255 bp, synthetic construct
  source 1..255: mol_type = other DNA
tccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgca

SEQ ID NO: 39 - protein, 255 aa, synthetic construct
EVQLVESGGGLVQPGGSLRLSCAASGFNVSYSSIHWVRQAPGKGLEWVASIRGAGSDTRYADSVKGRFTISADTSKNTAYLQMNSLRAEDTAVYYCARNYWGWYPFYYFAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPK

SEQ ID NO: 40 - DNA, 255 bp, synthetic construct; identical to SEQ ID NO: 38
  source 1..255: mol_type = other DNA

SEQ ID NO: 41 - DNA, 255 bp, synthetic construct; identical to SEQ ID NO: 38
  source 1..255: mol_type = other DNA

SEQ ID NO: 42 - protein, 10 aa, synthetic construct
KIRGGHQDTH

SEQ ID NO: 43 - DNA, 255 bp, synthetic construct; identical to SEQ ID NO: 38
  source 1..255: mol_type = other DNA

SEQ ID NO: 44 - DNA, 255 bp, synthetic construct; identical to SEQ ID NO: 38
  source 1..255: mol_type = other DNA

SEQ ID NO: 45 - DNA, 255 bp, synthetic construct; identical to SEQ ID NO: 38
  source 1..255: mol_type = other DNA

SEQ ID NO: 46 - protein, 6 aa, synthetic construct
GWDYPI

SEQ ID NO: 47 - protein, 122 aa, synthetic construct
EVQLVESGGGLVQPGGSLRLSCAASGFNVSYSSIHWVRQAPGKGLEWVAKIRGGHQDTHYADSVKGRFTISADTSKNTAYLQMNSLRAEDTAVYYCARNYWGWYPFYYFAFDYWGQGTLVTV

SEQ ID NO: 48 - DNA, 255 bp, synthetic construct; identical to SEQ ID NO: 38
  source 1..255: mol_type = other DNA

SEQ ID NO: 49 - protein, 255 aa, synthetic construct
EVQLVESGGGLVQPGGSLRLSCAASGFNVSYSSIHWVRQAPGKGLEWVAKIRGGHQDTHYADSVKGRFTISADTSKNTAYLQMNSLRAEDTAVYYCARNYWGWYPFYYFAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPK

SEQ ID NO: 50 - protein, 255 aa, synthetic construct
MLLVNQSHQGFNKEHTSKMVSAIVLYVLLAAAAHSAFAADPHHHHHHGWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKV

SEQ ID NO: 51 - protein, 6 aa, synthetic construct
FSYSSI

SEQ ID NO: 52 - protein, 10 aa, synthetic construct
SISSSYGSTY

SEQ ID NO: 53 - protein, 16 aa, synthetic construct
AFYSHDYRYIYWGSGM

SEQ ID NO: 54 - protein, 255 aa, synthetic construct
MLLVNQSHQGFNKEHTSKMVSAIVLYVLLAAAAHSAFAADPHHHHHHGWELTILHTNDVHSRLEQTSDDSTKCLNASLCVGGVARLFTKVQQIRKEEPNVLFLDAGDQYQGTIWFTVYKGLEVAHFMNILGYDAMALGNHEFDNGVEGLIDPLLRNVKFPILSANIKARGPLAHQISGLFLPSKVLSVGGEVVGIVGYTSKETPFLSNPGTNLVFEDEISALQPEVDKLKTLNVNKIIALGHSGFEMDKLIAQKV

SEQ ID NO: 55 - protein, 255 aa, synthetic construct
MLLVNQSHQGFNKEHTSKMVSAIVLYVLLAAAAHSAFAADPHHHHHHGWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFETDKLIAQKV

SEQ ID NO: 56 - protein, 6 aa, synthetic construct
SWYYPF

SEQ ID NO: 57 - protein, 125 aa, synthetic construct
EVQLVESGGGLVQPGGSLRLSCAASGFNFSYSSIHWVRQAPGKGLEWVASISSSYGSTYYADSVKGRFTISADTSKNTAYLQMNSLRAEDTAVYYCARAFYSHDYRYIYWGSGMDYWGQGTLVTV

SEQ ID NO: 58 - protein, 110 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSWYYPFTFGQGTKVEIKRT

SEQ ID NO: 59 - protein, 255 aa, synthetic construct
EVQLVESGGGLVQPGGSLRLSCAASGFNFSYSSIHWVRQAPGKGLEWVASISSSYGSTYYADSVKGRFTISADTSKNTAYLQMNSLRAEDTAVYYCARAFYSHDYRYIYWGSGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPP

SEQ ID NO: 60 - protein, 215 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSWYYPFTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 6 aa, synthetic construct
VYYSSI

SEQ ID NO: 62 - protein, 10 aa, synthetic construct
SISSSSGSTS

SEQ ID NO: 63 - protein, 255 aa, synthetic construct
MLLVNQSHQGFNKEHTSKMVSAIVLYVLLAAAAHSAFAADPWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVV

SEQ ID NO: 64 - protein, 255 aa, synthetic construct
MLLVNQSHQGFNKEHTSKMVSAIVLYVLLAAAAHSAFAADPHHHHHHELGKTIVYLDGSSQSCRFRECNMGNLICDAMINNNLRHADETFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGR

SEQ ID NO: 65 - protein, 255 aa, synthetic construct; identical to SEQ ID NO: 50